SEQUENCE LISTING (1 sequence)

SEQ ID NO: 1 - protein, 395 aa, Artificial Sequence
  Synthetic polypeptide
MSANATLKPLCPILEQMSRLQSHSNTSIRYIDHAAVLLHGLASLLGLVENGVILFVVGCRMRQTVVTTWVLHLALSDLLASASLPFFTYFLAVGHSWELGTTFCKLHSSIFFLNMFASGFLLSAISLDRCLQVVRPVWAQNHRTVAAAHKVCLVLWALAVLNTVPYFVFRDTISRLDGRIMCYYNVLLLNPGPDRDATCNSRQVALAVSKFLLAFLVPLAIIASSHAAVSLRLQHRGRRRPGRFVRLVAAVVAAFALCWGPYHVFSLLEARAHANPGLRPLVWRGLPFVTSLAFFNSVANPVLYVLTCPDMLRKLRRSLRTVLESVLVDDSELGGAGSSRRRRTSSTARSASPLALCSRPEEPRGPARLLGWLLGSCAASPQTGPLNRALSSTSS

(19) United States
(12) Patent Application Publication
(10) Pub. No.: US 2024/0002376 A1
(43) Pub. Date: Jan. 4, 2024

(54) COMPOSITIONS AND METHODS FOR G-PROTEIN-COUPLED RECEPTOR 44 DETECTION

(71) Applicant: City of Hope, Duarte, CA (US)

(72) Inventors: Fouad Kandeel, Duarte, CA (US); Junfeng Li, Duarte, CA (US); Jiangling Peng, Duarte, CA (US)

(21) Appl. No.: 18/022,708

(22) PCT Filed: Aug. 23, 2021
(86) PCT No.: PCT/US2021/047091

§ 371 Date: Feb. 22, 2023

Related U.S. Application Data
(60) Provisional application No. 63/070,057, filed on Aug. 25, 2020.

(51) Int. Cl. C07D 471/04; A61K 51/04

(52) U.S. Cl. CPC C07D 471/04; A61K 51/0446

(57) ABSTRACT

Disclosed herein, inter alia, are compounds and methods for G-protein-coupled receptor 44 detection.

### CROSS-REFERENCES TO RELATED APPLICATIONS

This application claims the benefit of U.S. Provisional Application No. 63/070,057, filed Aug. 25, 2020, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-774001WO_Sequence_Listing_ST25.TXT, created Aug. 17, 2021, 3,816 bytes, machine format IBM-PC, MS Windows operating system, is hereby incorporated by reference.

### BACKGROUND

Over 100 million U.S. adults live with diabetes or prediabetes. Diabetes is associated with and accelerates many other diseases of the cardiovascular, nervous and renal systems. Reduction in beta cell function and/or numbers (mass) is a hallmark of Type 1 and Type 2 Diabetes (T1D/T2D). Currently, assessments of beta cells are limited to indirect measures such as blood glucose. PET imaging provides safe non-invasive imaging in people. In theory, PET imaging could allow for safe and precise characterization of beta cells. Development of this technology would overcome a major block to advancing diabetic therapies and islet transplantation. Disclosed herein, inter alia, are solutions to these and other problems in the art.

### BRIEF SUMMARY

In an aspect is provided a compound, or a pharmaceutically acceptable salt thereof, having the formula:

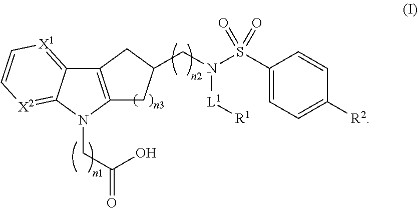

X¹ and X² are each independently CH or N. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is —¹⁸F. The variables n1 and n2 are each independently 0, 1, 2, or 3. The variable n3 is independently 0, 1, or 2.

In an aspect is provided a compound, or a pharmaceutically acceptable salt thereof, having the formula:

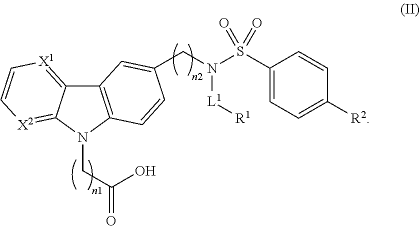

X¹, X², L¹, R¹, R², n1, and n2 are as described herein, including in embodiments.

In an aspect is provided a compound, or a pharmaceutically acceptable salt thereof, having the formula:

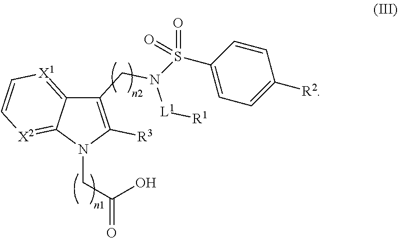

X¹, X², L¹, R¹, R², n1, and n2 are as described herein, including in embodiments. R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In an aspect is provided a compound, or a pharmaceutically acceptable salt thereof, having the formula:

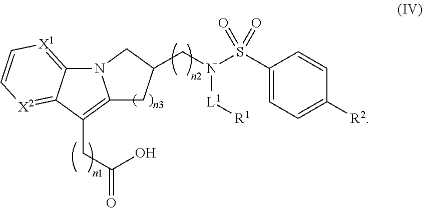

X¹, X², L¹, R¹, R², n1, n2, and n3 are as described herein, including in embodiments.

In an aspect is provided a compound, or a pharmaceutically acceptable salt thereof, having the formula:

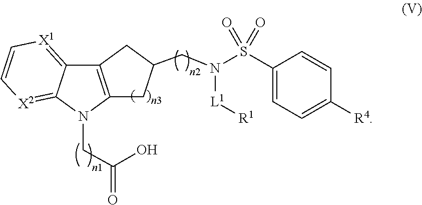

X¹, X², L¹, R¹, n1, n2, and n3 are as described herein, including in embodiments. R⁴ is a leaving group.

In an aspect is provided a compound, or a pharmaceutically acceptable salt thereof, having the formula:

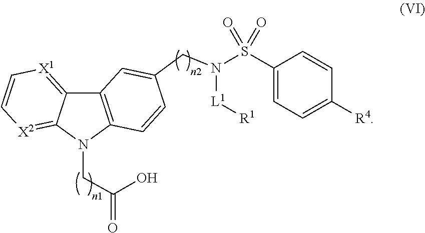

X¹, X², L¹, R¹, R⁴, n1, and n2 are as described herein, including in embodiments.

In an aspect is provided a compound, or a pharmaceutically acceptable salt thereof, having the formula:

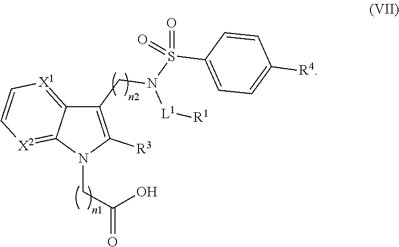

X¹, X², L¹, R¹, R³, R⁴, n1, and n2 are as described herein, including in embodiments.

In an aspect is provided a compound, or a pharmaceutically acceptable salt thereof, having the formula:

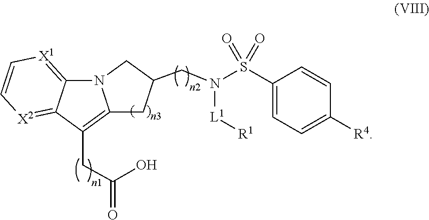

X¹, X², L¹, R¹, R⁴, n1, n2, and n3 are as described herein, including in embodiments.

In an aspect is provided a pharmaceutical composition including a compound described herein, or a pharmaceutically acceptable salt thereof, and a pharmaceutically acceptable excipient.

In an aspect is provided a method of detecting the level of G-protein-coupled receptor 44 in a subject, the method including administering to the subject an effective amount of a compound described herein, or a pharmaceutically acceptable salt thereof. In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a).

In an aspect is provided a method of detecting the level of G-protein-coupled receptor 44 in a cell, tissue, or organ, the method including contacting the cell, tissue, or organ with a compound described herein, or a pharmaceutically acceptable salt thereof. In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a).

In an aspect is provided a method of detecting the level of islets in a subject, the method including administering to the subject an effective amount of a compound described herein, or a pharmaceutically acceptable salt thereof. In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a).

In an aspect is provided a method of detecting the level of beta cells in a subject, the method including administering to the subject an effective amount of a compound described herein, or a pharmaceutically acceptable salt thereof. In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a).

In an aspect is provided a method of detecting the level of beta cell in a subject, the method including the steps:
(i) administering to the subject an effective amount of a compound described herein, or a pharmaceutically acceptable salt thereof; and
(ii) detecting the level of the compound within the pancreas of the subject.

In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a).

In an aspect is provided a method of making a compound, or a pharmaceutically acceptable salt thereof, having the formula:

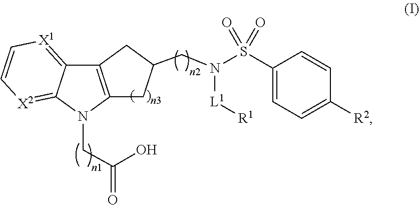

the method including mixing compound (V) and a ¹⁸F fluorinating agent together in a reaction vessel; wherein compound (V) has the formula:

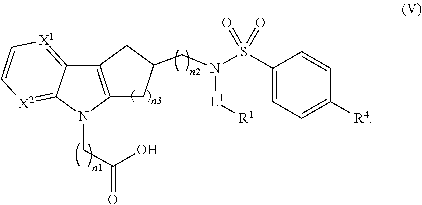

X¹, X², L¹, R¹, R², R⁴, n1, n2, and n3 are as described herein, including in embodiments.

In an aspect is provided a method of making a compound, or a pharmaceutically acceptable salt thereof, having the formula:

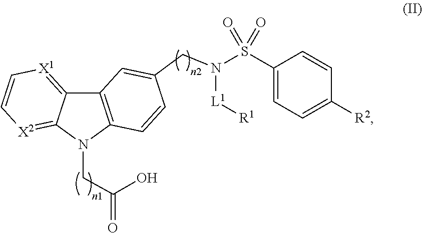

the method including mixing compound (VI) and a ¹⁸F fluorinating agent together in a reaction vessel; wherein compound (VI) has the formula:

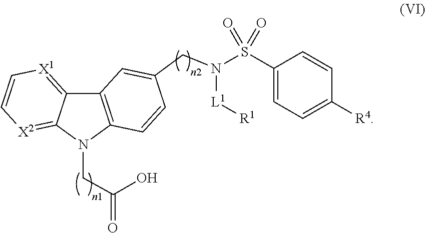

X¹, X², L¹, R¹, R², R⁴, n1, and n2 are as described herein, including in embodiments.

In an aspect is provided a method of making a compound, or a pharmaceutically acceptable salt thereof, having the formula:

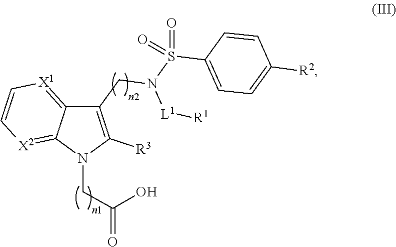

the method including mixing compound (VII) and a ¹⁸F fluorinating agent together in a reaction vessel; wherein compound (VII) has the formula:

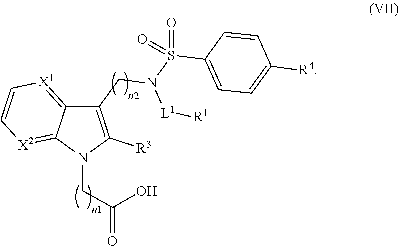

X¹, X², L¹, R¹, R², R³, R⁴, n1, and n2 are as described herein, including in embodiments.

In an aspect is provided a method of making a compound, or a pharmaceutically acceptable salt thereof, having the formula:

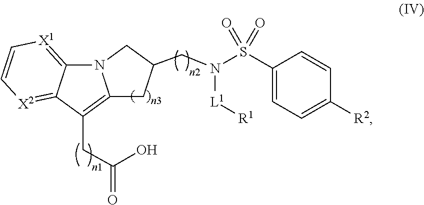

the method including mixing compound (VIII) and a ¹⁸F fluorinating agent together in a reaction vessel; wherein compound (VIII) has the formula:

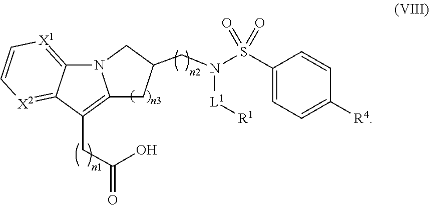

X¹, X², L¹, R¹, R², R⁴, n1, n2, and n3 are as described herein, including in embodiments.

### BRIEF DESCRIPTION OF THE DRAWINGS

FIG. 1. Examples of reported GPR44 antagonists containing a fluorine nuclide. In embodiments, the fluorine nuclide is —¹⁸F.

FIG. 2. GPR44 expression co-localized with beta cells in human islet. Tissue sections of adult human pancreases were immunofluorescence stained for GPR44 antibody, together with islet hormones—insulin, glucagon, and somatostatin. DNA was stained blue with DAPI. Data was verified in tissue section from three to four independent donors.

FIG. 3. GPR44 expression in human 1.1B4 cell line by Western blot analysis.

FIG. 4. Biodistribution of healthy NOD/SCID mice at 30, 60, and 90 minutes post-injection. Cold Ab-1 as blocking agent was treated at 30 minutes post-injection.

FIG. 5. Biodistribution of NOD/SCID mice implanted with 1.1B4 cells 30 minutes after administration of radiolabel (left graph). Tumor indicates implanted human 1.1B4 cells. In other mice, cold Ab-1 as blocking agent was given and organs collected 30 minutes post-injection (right graph).

FIG. 6. Radio-synthesis of [¹⁸F] Ab-1. HPLC conditions: column: Phenomenex Luna 5 μm C18, 100 Å 250×10.0 mm; mobile phase composition: acetonitrile/AMF (0.1 M, pH 4.5)=45/55; flow rate: 4.5 mL/min, retention time: 15.5 min.

FIG. 7. Biodistribution of healthy NOD/SCID mice at 30, 60, and 90 minutes post-injection. Cold Ab-1 as blocking agent was treated at 30 minutes post-injection.

FIG. 8. Biodistribution of NOD/SCID mice implanted with 1.1 B4 cells 30 minutes after administration of radiolabel (left graph). Tumor indicates implanted human 1.1B4 cells. In other mice, cold Ab-1 as blocking agent was given and organs collected 30 minutes post-injection (right graph).

FIG. 9. Biodistribution of NOD/SCID mice implanted with human islets.

FIG. 10. Radio-synthesis of [¹⁸F] Ab-4 and [¹⁸F] Ab-5. HPLC conditions: column: Phenomenex Luna 5 μm C18, 100 Å 250×10.0 mm; mobile phase composition: acetonitrile/AMF (0.1 M, pH 4.5)=45/55; flow rate: 4.5 mL/min.

FIG. 11. Example synthetic schemes.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). In embodiments, the heteroalkyl is fully saturated. In embodiments, the heteroalkyl is monounsaturated. In embodiments, the heteroalkyl is polyunsaturated. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

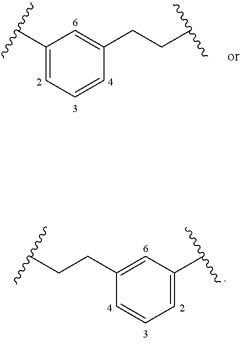 [SMILES: *CCc1cccc(*)c1 ; *CCc1cccc(*)c1]

The term “alkylsulfonyl,” as used herein, means a moiety having the formula —S(O₂)—R′, where R′ is a substituted or unsubstituted alkyl group as defined above. R′ may have a specified number of carbons (e.g., “C1-C4 alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, —N₃, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R′, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), selenium (Se), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₂₀, C₁-C₁₂, C₁-C₈, C₁-C₆, C₁-C₄, or C₁-C₂), heteroalkyl (e.g., 2 to 20 membered, 2 to 12 membered, 2 to 8 membered, 2 to 6 membered, 4 to 6 membered, 2 to 3 membered, or 4 to 5 membered), cycloalkyl (e.g., C₃-C₁₀, C₃-C₈, C₃-C₆, C₄-C₆, or C₅-C₆), heterocycloalkyl (e.g., 3 to 10 membered, 3 to 8 membered, 3 to 6 membered, 4 to 6 membered, 4 to 5 membered, or 5 to 6 membered), aryl (e.g., C₆-C₁₂, C₆-C₁₀, or phenyl), or heteroaryl (e.g., 5 to 12 membered, 5 to 10 membered, 5 to 9 membered, or 5 to 6 membered), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the application (e.g., Examples section, figures, or tables below).

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R¹⁰⁰¹. As a further example, R^{1A} may be substituted with one or more first substituent groups denoted by R^{1A.1}, R^{2A} may be substituted with one or more first substituent groups denoted by R^{2A1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L00.2}, respectively). Thus, each first substituent group, which may alternatively be represented herein as R^{LWW.1} as described above, may be further substituted with one or more second substituent groups, which may alternatively be represented herein as R^{WW.2}.

Finally, each second substituent group (e.g., R¹, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{LWW.1}, which is methyl, the methyl group may be further substituted to form groups including but not limited to:

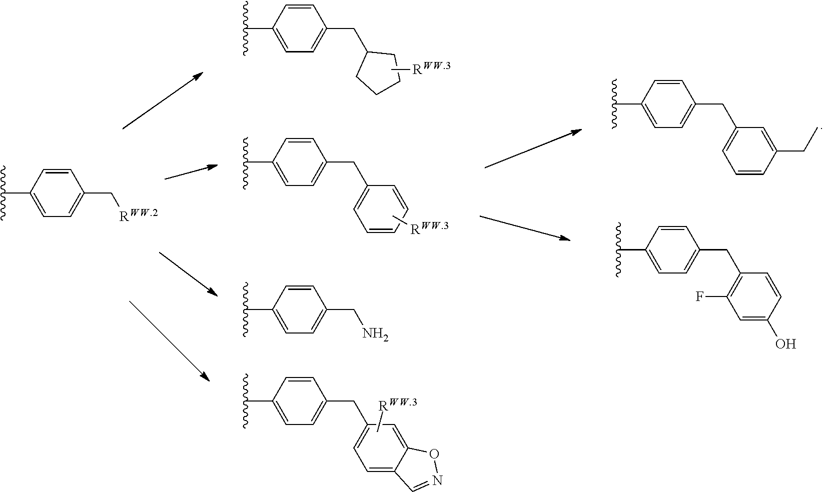

R^{LWW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{LWW.1}; each first substituent group, R^{LWW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{LWW.1}, R^{WW.2} and R^{WW.3} refers to the designated number of one of the two different R^{WW} substituents. For example, in embodiments where R^{100A} and R^{100B} are optionally joined together to form an openly substituted ring, R^{LWW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{LWW.1} is R^{100B.1}, R^{WW.2} is R^{100B.2}, and R^{WW.3} is R^{110B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LW}3-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.32}, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —N₃, R^{LWW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{LWW.1}-substituted or unsubstituted cycloalkyl (e.g., C₃-C₈, C₃-C₆, C₄-C₆, or C₅-C₆), R^{LWW.1}-substituted or unsubstituted heterocycloalkyl (e.g., 3 to 8 membered, 3 to 6 membered, 4 to 6 membered, 4 to 5 membered, or 5 to 6 membered), R^{LWW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{LWW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or strepavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

A “detectable agent” or “detectable moiety” is a substance, element, compound, or composition; or moiety thereof, detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹ Pm, ¹⁵³Sm, ¹⁵⁴⁻¹⁵⁸¹Gd, ¹⁶¹Tb, ¹⁶⁶Dy, ¹⁶⁶Ho ¹⁶⁹Er, ¹⁷⁵Lu ¹⁷⁷Lu, ¹⁸⁶Re, ¹⁸⁸Re, ¹⁸⁹Re, ¹⁹⁴E ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Th, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Th, Dy, Ho, Er, Tm, Yb, and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylates, phenoxides, boronic acid, boronate esters, and alkoxides. In embodiments, the leaving group is designed to facilitate the reaction.

The term “cryptand” is used in accordance with its ordinary meaning in chemistry and refers to a family of synthetic bicyclic and polycyclic multidentate ligands for a variety of cations. Cryptands can form complexes with many cations including, but not limited to, NH₄⁺, lanthanoids, alkali metals, and alkaline earth metals. In embodiments, the cryptand is [2.2.2]-cryptand, wherein [2.2.2]-cryptand is 1,10-diaza-4,7,13,16,21,24-hexaoxabicyclo[8.8.8]hexacosane and has the formula N(CH₂CH₂OCH₂CH₂OCH₂CH₂)₃N.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The terms “polypeptide”, “peptide”, and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments, contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist”, “activator”, “upregulator”, etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The terms “inhibitor”, “repressor”, “antagonist”, or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease means that the disease is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propogated to other signaling pathway components. For example, binding of a thioredoxin protein with a compound as described herein may reduce the interactions between the thioredoxin protein and downstream effectors or signaling pathway components, resulting in changes in cell growth, proliferation, or survival.

In this disclosure, “comprises”, “comprising”, “containing”, and “having” and the like can have the meaning ascribed to them in U.S. patent law and can mean “includes”, “including”, and the like. “Consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with a compound, pharmaceutical composition, or method provided herein. In embodiments, the disease is a metabolic disorder. In some embodiments, the disease is diabetes. In embodiments, the disease is type 1 diabetes (T1D). In embodiments, the disease is type 2 diabetes (T2D). In embodiments, the disease is alopecia. In embodiments, the disease is baldness.

The terms “metabolic disorder” or “metabolic disease” refer to a disorder characterized by one or more abnormal metabolic processes in a subject. In embodiments, a metabolic disorder may be associated with, related to, or may be diabetes (e.g., type 1 diabetes or type 2 diabetes), insulin resistance, metabolic syndrome, obesity, hyperlipidemia, hyperglycemia, high serum triglycerides, and/or high blood pressure. In embodiments, the metabolic disorder is diabetes. In embodiments, the metabolic disorder is type 1 diabetes (TID). In embodiments, the metabolic disorder is type 2 diabetes (T2D).

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. In embodiments, treating refers to treating a subject having a disease.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

“Patient” or “subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” is meant that a composition described herein is administered at the same time, just prior to, or just after the administration of one or more additional therapies. The compounds provided herein can be administered alone or can be coadministered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “islet” is used in accordance with its plain ordinary meaning and refers to a cluster of cells usually found in the pancreas and includes different types of cells that work together to regulate blood sugar. One cell type is a beta cell.

The term “beta cell” is used in accordance with its plain ordinary meaning and refers to a cell found in islets that synthesize and secrete insulin and amylin.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

The terms “G-protein-coupled receptor 44”, “GPR44”, “prostaglandin D₂ receptor 2”, “DP₂”, and “CRTH2” refer to a protein belonging to the class of prostaglandin receptors. In embodiments, GPR44 is found in human beta cells. In embodiments, GPR44 is found in human beta cells in the pancreas. The term includes any recombinant or naturally-occurring form of GPR44 or variants thereof that maintain GPR44 activity (e.g., within at least 30%, 40%, 50%, 60%, 70%, 80%, 90%, 95%, or 100% activity compared to wildtype GPR44). In embodiments, the GPR44 protein has the amino acid sequence set forth in or corresponding to Entrez 11251, UniProt Q9Y5Y4, RefSeq (protein) NP_004769.2. In embodiments, the GPR44 gene has the nucleic acid sequence set forth in RefSeq (mRNA) NM_004778.2. In embodiments, the amino acid sequence or nucleic acid sequence is the sequence known at the time of filing of the present application. In embodiments, the GPR44 protein corresponds to the sequence:

(SEQ ID NO: 1)
MSANATLKPLCPILEQMSRLQSHSNTSIRYIDHAAVLLHGLASLLGLV
ENGVILFVVGCRMRQTVVTTWVLHLALSDLLASASLPFFTYFLAVGHS
WELGTTFCKLHSSIFFLNMFASGFLLSAISLDRCLQVVRPVWAQNHRT
VAAAHKVCLVLWALAVLNTVPYFVFRDTISRLDGRIMCYYNVLLLNPG
PDRDATCNSRQVALAVSKFLLAFLVPLAIIASSHAAVSLRLQHRGRRR
PGRFVRLVAAVVAAFALCWGPYHVFSLLEARAHANPGLRPLVWRGLPF
VTSLAFFNSVANPVLYVLTCPDMLRKLRRSLRTVLESVLVDDSELGGA
GSSRRRRTSSTARSASPLALCSRPEEPRGPARLLGWLLGSCAASPQTG
PLNRALSSTSS.

The term “positron emission tomography” or “PET” is used in accordance with its plain ordinary meaning and refers to an imaging technique that uses radioactive substances to visualize and measure metabolic processes.

### II. Compounds

In an aspect is provided a compound, or a pharmaceutically acceptable salt thereof, having the formula:

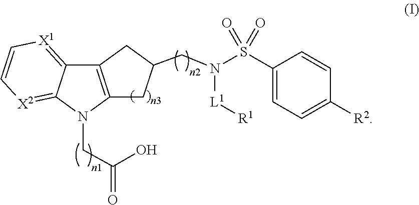

X¹ and X² are each independently CH or N.

L¹ is a bond, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, or 2 to 3 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R² is —¹⁸F.

The variables n1 and n2 are each independently 0, 1, 2, or 3.

The variable n3 is independently 0, 1, or 2.

In embodiments, the compound has the formula:

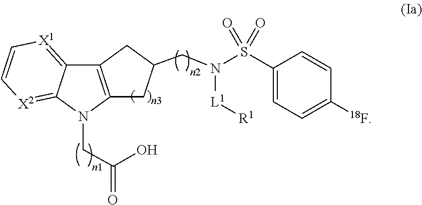

X¹, X², L¹, R¹, n1, n2, and n3 are as described herein, including in embodiments.

In embodiments, the compound has the formula:

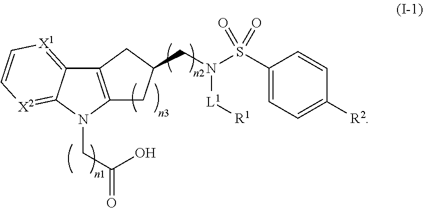

X¹, X², L¹, R¹, R², n1, n2, and n3 are as described herein, including in embodiments.

In embodiments, the compound has the formula:

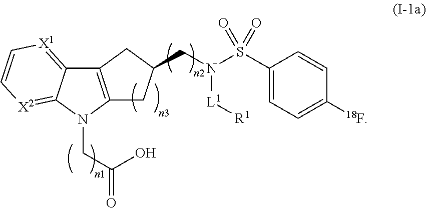

X¹, X², L¹, R¹, n1, n2, and n3 are as described herein, including in embodiments.

In embodiments, the compound has the formula:

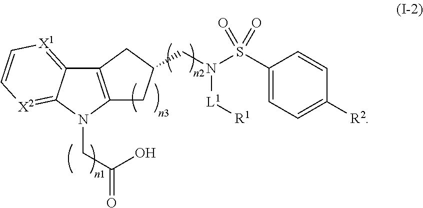

X¹, X², L¹, R¹, R², n1, n2, and n3 are as described herein, including in embodiments.

In embodiments, the compound has the formula:

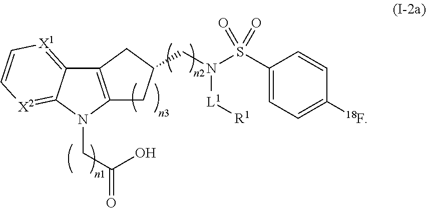

X¹, X², L¹, R¹, n1, n2, and n3 are as described herein, including in embodiments.

In an aspect is provided a compound, or a pharmaceutically acceptable salt thereof, having the formula:

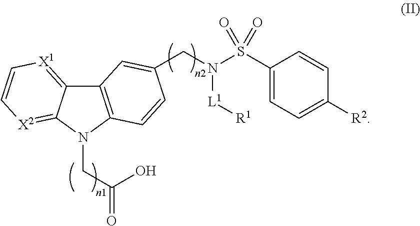

X¹, X², L¹, R¹, R², n1, and n2 are as described herein, including in embodiments.

In an aspect is provided a compound, or a pharmaceutically acceptable salt thereof, having the formula:

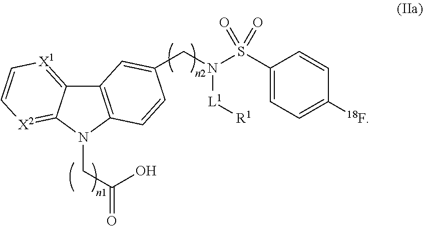

X¹, X², L¹, R¹, n1, and n2 are as described herein, including in embodiments.

In an aspect is provided a compound, or a pharmaceutically acceptable salt thereof, having the formula:

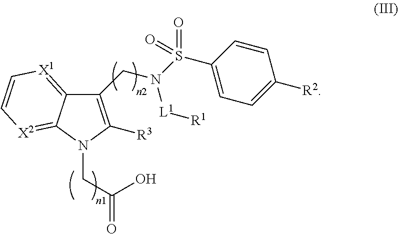

X¹, X², L¹, R¹, R², n1, and n2 are as described herein, including in embodiments.

In an aspect is provided a compound, or a pharmaceutically acceptable salt thereof, having the formula:

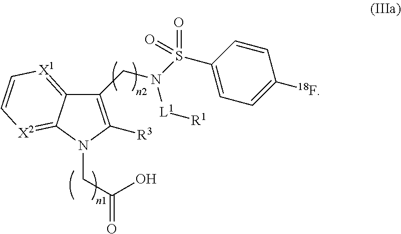

X¹, X², L¹, R¹, n1, and n2 are as described herein, including in embodiments.

R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In an aspect is provided a compound, or a pharmaceutically acceptable salt thereof, having the formula:

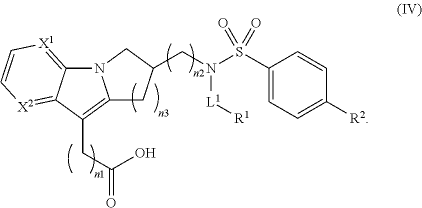

X¹, X², L¹, R¹, R², n1, n2, and n3 are as described herein, including in embodiments.

In an aspect is provided a compound, or a pharmaceutically acceptable salt thereof, having the formula:

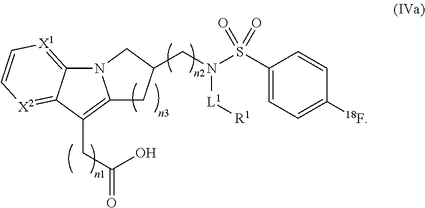

X¹, X², L¹, R¹, n1, n2, and n3 are as described herein, including in embodiments.

In embodiments, the compound has the formula:

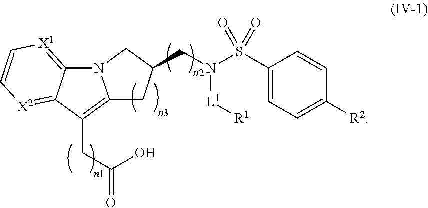

X¹, X², L¹, R¹, R², n1, n2, and n3 are as described herein, including in embodiments.

In embodiments, the compound has the formula:

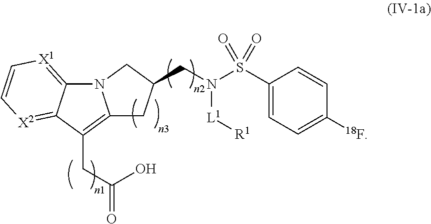

X¹, X², L¹, R¹, R², n1, n2, and n3 are as described herein, including in embodiments.

In embodiments, the compound has the formula:

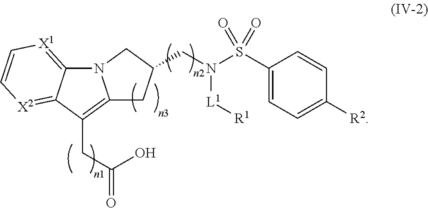

X¹, X², L¹, R¹, R², n1, n2, and n3 are as described herein, including in embodiments.

In embodiments, the compound has the formula:

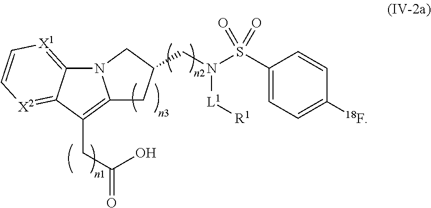

X¹, X², L¹, R¹, n1, n2, and n3 are as described herein, including in embodiments.

In an aspect is provided a compound, or a pharmaceutically acceptable salt thereof, having the formula:

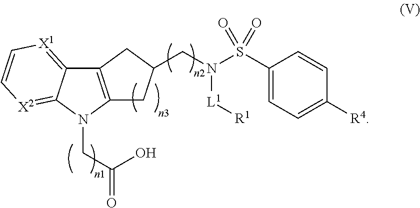

X¹, X², L¹, R¹, n1, n2, and n3 are as described herein, including in embodiments.

R⁴ is a leaving group.

In embodiments, the compound has the formula:

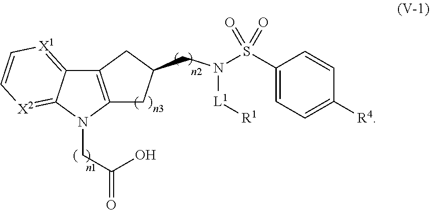

X¹, X², L¹, R¹, R⁴, n1, n2, and n3 are as described herein, including in embodiments.

In embodiments, the compound has the formula:

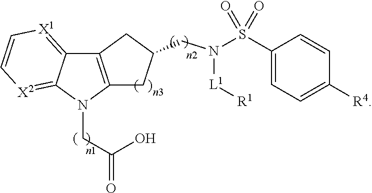

X¹, X², L¹, R¹, R⁴, n1, n2, and n3 are as described herein, including in embodiments.

In an aspect is provided a compound, or a pharmaceutically acceptable salt thereof, having the formula:

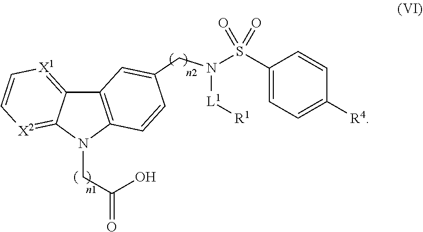

X¹, X², L¹, R¹, R⁴, n1, and n2 are as described herein, including in embodiments.

In an aspect is provided a compound, or a pharmaceutically acceptable salt thereof, having the formula:

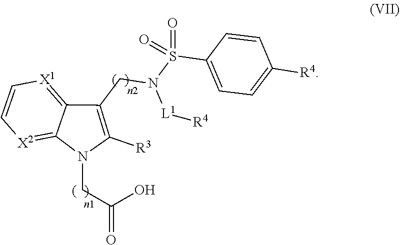

X¹, X², L¹, R¹, R³, R⁴, n1, and n2 are as described herein, including in embodiments.

In an aspect is provided a compound, or a pharmaceutically acceptable salt thereof, having the formula:

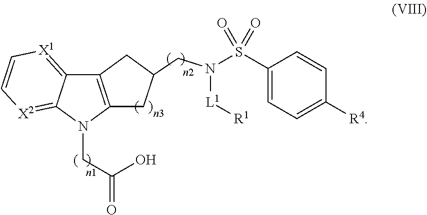

X¹, X², L¹, R¹, R⁴, n1, n2, and n3 are as described herein, including in embodiments.

In embodiments, the compound has the formula:

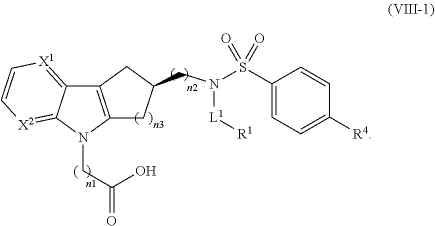

X¹, X², L¹, R¹, R⁴, n1, n2, and n3 are as described herein, including in embodiments.

In embodiments, the compound has the formula:

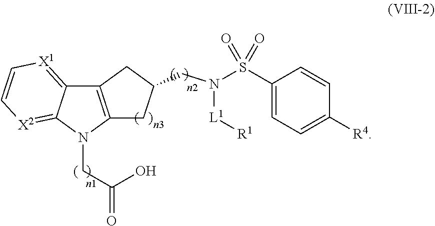

X¹, X², L¹, R¹, R⁴, n1, n2, and n3 are as described herein, including in embodiments.

In embodiments, X¹ is CH. In embodiments, X¹ is N.

In embodiments, X² is CH. In embodiments, X² is N.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is a bond or unsubstituted C₁-C₄ alkylene. In embodiments, L¹ is a bond. In embodiments, L¹ is unsubstituted C₁-C₄ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene. In embodiments, L¹ is unsubstituted butylene. In embodiments, L¹ is unsubstituted n-butylene. In embodiments, L¹ is unsubstituted tert-butylene.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R¹ is hydrogen, substituted or unsubstituted alkyl, or substituted or unsubstituted aryl. In embodiments, R¹ is hydrogen, substituted or unsubstituted C₁-C₄ alkyl, or substituted or unsubstituted phenyl. In embodiments, R¹ is hydrogen. In embodiments, R¹ is substituted or unsubstituted C₁-C₄ alkyl. In embodiments, R¹ is unsubstituted C₁-C₄ alkyl. In embodiments, R¹ is unsubstituted methyl. In embodiments, R¹ is unsubstituted ethyl. In embodiments, R¹ is unsubstituted propyl. In embodiments, R¹ is unsubstituted n-propyl. In embodiments, R¹ is unsubstituted isopropyl. In embodiments, R¹ is unsubstituted butyl. In embodiments, R¹ is unsubstituted n-butyl. In embodiments, R¹ is unsubstituted tert-butyl. In embodiments, R¹ is substituted or unsubstituted phenyl. In embodiments, R¹ is unsubstituted phenyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen or unsubstituted C₁-C₄ alkyl. In embodiments, R³ is hydrogen or unsubstituted methyl. In embodiments, R³ is hydrogen. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted tert-butyl. In embodiments, R³ is unsubstituted C₁-C₄ alkoxy. In embodiments, R³ is unsubstituted methoxy. In embodiments, R³ is unsubstituted ethoxy. In embodiments, R³ is unsubstituted propoxy. In embodiments, R³ is unsubstituted n-propoxy. In embodiments, R³ is unsubstituted isopropoxy. In embodiments, R³ is unsubstituted butoxy. In embodiments, R³ is unsubstituted n-butoxy. In embodiments, R³ is unsubstituted tert-butoxy.

In embodiments, R⁴ is —NO₂, —Cl, —Br, —I,

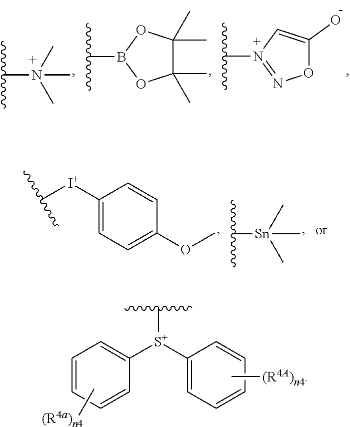

In embodiments, R⁴ is —NO₂. In embodiments, R⁴ is —Cl. In embodiments, R⁴ is —Br. In embodiments, R⁴ is —I. In embodiments, R⁴ is

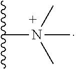

In embodiments, R⁴ is

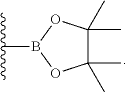 [SMILES: *B1OC(C)(C)C(C)(C)O1]

In embodiments, R⁴ is

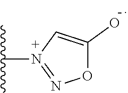

In embodiments, R⁴ is

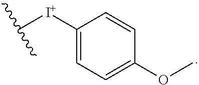 [SMILES: *[I+]c1ccc(OC)cc1]

In embodiments, R⁴ is

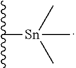 [SMILES: [*][Sn]([CH3])([CH3])[CH3]]

In embodiments, R⁴ is

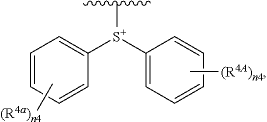

wherein R^{4A} and n4 are as described herein, including in embodiments, and wherein each R^{4A} may optionally be different. In embodiments, R⁴ is

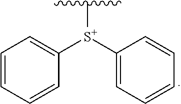 [SMILES: *[S+](c1ccccc1)c1ccccc1]

In embodiments, R⁴ is

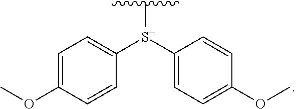

In embodiments, R⁴ is

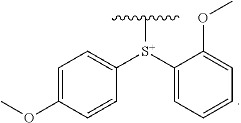

In embodiments, R⁴ is a leaving group as described in Sander, K., Gendron, T., Yiannaki, E. et al., Sci. Rep., 5, 9941 (2015), which is herein incorporated by reference in its entirety for all purposes.

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The variable n4 is independently an integer from 0 to 5.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, R^{4A} is independently unsubstituted C₁-C₄ alkyl. In embodiments, R^{4A} is independently unsubstituted methyl. In embodiments, R^{4A} is independently unsubstituted ethyl. In embodiments, R^{4A} is independently unsubstituted propyl. In embodiments, R^{4A} is independently unsubstituted n-propyl. In embodiments, R^{4A} is independently unsubstituted isopropyl. In embodiments, R^{4A} is independently unsubstituted butyl. In embodiments, R^{4A} is independently unsubstituted n-butyl. In embodiments, R^{4A} is independently unsubstituted tert-butyl. In embodiments, R^{4A} is independently unsubstituted C₁-C₄ alkoxy. In embodiments, R^{4A} is independently unsubstituted methoxy. In embodiments, R^{4A} is independently unsubstituted ethoxy. In embodiments, R^{4A} is independently unsubstituted propoxy. In embodiments, R^{4A} is independently unsubstituted n-propoxy. In embodiments, R^{4A} is independently unsubstituted isopropoxy. In embodiments, R^{4A} is independently unsubstituted butoxy. In embodiments, R^{4A} is independently unsubstituted n-butoxy. In embodiments, R^{4A} is independently unsubstituted tert-butoxy.

In embodiments, where R⁴ is a charged moiety, a counterion may be present. In embodiments, when R⁴ is

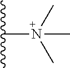

a negative counterion is present. In embodiments, when R⁴ is

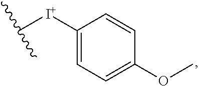 [SMILES: *[I+]c1ccc(OC)cc1]

a negative counterion is present. In embodiments, when R⁴ is

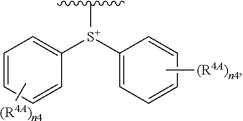

a negative counterion is present. In embodiments, the negative counterion is F₃CS(O)₂O⁻ (TfO⁻ or triflate ion). In embodiments, the negative counterion is I⁻ (iodide ion). In embodiments, the negative counterion is Br⁻ (bromide ion). In embodiments, the negative counterion is Cl⁻ (chloride ion). In embodiments, the negative counterion is HSO₄⁻ (hydrogen sulfate ion). In embodiments, the negative counterion is HO⁻ (hydroxide ion). In embodiments, the negative counterion is R^{4B}C(O)O (carboxylate ion), wherein R^{4B} is as described herein, including in embodiments. In embodiments, the negative counterion is HC(O)O⁻ (formate ion). In embodiments, the negative counterion is CH₃C(O)O⁻ (acetate ion). In embodiments, the negative counterion is CH₃CH(OH)C(O)O-(lactate ion). In embodiments, the negative counterion is C₂O₄²⁻ (oxalate ion). In embodiments, the negative counterion is C₃H50(C(O)O)₃₃— (citrate ion).

R^{4B} is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, or 2 to 3 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, n1 is 0. In embodiments, n1 is 1. In embodiments, n1 is 2. In embodiments, n1 is 3.

In embodiments, n2 is 0. In embodiments, n2 is 1. In embodiments, n2 is 2. In embodiments, n2 is 3.

In embodiments, n3 is 0. In embodiments, n3 is 1. In embodiments, n3 is 2.

In embodiments, n4 is independently 0. In embodiments, n4 is independently 1. In embodiments, n4 is independently 2. In embodiments, n4 is independently 3. In embodiments, n4 is independently 4. In embodiments, n4 is independently 5.

In embodiments, the compound is

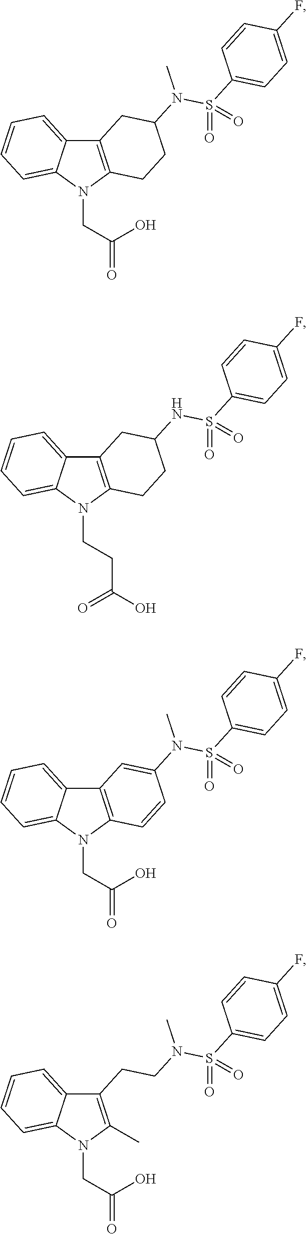 [SMILES: CN(C1CCc2c(c3ccccc3n2CC(=O)O)C1)S(=O)(=O)c1ccc(F)cc1 ; O=C(O)CCn1c2c(c3ccccc31)CC(NS(=O)(=O)c1ccc(F)cc1)CC2 ; CN(c1ccc2c(c1)c1ccccc1n2CC(=O)O)S(=O)(=O)c1ccc(F)cc1 ; Cc1c(CCN(C)S(=O)(=O)c2ccc(F)cc2)c2ccccc2n1CC(=O)O]

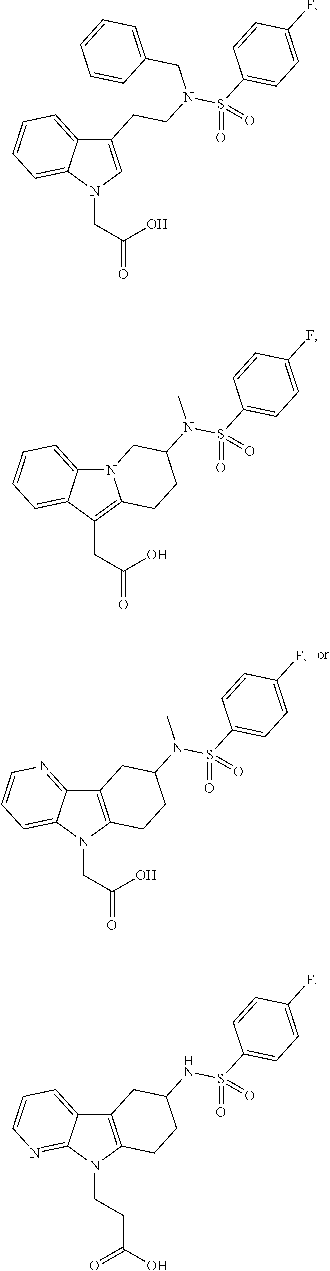 [SMILES: O=C(O)Cn1cc(CCN(Cc2ccccc2)S(=O)(=O)c2ccc(F)cc2)c2ccccc21 ; CN(C1CCc2c(CC(=O)O)c3ccccc3n2C1)S(=O)(=O)c1ccc(F)cc1 ; CN(C1CCc2c(c3ncccc3n2CC(=O)O)C1)S(=O)(=O)c1ccc(F)cc1 ; "or" ; O=C(O)CCn1c2c(c3cccnc31)CC(NS(=O)(=O)c1ccc(F)cc1)CC2]

In embodiments, the compound is

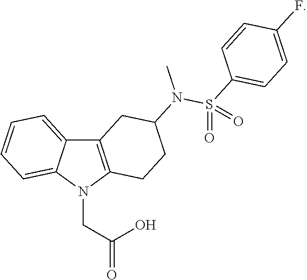 [SMILES: CN(C1CCc2c(c3ccccc3n2CC(=O)O)C1)S(=O)(=O)c1ccc(F)cc1]

In embodiments, the compound is

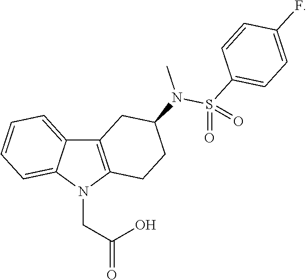 [SMILES: CN([C@H]1CCc2c(c3ccccc3n2CC(=O)O)C1)S(=O)(=O)c1ccc(F)cc1]

In embodiments, the compound is

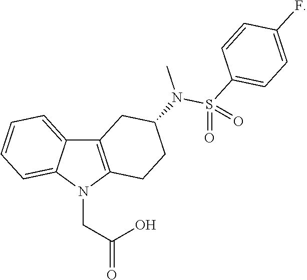 [SMILES: CN([C@@H]1CCc2c(c3ccccc3n2CC(=O)O)C1)S(=O)(=O)c1ccc(F)cc1]

In embodiments, the compound is

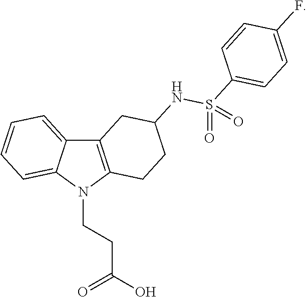 [SMILES: O=C(O)CCn1c2c(c3ccccc31)CC(NS(=O)(=O)c1ccc(F)cc1)CC2]

In embodiments, the compound is

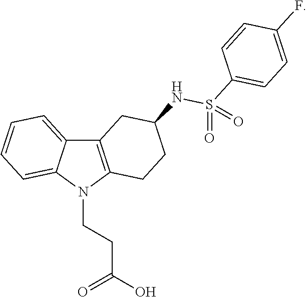 [SMILES: O=C(O)CCn1c2c(c3ccccc31)C[C@@H](NS(=O)(=O)c1ccc(F)cc1)CC2]

In embodiments, the compound is

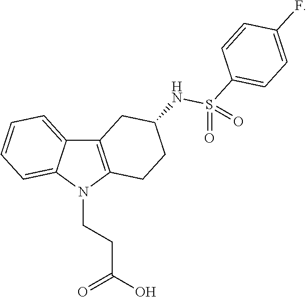 [SMILES: O=C(O)CCn1c2c(c3ccccc31)C[C@H](NS(=O)(=O)c1ccc(F)cc1)CC2]

In embodiments, the compound is

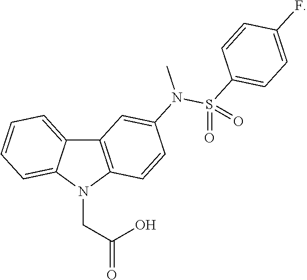 [SMILES: CN(c1ccc2c(c1)c1ccccc1n2CC(=O)O)S(=O)(=O)c1ccc(F)cc1]

In embodiments, the compound is

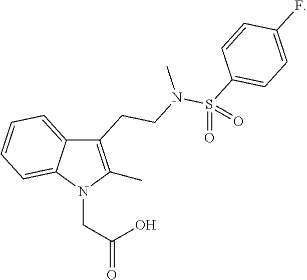 [SMILES: Cc1c(CCN(C)S(=O)(=O)c2ccc(F)cc2)c2ccccc2n1CC(=O)O]

In embodiments, the compound is

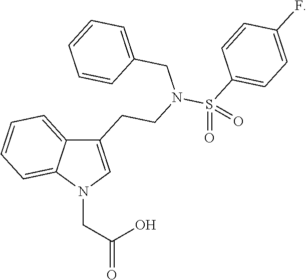 [SMILES: O=C(O)Cn1cc(CCN(Cc2ccccc2)S(=O)(=O)c2ccc(F)cc2)c2ccccc21]

In embodiments, the compound is

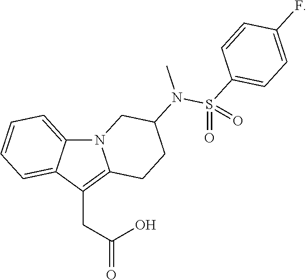 [SMILES: CN(C1CCc2c(CC(=O)O)c3ccccc3n2C1)S(=O)(=O)c1ccc(F)cc1]

In embodiments, the compound is

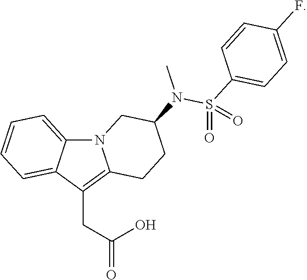 [SMILES: CN([C@H]1CCc2c(CC(=O)O)c3ccccc3n2C1)S(=O)(=O)c1ccc(F)cc1]

In embodiments, the compound is

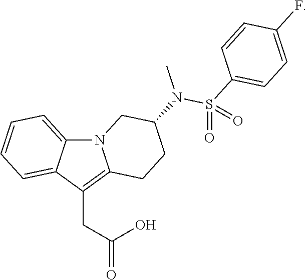 [SMILES: CN([C@@H]1CCc2c(CC(=O)O)c3ccccc3n2C1)S(=O)(=O)c1ccc(F)cc1]

In embodiments, the compound is

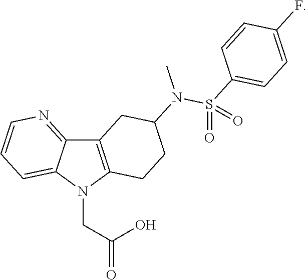 [SMILES: CN(C1CCc2c(c3ncccc3n2CC(=O)O)C1)S(=O)(=O)c1ccc(F)cc1]

In embodiments, the compound is

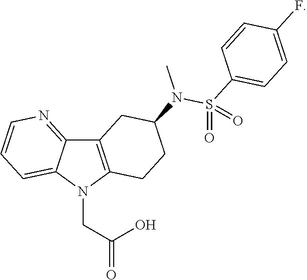 [SMILES: CN([C@H]1CCc2c(c3ncccc3n2CC(=O)O)C1)S(=O)(=O)c1ccc(F)cc1]

In embodiments, the compound is

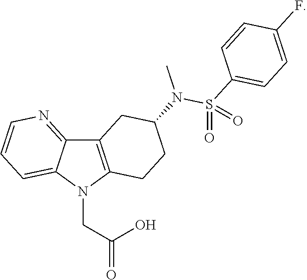 [SMILES: CN([C@@H]1CCc2c(c3ncccc3n2CC(=O)O)C1)S(=O)(=O)c1ccc(F)cc1]

In embodiments, the compound is

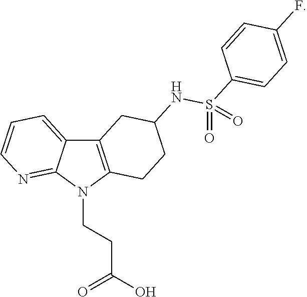 [SMILES: O=C(O)CCn1c2c(c3cccnc31)CC(NS(=O)(=O)c1ccc(F)cc1)CC2]

In embodiments, the compound is

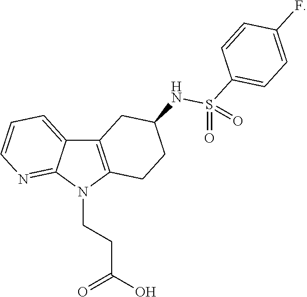 [SMILES: O=C(O)CCn1c2c(c3cccnc31)C[C@@H](NS(=O)(=O)c1ccc(F)cc1)CC2]

In embodiments, the compound is

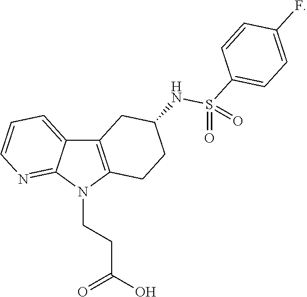 [SMILES: O=C(O)CCn1c2c(c3cccnc31)C[C@H](NS(=O)(=O)c1ccc(F)cc1)CC2]

In embodiments, the compound is

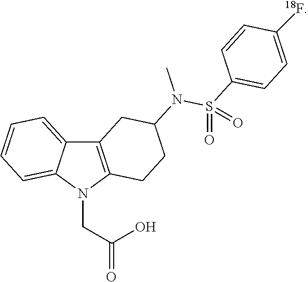

In embodiments, the compound is

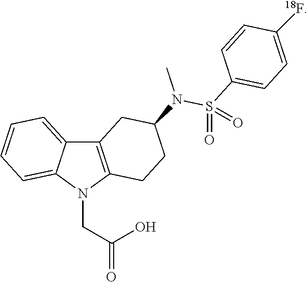

In embodiments, the compound is

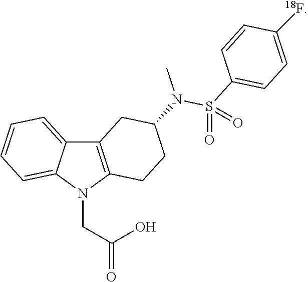

In embodiments, the compound is

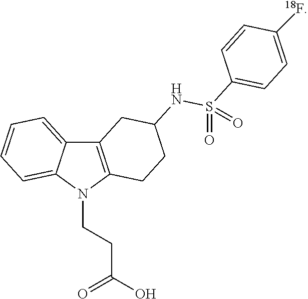

In embodiments, the compound is

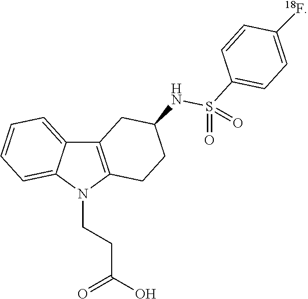

In embodiments, the compound is

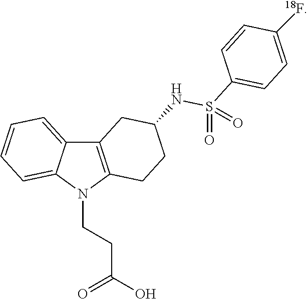

In embodiments, the compound is

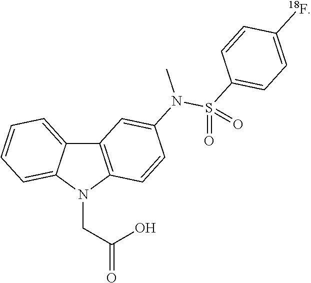

In embodiments, the compound is

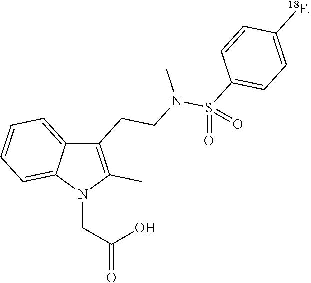

In embodiments, the compound is

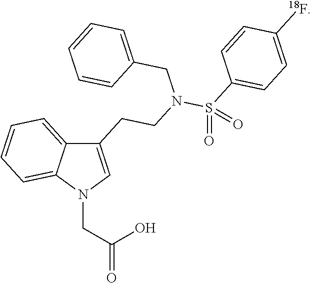

In embodiments, the compound is

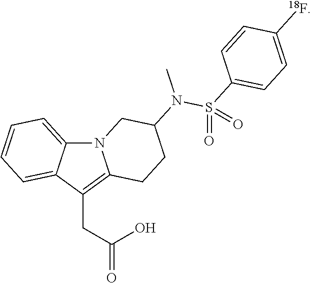

In embodiments, the compound is

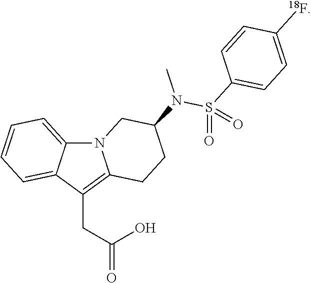

In embodiments, the compound is

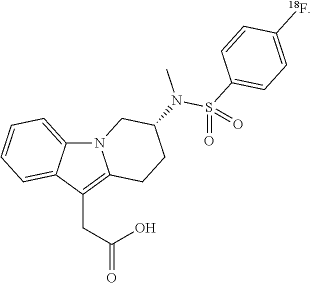

In embodiments, the compound is

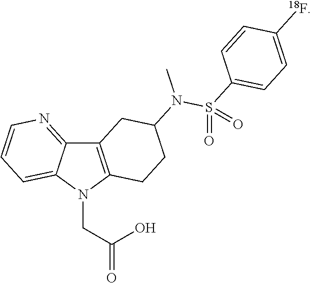

In embodiments, the compound is

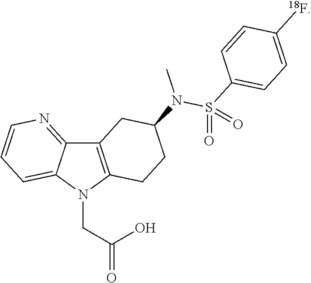

In embodiments, the compound is

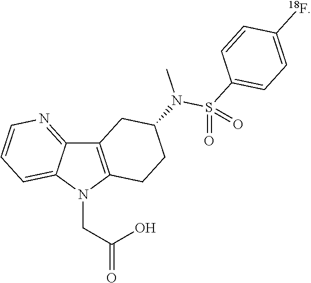

In embodiments, the compound is

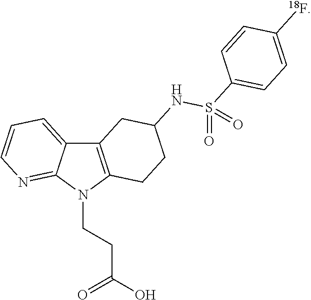

In embodiments, the compound is

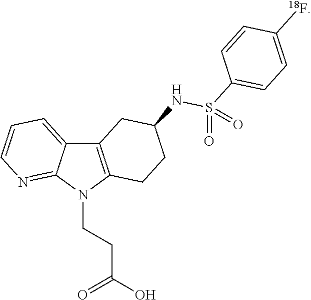

In embodiments, the compound is

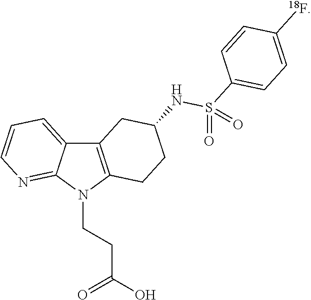

In embodiments, the compound is

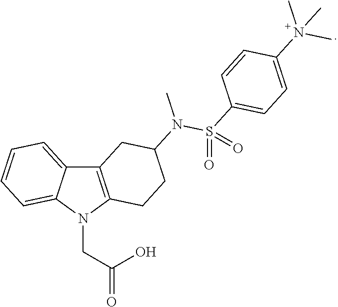

In embodiments, the compound is

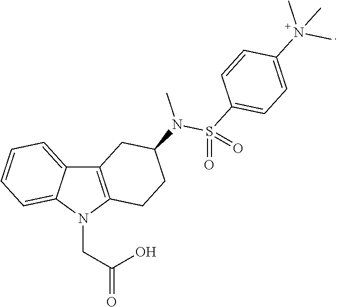

In embodiments, the compound is

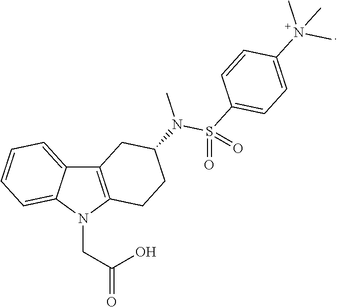

In embodiments, the compound is

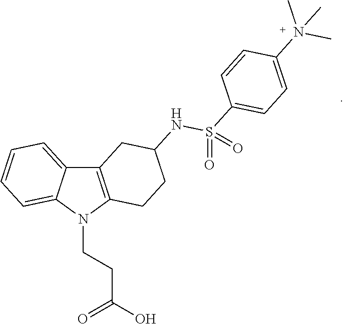

In embodiments, the compound is

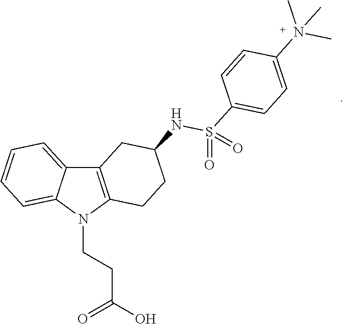

In embodiments, the compound is

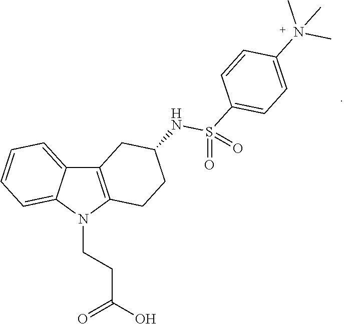

In embodiments, the compound is

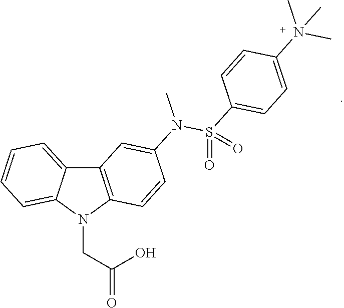

In embodiments, the compound is

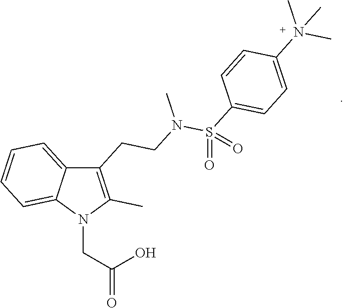

In embodiments, the compound is

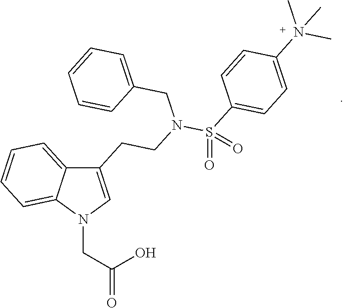

In embodiments, the compound is

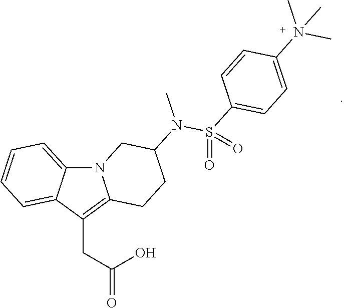

In embodiments, the compound is

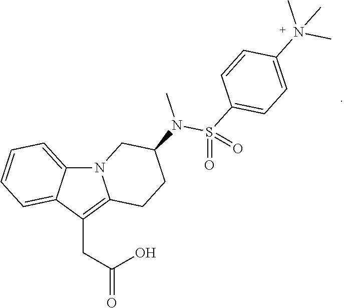

In embodiments, the compound is

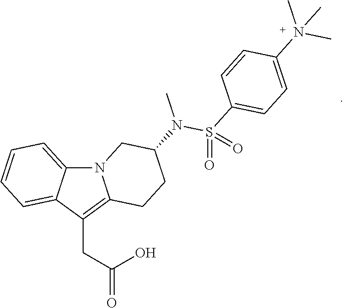

In embodiments, the compound is

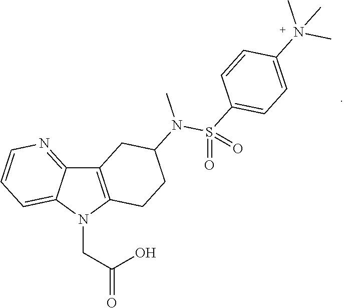

In embodiments, the compound is

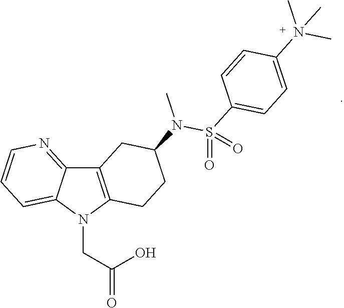

In embodiments, the compound is

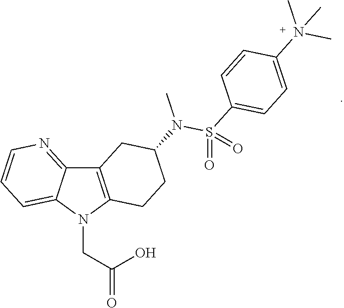

In embodiments, the compound is

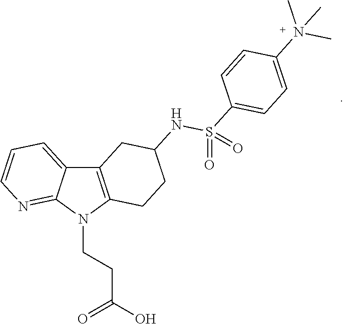

In embodiments, the compound is

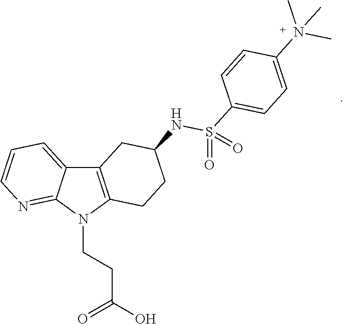

In embodiments, the compound is

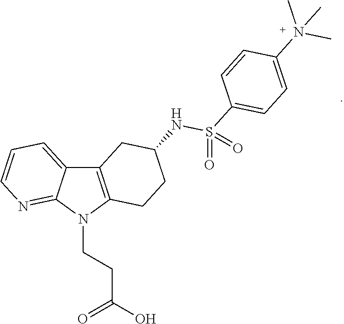

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R¹³ have values corresponding to the values of R″, R^{LWW.1}, R^{WW.2}, and R^{WW.3}, respectively, as explained in the definitions section above in the description of “first substituent group(s)”, wherein R^{WW}, R^{LWW.1}, R^{WW.2}, and R^{WW.3} correspond to R¹, R^{1.1}, R^{1.2}, and R^{1.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{LWW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{LWW.1}, R^{WW.2}, and R^{WW.3}, respectively, as explained in the definitions section above in the description of “first substituent group(s)”, wherein RWW, R^{LWW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B}0.2, and R^{4B.3} have values corresponding to the values of R″, R^{LWW.1}, R^{WW.2}, and R^{WW.3}, respectively, as explained in the definitions section above in the description of “first substituent group(s)”, wherein R^{WW}, R^{LWW.1}, R^{WW.2}, and R^{WW.3} correspond to R^{4B}, R^{4B.1}, R^{4B.2}, and R^{4B.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, the compound is useful as a detectable agent. In embodiments, the compound is useful as a positron emission tomography (PET) agent. In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a). In embodiments, the compound is a compound of formula (I). In embodiments, the compound is a compound of formula (La). In embodiments, the compound is a compound of formula (I-1). In embodiments, the compound is a compound of formula (I-1a). In embodiments, the compound is a compound of formula (I-2). In embodiments, the compound is a compound of formula (I-2a). In embodiments, the compound is a compound of formula (II). In embodiments, the compound is a compound of formula (IIa). In embodiments, the compound is a compound of formula (III). In embodiments, the compound is a compound of formula (IIIa). In embodiments, the compound is a compound of formula (IV). In embodiments, the compound is a compound of formula (IVa). In embodiments, the compound is a compound of formula (IV-1). In embodiments, the compound is a compound of formula (IV-1a). In embodiments, the compound is a compound of formula (IV-2). In embodiments, the compound is a compound of formula (IV-2a).

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound described herein (e.g., in an aspect, embodiment, example, table, figure, or claim).

### III. Pharmaceutical Compositions

In an aspect is provided a pharmaceutical composition including a compound described herein, or a pharmaceutically acceptable salt thereof, and a pharmaceutically acceptable excipient.

In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a). In embodiments, the compound is a compound of formula (I). In embodiments, the compound is a compound of formula (Ia). In embodiments, the compound is a compound of formula (I-1). In embodiments, the compound is a compound of formula (I-1a). In embodiments, the compound is a compound of formula (I-2). In embodiments, the compound is a compound of formula (I-2a). In embodiments, the compound is a compound of formula (II). In embodiments, the compound is a compound of formula (IIa). In embodiments, the compound is a compound of formula (III). In embodiments, the compound is a compound of formula (IIIa). In embodiments, the compound is a compound of formula (IV). In embodiments, the compound is a compound of formula (IVa). In embodiments, the compound is a compound of formula (IV-1). In embodiments, the compound is a compound of formula (IV-1a). In embodiments, the compound is a compound of formula (IV-2). In embodiments, the compound is a compound of formula (IV-2a).

In embodiments, the compound, or pharmaceutically acceptable salt thereof, is included in a therapeutically effective amount.

In embodiments, the pharmaceutical composition includes a second agent (e.g., therapeutic agent). In embodiments, the pharmaceutical composition includes a second agent (e.g., therapeutic agent) in a therapeutically effective amount. In embodiments, the second agent is an agent for treating a metabolic disorder. In embodiments, the second agent is an agent for treating diabetes. In embodiments, the second agent is an agent for treating type 1 diabetes. In embodiments, the second agent is an agent for treating type 2 diabetes. In embodiments, the second agent is an agent for treating alopecia. In embodiments, the second agent is an agent for treating baldness.

### IV. Methods of Use

In an aspect is provided a method of detecting the level of G-protein-coupled receptor 44 in a subject, the method including administering to the subject an effective amount of a compound described herein, or a pharmaceutically acceptable salt thereof. In embodiments, the method includes administering to the subject a therapeutically effective amount of a compound described herein, or a pharmaceutically acceptable salt thereof.

In embodiments, the method further includes detecting a signal emitted by ¹⁸F in the compound. In embodiments, the method further includes generating an image representative of the location and/or amount of the compound based on the signal. In embodiments, the method further includes determining the distribution and/or extent of a disease in the subject.

In embodiments, the disease is a metabolic disorder. In embodiments, the metabolic disorder is type 1 diabetes. In embodiments, the metabolic disorder is type 2 diabetes.

In an aspect is provided a method of detecting the level of G-protein-coupled receptor 44 in a cell, tissue, or organ, the method including contacting the cell, tissue, or organ with a compound described herein, or a pharmaceutically acceptable salt thereof. In embodiments, the cell is a beta cell. In embodiments, the cell is an islet. In embodiments, the cell is a 1.1B4 cell. In embodiments, the cell is a human 1.1B4 cell. In embodiments, the cell is a 1.2B4 cell. In embodiments, the cell is a human 1.2B4 cell. In embodiments, the organ is a pancreas. In embodiments, the organ is a human pancreas. In embodiments, the organ is a liver transplanted islet. In embodiments, the organ is a liver transplanted human islet. In embodiments, the organ is a kidney implanted islets. In embodiments, the organ is a kidney implanted human islets. In embodiments, the organ is a skin implanted islet. In embodiments, the organ is a skin implanted human islet. In embodiments, the organ is a muscle implanted islet. In embodiments, the organ is a muscle implanted human islet.

In embodiments, the method further includes detecting a signal emitted by ¹⁸F in the compound. In embodiments, the method further includes generating an image representative of the location and/or amount of the compound based on the signal. In embodiments, the method further includes determining the distribution and/or extent of a disease in the cell, tissue, or organ. In embodiments, the disease is a metabolic disorder. In embodiments, the metabolic disorder is type 1 diabetes. In embodiments, the metabolic disorder is type 2 diabetes.

In an aspect is provided a method of detecting the level of islets in a subject, the method including administering to the subject an effective amount of a compound described herein, or a pharmaceutically acceptable salt thereof. In embodiments, the subject has undergone islet transplantation for treatment of diabetes. In embodiments, the diabetes is type 1 diabetes. In embodiments, the diabetes is type 2 diabetes.

In an aspect is provided a method of detecting the level of beta cells in a subject, the method including administering to the subject an effective amount of a compound described herein, or a pharmaceutically acceptable salt thereof. In embodiments, the subject is undergoing treatment for diabetes. In embodiments, the diabetes is type 1 diabetes. In embodiments, the diabetes is type 2 diabetes. In embodiments, the subject has received an anti-diabetic treatment or potential anti-diabetic treatment. In embodiments, the level of beta cells is the level of endogenous beta cells. In embodiments, the subject has undergone beta cell transplantation for treatment of diabetes.

In an aspect is provided a method of detecting the level of beta cell in a subject, the method including the steps:
(i) administering to the subject an effective amount of a compound described herein, or a pharmaceutically acceptable salt thereof; and
(ii) detecting the level of the compound within the pancreas of the subject.

In embodiments, step (ii) further includes detecting the level of the compound using positron emission tomography.

In embodiments, the compound is a compound of formula (I), (Ia), (I-1), (I-1a), (I-2), (I-2a), (II), (IIa), (III), (IIIa), (IV), (IVa), (IV-1), (IV-1a), (IV-2), or (IV-2a). In embodiments, the compound is a compound of formula (I). In embodiments, the compound is a compound of formula (Ia). In embodiments, the compound is a compound of formula (I-1). In embodiments, the compound is a compound of formula (I-1a). In embodiments, the compound is a compound of formula (I-2). In embodiments, the compound is a compound of formula (I-2a). In embodiments, the compound is a compound of formula (II). In embodiments, the compound is a compound of formula (IIa). In embodiments, the compound is a compound of formula (III). In embodiments, the compound is a compound of formula (IIIa). In embodiments, the compound is a compound of formula (IV). In embodiments, the compound is a compound of formula (IVa). In embodiments, the compound is a compound of formula (IV-1). In embodiments, the compound is a compound of formula (IV-1a). In embodiments, the compound is a compound of formula (IV-2). In embodiments, the compound is a compound of formula (IV-2a).

In embodiments, the method further includes determining whether the subject has a metabolic disorder. In embodiments, the metabolic disorder is diabetes. In embodiments, the diabetes is type 1 diabetes. In embodiments, the diabetes is type 2 diabetes.

### V. Methods of Making

In an aspect is provided a method of making a compound, or a pharmaceutically acceptable salt thereof, having the formula:

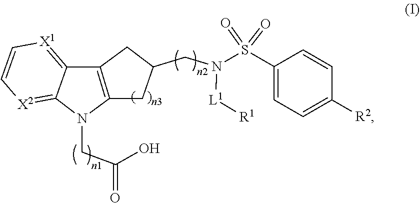

the method including mixing compound (V) and a ¹⁸F fluorinating agent together in a reaction vessel; wherein compound (V) has the formula:

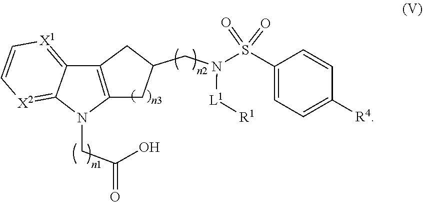

X¹, X², L¹, R¹, R², R⁴, n1, n2, and n3 are as described herein, including in embodiments.

In embodiments, R⁴ is a leaving group and is replaced by a ¹⁸F fluoride anion.

In embodiments, the ¹⁸F fluorinating agent is K¹⁸F.

In embodiments, the method further includes pre-treating the carboxylate group of compound (V) with a cation to make a cationic chelate. In embodiments, the cationic chelate comprises a metal cation and a chelating agent. In embodiments, the metal cation is K⁺. In embodiments, the metal cation is Na⁺. In embodiments, the metal cation is Li⁺. In embodiments, the chelating agent is a cryptand. In embodiments, the cryptand is [2.2.2]-cryptand.

In embodiments, [2.2.2]-cryptand is 1,10-diaza-4,7,13,16,21,24-hexaoxabicyclo[8.8.8]hexacosane. In embodiments [2.2.2]-cryptand has the formula N(CH₂CH₂OCH₂CH₂OCH₂CH₂)₃N. In embodiments, [2.2.2]-cryptand is Kryptofix© 222. In embodiments, [2.2.2]-cryptand has the CAS Number 23978-09-8.

In embodiments, the method further includes pre-treating compound (V) with a salt chelated by a cryptand. In embodiments, the salt chelated by a cryptand is [K([2.2.2]-cryptand)]₂CO₃ and/or [K([2.2.2]-cryptand)]₂C₂O₄. In embodiments, the salt chelated by a cryptand is [K([2.2.2]-cryptand)]₂CO₃ and [K([2.2.2]-cryptand)]₂C₂O₄. In embodiments, the salt chelated by a cryptand is [K([2.2.2]-cryptand)]2CO₃ or [K([2.2.2]-cryptand)]₂C₂O₄. In embodiments, the salt chelated by a cryptand is [K([2.2.2]-cryptand)]₂CO₃. In embodiments, the salt chelated by a cryptand is [K([2.2.2]-cryptand)]₂C₂O₄.

In embodiments, the method further includes mixing compound (V) with a phase transfer catalyst. In embodiments, the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄. In embodiments, the phase transfer catalyst is [K([2.2.2]-cryptand)]₂CO₃. In embodiments, the phase transfer catalyst is [K([2.2.2]-cryptand)]OH. In embodiments, the phase transfer catalyst is [K([2.2.2]-cryptand)]HCO₃. In embodiments, the phase transfer catalyst is [K([2.2.2]-cryptand)]₂C₂O₄. In embodiments, the phase transfer catalyst is ([K+c2.2.2])₂CO₃, [K⁺c2.2.2]OH, [K⁺c2.2.2]HCO₃, or ([K⁺c2.2.2])₂C₂O₄. In embodiments, the phase transfer catalyst is ([K⁺c2.2.2])₂CO₃. In embodiments, the phase transfer catalyst is [K+c2.2.2]OH.

In embodiments, the phase transfer catalyst is [K+c2.2.2]HCO₃. In embodiments, the phase transfer catalyst is ([K⁺c2.2.2])₂C₂O₄.

In embodiments, the method further includes steps described in WO 2015/004029 A1, which is herein incorporated by reference in its entirety for all purposes.

In embodiments, the method further includes mixing compound (V-g) and a methylating agent to make compound (V); wherein compound (V-g) has the formula:

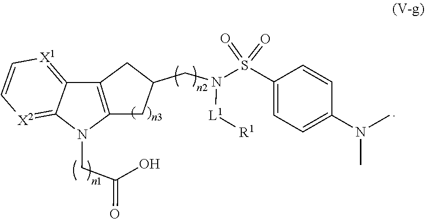

X¹, X², L¹, R¹, n1, n2, and n3 are as described herein, including in embodiments.

In embodiments, the method further includes mixing compound (V-g) and methyl triflate to make compound (V); wherein compound (V-g) has the formula:

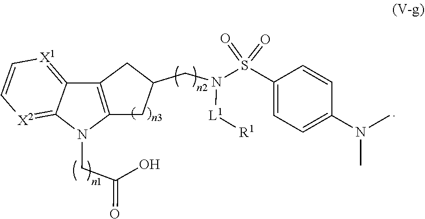

X¹, X², L¹, R¹, n1, n2, and n3 are as described herein, including in embodiments.

In embodiments, compound (V-g) is

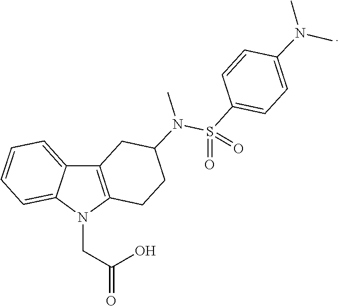 [SMILES: CN(C)c1ccc(S(=O)(=O)N(C)C2CCc3c(c4ccccc4n3CC(=O)O)C2)cc1]

In embodiments, compound (V-g) is

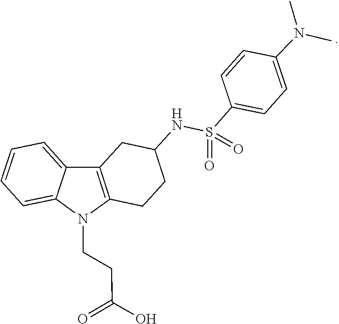 [SMILES: CN(C)c1ccc(S(=O)(=O)NC2CCc3c(c4ccccc4n3CCC(=O)O)C2)cc1]

In embodiments, compound (V-g) is

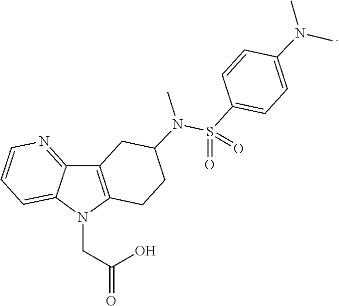 [SMILES: CN(C)c1ccc(S(=O)(=O)N(C)C2CCc3c(c4ncccc4n3CC(=O)O)C2)cc1]

In embodiments, compound (V-g) is

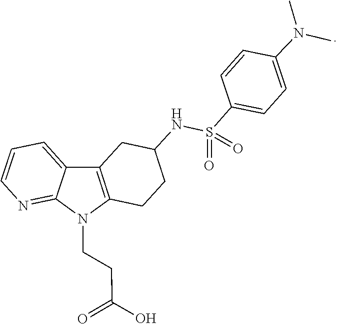 [SMILES: CN(C)c1ccc(S(=O)(=O)NC2CCc3c(c4cccnc4n3CCC(=O)O)C2)cc1]

In embodiments, the method further includes reacting compound (V-f) under methylation conditions to make compound (V-g); wherein compound (V-f) has the formula:

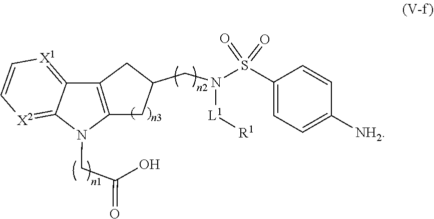

X¹, X², L¹, R¹, n1, n2, and n3 are as described herein, including in embodiments.

In embodiments, the methylation conditions are reductive amination conditions. In embodiments, the reductive amination conditions include a metal hydride. In embodiments, the metal hydride is a boron hydride. In embodiments, the boron hydride is sodium borohydride, sodium cyanoborohydride, or sodium triacetoxyborohydride.

In embodiments, the method further includes mixing compound (V-f), formaldehyde, and sodium cyanoborohydride to make compound (V-g); wherein compound (V-f) has the formula:

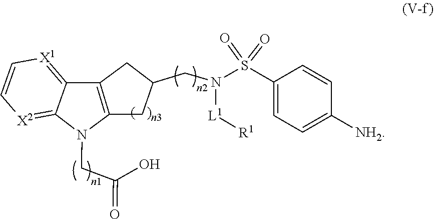

X¹, X², L¹, R¹, n1, n2, and n3 are as described herein, including in embodiments.

In embodiments, compound (V-f) is

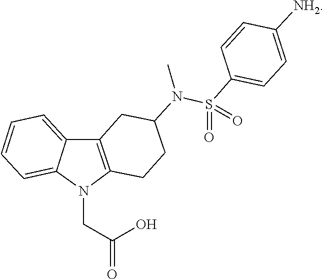 [SMILES: CN(C1CCc2c(c3ccccc3n2CC(=O)O)C1)S(=O)(=O)c1ccc(N)cc1]

In embodiments, compound (V-f) is

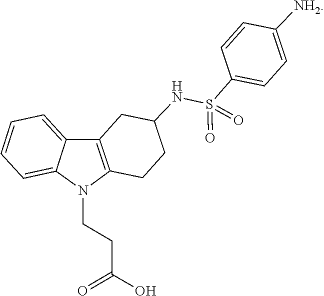 [SMILES: Nc1ccc(S(=O)(=O)NC2CCc3c(c4ccccc4n3CCC(=O)O)C2)cc1]

In embodiments, compound (V-f) is

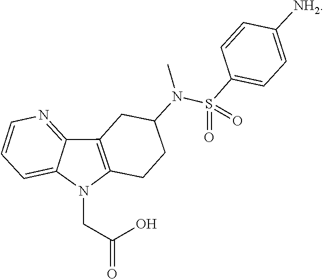 [SMILES: CN(C1CCc2c(c3ncccc3n2CC(=O)O)C1)S(=O)(=O)c1ccc(N)cc1]

In embodiments, compound (V-f) is

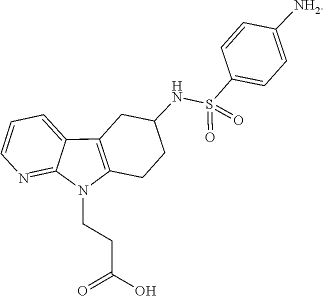 [SMILES: Nc1ccc(S(=O)(=O)NC2CCc3c(c4cccnc4n3CCC(=O)O)C2)cc1]

In embodiments, the method further includes reacting compound (V-e) under reducing conditions to make compound (V-f); wherein compound (V-e) has the formula:

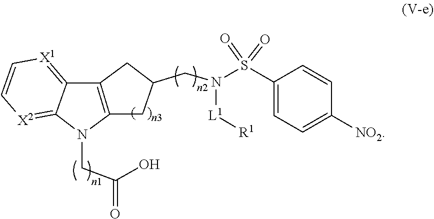

X¹, X², L¹, R¹, n1, n2, and n3 are as described herein, including in embodiments.

In embodiments, the reducing conditions are catalytic hydrogenation conditions. In embodiments, the catalytic hydrogenation conditions include a metal catalyst. In embodiments, the metal catalyst is Raney nickel, palladium-on-carbon, platinum(IV) oxide, or Urushibara nickel. In embodiments, the reducing conditions include zinc. In embodiments, the reducing conditions include iron in acidic media. In embodiments, the reducing conditions include sodium hydrosulfite. In embodiments, the reducing conditions include sodium sulfide or hydrogen sulfide. In embodiments, the reducing conditions include tin(II) chloride. In embodiments, the reducing conditions include titanium(III) chloride. In embodiments, the reducing conditions include samarium. In embodiments, the reducing conditions include hydroiodic acid.

In embodiments, the method further includes mixing compound (V-e), zinc, and ammonium chloride to make compound (V-f); wherein compound (V-e) has the formula:

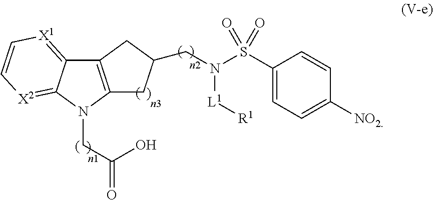

X¹, X², L¹, R¹, n1, n2, and n3 are as described herein, including in embodiments.

In embodiments, compound (V-e) is

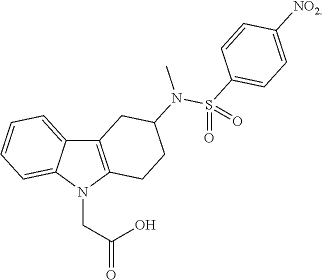 [SMILES: CN(C1CCc2c(c3ccccc3n2CC(=O)O)C1)S(=O)(=O)c1ccc([N+](=O)[O-])cc1]

In embodiments, compound (V-e) is

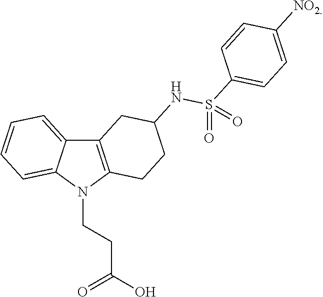 [SMILES: O=C(O)CCn1c2c(c3ccccc31)CC(NS(=O)(=O)c1ccc([N+](=O)[O-])cc1)CC2]

In embodiments, compound (V-e) is

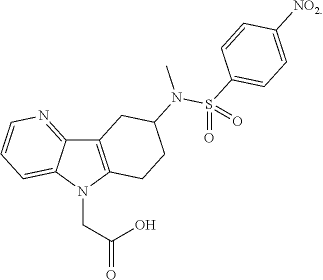 [SMILES: CN(C1CCc2c(c3ncccc3n2CC(=O)O)C1)S(=O)(=O)c1ccc([N+](=O)[O-])cc1]

In embodiments, compound (V-e) is

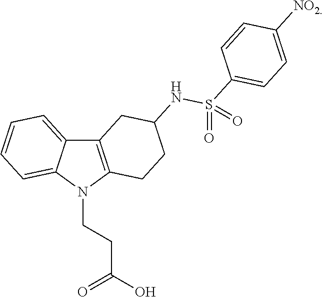 [SMILES: O=C(O)CCn1c2c(c3ccccc31)CC(NS(=O)(=O)c1ccc([N+](=O)[O-])cc1)CC2]

In embodiments, the method further includes reacting compound (V-d) under hydrolysis conditions to make compound (V-e); wherein compound (V-d) has the formula:

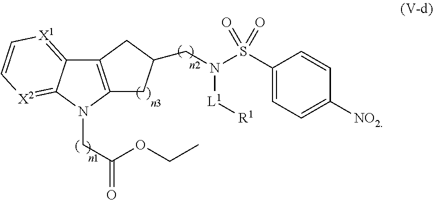

X¹, X², L¹, R¹, n1, n2, and n3 are as described herein, including in embodiments.

In embodiments, the hydrolysis conditions are basic hydrolysis conditions. In embodiments, the basic hydrolysis conditions include lithium hydroxide or sodium hydroxide. In embodiments, the hydrolysis conditions are acidic hydrolysis conditions. In embodiments, the acidic hydrolysis conditions include sulfuric acid.

In embodiments, the method further includes mixing compound (V-d) and lithium hydroxide to make compound (V-e); wherein compound (V-d) has the formula:

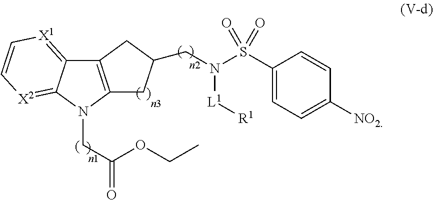

X¹, X², L¹, R¹, n1, n2, and n3 are as described herein, including in embodiments.

In embodiments, compound (V-d) is

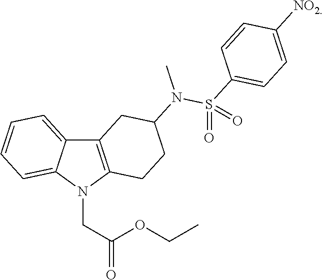 [SMILES: CCOC(=O)Cn1c2c(c3ccccc31)CC(N(C)S(=O)(=O)c1ccc([N+](=O)[O-])cc1)CC2]

In embodiments, compound (V-d) is

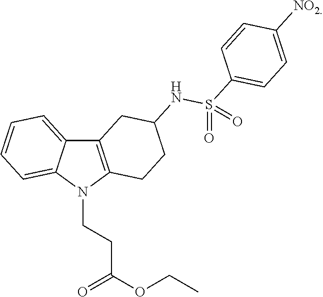 [SMILES: CCOC(=O)CCn1c2c(c3ccccc31)CC(NS(=O)(=O)c1ccc([N+](=O)[O-])cc1)CC2]

In embodiments, compound (V-d) is

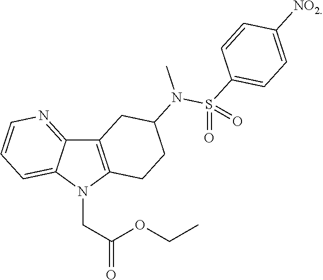 [SMILES: CCOC(=O)Cn1c2c(c3ncccc31)CC(N(C)S(=O)(=O)c1ccc([N+](=O)[O-])cc1)CC2]

In embodiments, compound (V-d) is

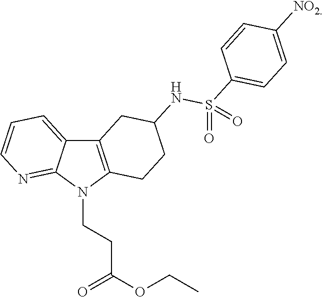 [SMILES: CCOC(=O)CCn1c2c(c3cccnc31)CC(NS(=O)(=O)c1ccc([N+](=O)[O-])cc1)CC2]

In embodiments, the method further includes mixing compound (V-c), compound (c1), and a base to make compound (V-d); wherein compound (V-c) has the formula:

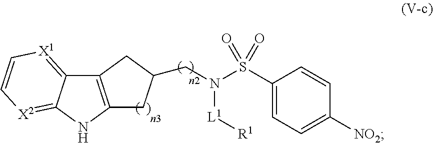

compound (c1) has the formula:

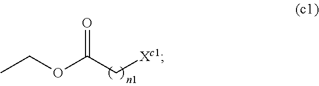

and X^{c1} is a halogen. X¹, X², L¹, R¹, n1, n2, and n3 are as described herein, including in embodiments.

In embodiments, X^{c1} is —Br. In embodiments, X^{c1} is —Cl. In embodiments, X^{c1} is —I.

In embodiments, compound (c1) is

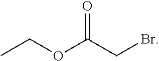

In embodiments, compound (c1) is

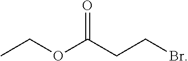

In embodiments, the method further includes mixing compound (V-c), compound (c1), and sodium hydride to make compound (V-d); wherein compound (V-c) has the formula:

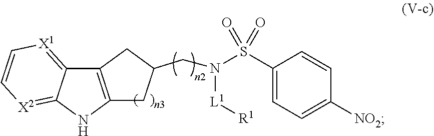

compound (c1) has the formula:

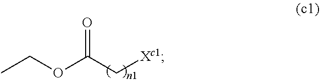

and X^{c1} is a halogen. X¹, X², L¹, R¹, n1, n2, and n3 are as described herein, including in embodiments.

In embodiments, compound (V-c) is

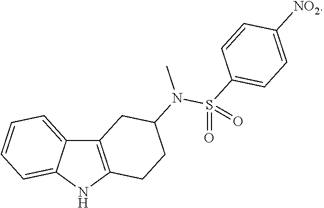 [SMILES: CN(C1CCc2[nH]c3ccccc3c2C1)S(=O)(=O)c1ccc([N+](=O)[O-])cc1]

In embodiments, compound (V-c) is

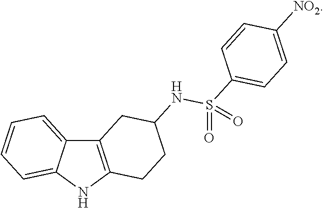 [SMILES: O=[N+]([O-])c1ccc(S(=O)(=O)NC2CCc3[nH]c4ccccc4c3C2)cc1]

In embodiments, compound (V-c) is

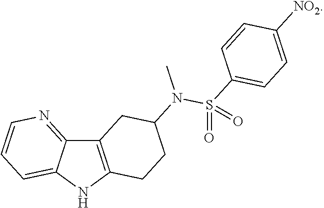 [SMILES: CN(C1CCc2[nH]c3cccnc3c2C1)S(=O)(=O)c1ccc([N+](=O)[O-])cc1]

In embodiments, compound (V-c) is

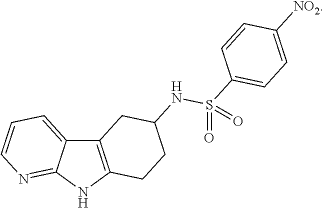 [SMILES: O=[N+]([O-])c1ccc(S(=O)(=O)NC2CCc3[nH]c4ncccc4c3C2)cc1]

In embodiments, the method further includes mixing

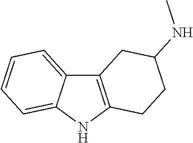

and 4-nitrobenzenesulfonyl chloride to form

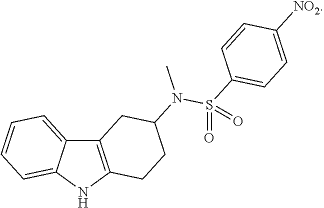 [SMILES: CN(C1CCc2[nH]c3ccccc3c2C1)S(=O)(=O)c1ccc([N+](=O)[O-])cc1]

In embodiments, the method further includes reacting

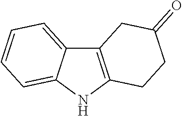 [SMILES: O=C1CCc2[nH]c3ccccc3c2C1]

under reductive amination conditions to form

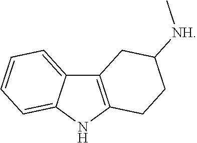

In embodiments, the method further includes mixing

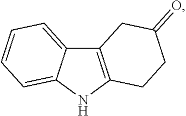 [SMILES: O=C1CCc2[nH]c3ccccc3c2C1]

methylamine, and sodium cyanoborohydride to form

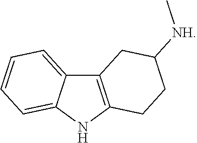

In an aspect is provided a method of making a compound, or a pharmaceutically acceptable salt thereof, having the formula:

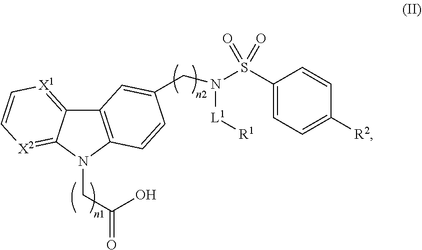

the method including mixing compound (VI) and a ¹⁸F fluorinating agent together in a reaction vessel; wherein compound (VI) has the formula:

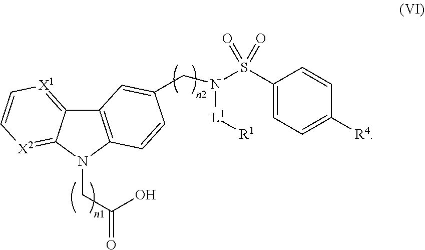

X¹, X², L¹, R¹, R², R⁴, n1, and n2 are as described herein, including in embodiments.

In embodiments, the method further includes pre-treating the carboxylate group of compound (VI) with a cation to make a cationic chelate. In embodiments, the cationic chelate comprises a metal cation and a chelating agent. In embodiments, the metal cation is K⁺. In embodiments, the metal cation is Na⁺. In embodiments, the metal cation is Li⁺. In embodiments, the chelating agent is a cryptand. In embodiments, the cryptand is [2.2.2]-cryptand.

In embodiments, the method further includes pre-treating compound (VI) with a salt chelated by a cryptand. In embodiments, the salt chelated by a cryptand is [K([2.2.2]-cryptand)]₂CO₃ and/or [K([2.2.2]-cryptand)]₂C₂O₄. In embodiments, the salt chelated by a cryptand is [K([2.2.2]-cryptand)]₂CO₃ and [K([2.2.2]-cryptand)]₂C₂O₄. In embodiments, the salt chelated by a cryptand is [K([2.2.2]-cryptand)]₂CO₃ or [K([2.2.2]-cryptand)]₂C₂O₄. In embodiments, the salt chelated by a cryptand is [K([2.2.2]-cryptand)]₂CO₃. In embodiments, the salt chelated by a cryptand is [K([2.2.2]-cryptand)]₂C₂O₄.

In embodiments, the method further includes mixing compound (VI) with a phase transfer catalyst. In embodiments, the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄. In embodiments, the phase transfer catalyst is [K([2.2.2]-cryptand)]₂CO₃. In embodiments, the phase transfer catalyst is [K([2.2.2]-cryptand)]OH. In embodiments, the phase transfer catalyst is [K([2.2.2]-cryptand)]HCO₃. In embodiments, the phase transfer catalyst is [K([2.2.2]-cryptand)]₂C₂O₄. In embodiments, the phase transfer catalyst is ([K+c2.2.2])₂CO₃, [K⁺c2.2.2]OH, [K⁺c2.2.2]HCO₃, or ([K⁺c2.2.2])₂C₂O₄. In embodiments, the phase transfer catalyst is ([K⁺c2.2.2])₂CO₃. In embodiments, the phase transfer catalyst is [K⁺c2.2.2]OH. In embodiments, the phase transfer catalyst is [K+c2.2.2]HCO₃. In embodiments, the phase transfer catalyst is ([K⁺c2.2.2])₂C₂O₄.

In embodiments, the method further includes mixing compound (VI-g) and a methylating agent to make compound (VI); wherein compound (VI-g) has the formula:

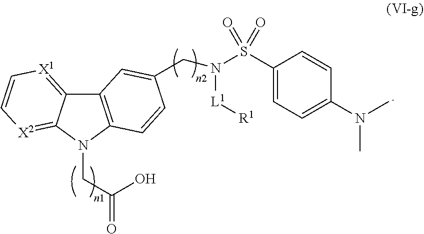

X¹, X², L¹, R¹, n1, and n2 are as described herein, including in embodiments.

In embodiments, the method further includes mixing compound (VI-g) and methyl triflate to make compound (VI); wherein compound (VI-g) has the formula:

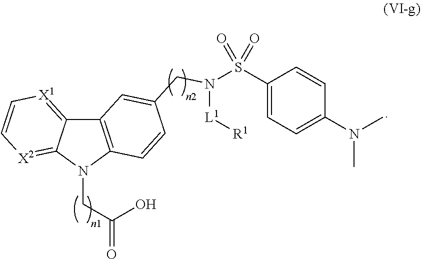

X¹, X², L¹, R¹, n1, and n2 are as described herein, including in embodiments.

In embodiments, compound (VI-g) is

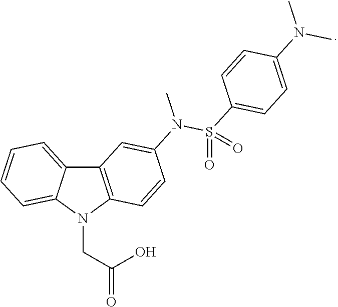 [SMILES: CN(C)c1ccc(S(=O)(=O)N(C)c2ccc3c(c2)c2ccccc2n3CC(=O)O)cc1]

In embodiments, the method further includes reacting compound (VI-f) under methylation conditions to make compound (VI-g); wherein compound (VI-f) has the formula:

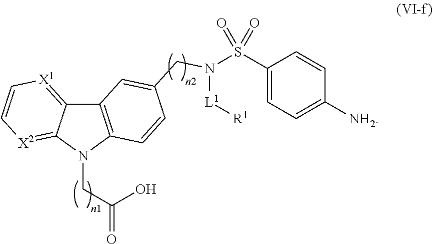

X¹, X², L¹, R¹, n1, and n2 are as described herein, including in embodiments. In embodiments, the methylation conditions are as described herein, including in embodiments.

In embodiments, the method further includes mixing compound (VI-f), formaldehyde, and sodium cyanoborohydride to make compound (VI-g); wherein compound (VI-f) has the formula:

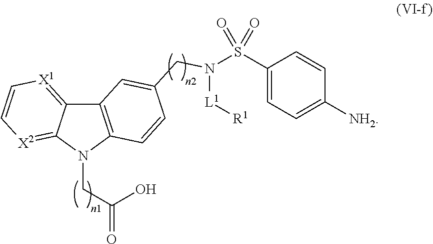

X¹, X², L¹, R¹, n1, and n2 are as described herein, including in embodiments.

In embodiments, compound (VI-f) is

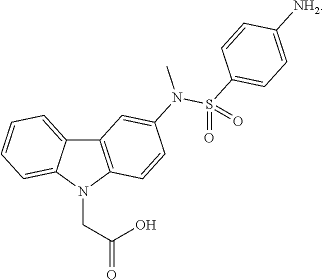 [SMILES: CN(c1ccc2c(c1)c1ccccc1n2CC(=O)O)S(=O)(=O)c1ccc(N)cc1]

In embodiments, the method further includes reacting compound (VI-e) under reducing conditions to make compound (VI-f); wherein compound (VI-e) has the formula:

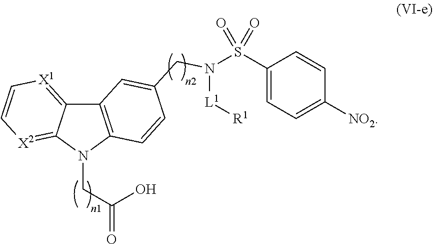

X¹, X², L¹, R¹, n1, and n2 are as described herein, including in embodiments. In embodiments, the reducing conditions are as described herein, including in embodiments.

In embodiments, the method further includes mixing compound (VI-e), zinc, and ammonium chloride to make compound (VI-f); wherein compound (VI-e) has the formula:

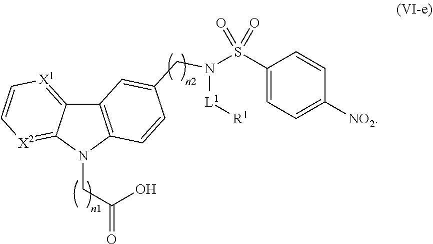

X¹, X², L¹, R¹, n1, and n2 are as described herein, including in embodiments.

In embodiments, compound (VI-e) is

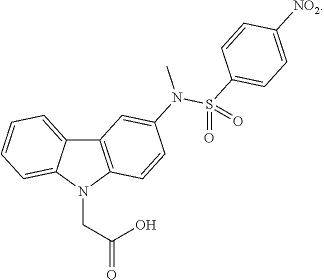 [SMILES: CN(c1ccc2c(c1)c1ccccc1n2CC(=O)O)S(=O)(=O)c1ccc([N+](=O)[O-])cc1]

In embodiments, the method further includes reacting compound (VI-d) under hydrolysis conditions to make compound (VI-e); wherein compound (VI-d) has the formula:

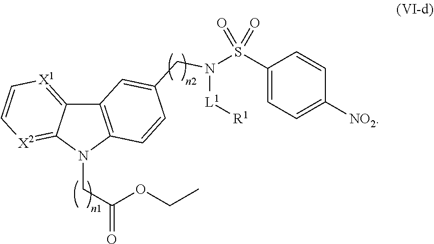

X¹, X², L¹, R¹, n1, and n2 are as described herein, including in embodiments. In embodiments, the hydrolysis conditions are as described herein, including in embodiments.

In embodiments, the method further includes mixing compound (VI-d) and lithium hydroxide to make compound (VI-e); wherein compound (VI-d) has the formula:

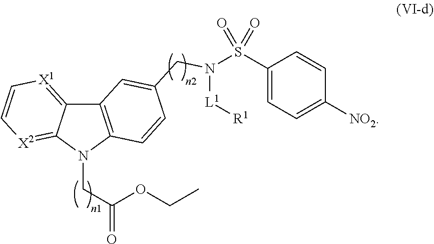

X¹, X², L¹, R¹, n1, and n2 are as described herein, including in embodiments.

In embodiments, compound (VI-d) is

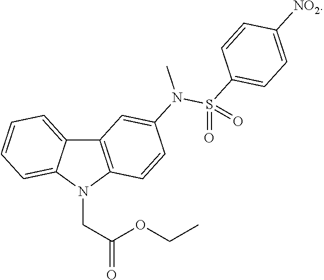 [SMILES: CCOC(=O)Cn1c2ccccc2c2cc(N(C)S(=O)(=O)c3ccc([N+](=O)[O-])cc3)ccc21]

In embodiments, the method further includes mixing compound (VI-c), compound (c1), and a base to make compound (VI-d); wherein compound (VI-c) has the formula:

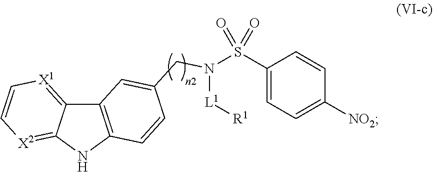

compound (c1) has the formula:

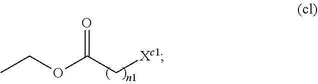

n1 (c1) and X^{c1} is a halogen. X¹, X², L¹, R¹, n1, and n2 are as described herein, including in embodiments.

In embodiments, the method further includes mixing compound (VI-c), compound (c1), and sodium hydride to make compound (VI-d); wherein compound (VI-c) has the formula:

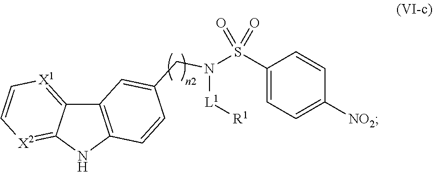

compound (c1) has the formula:

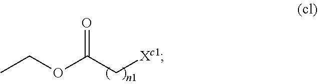

and X^{c1} is a halogen. X¹, X², L¹, R¹, n1, and n2 are as described herein, including in embodiments.

In embodiments, compound (VI-c) is

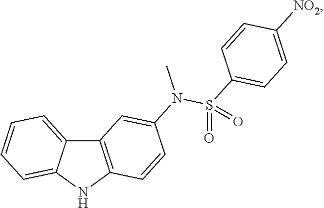 [SMILES: CN(c1ccc2[nH]c3ccccc3c2c1)S(=O)(=O)c1ccc([N+](=O)[O-])cc1]

In an aspect is provided a method of making a compound, or a pharmaceutically acceptable salt thereof, having the formula:

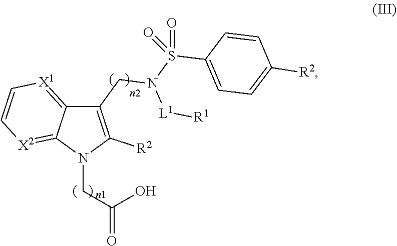

the method including mixing compound (VII) and a ¹⁸F fluorinating agent together in a reaction vessel; wherein compound (VII) has the formula:

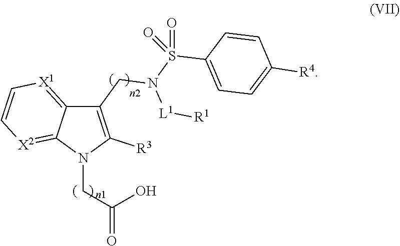

X¹, X², L¹, R¹, R², R³, R⁴, n1, and n2 are as described herein, including in embodiments.

In embodiments, the method further includes pre-treating the carboxylate group of compound (VII) with a cation to make a cationic chelate. In embodiments, the cationic chelate comprises a metal cation and a chelating agent. In embodiments, the metal cation is K⁺. In embodiments, the metal cation is Na⁺. In embodiments, the metal cation is Li⁺. In embodiments, the chelating agent is a cryptand. In embodiments, the cryptand is [2.2.2]-cryptand.

In embodiments, the method further includes pre-treating compound (VII) with a salt chelated by a cryptand. In embodiments, the salt chelated by a cryptand is [K([2.2.2]-cryptand)]₂CO₃ and/or [K([2.2.2]-cryptand)]₂C₂O₄. In embodiments, the salt chelated by a cryptand is [K([2.2.2]-cryptand)]₂CO₃ and [K([2.2.2]-cryptand)]₂C₂O₄. In embodiments, the salt chelated by a cryptand is [K([2.2.2]-cryptand)]₂CO₃ or [K([2.2.2]-cryptand)]₂C₂O₄. In embodiments, the salt chelated by a cryptand is [K([2.2.2]-cryptand)]₂CO₃. In embodiments, the salt chelated by a cryptand is [K([2.2.2]-cryptand)]₂C₂O₄.

In embodiments, the method further includes mixing compound (VII) with a phase transfer catalyst. In embodiments, the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄. In embodiments, the phase transfer catalyst is [K([2.2.2]-cryptand)]₂CO₃. In embodiments, the phase transfer catalyst is [K([2.2.2]-cryptand)]OH. In embodiments, the phase transfer catalyst is [K([2.2.2]-cryptand)]HCO₃. In embodiments, the phase transfer catalyst is [K([2.2.2]-cryptand)]₂C₂O₄. In embodiments, the phase transfer catalyst is ([K⁺c2.2.2])₂CO₃, [K⁺c2.2.2]OH, [K⁺c2.2.2]HCO₃, or ([K⁺c2.2.2])₂C₂O₄. In embodiments, the phase transfer catalyst is ([K⁺c2.2.2])₂CO₃. In embodiments, the phase transfer catalyst is [K+c2.2.2]OH. In embodiments, the phase transfer catalyst is [K⁺c2.2.2]HCO₃. In embodiments, the phase transfer catalyst is ([K⁺c2.2.2])₂C₂O₄.

In embodiments, the method further includes mixing compound (VII-g) and a methylating agent to make compound (VII); wherein compound (VII-g) has the formula:

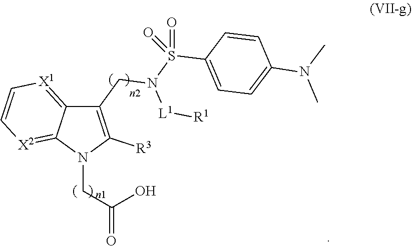

X¹, X², L¹, R¹, R³, n1, and n2 are as described herein, including in embodiments.

In embodiments, the method further includes mixing compound (VII-g) and methyl triflate to make compound (VII); wherein compound (VII-g) has the formula:

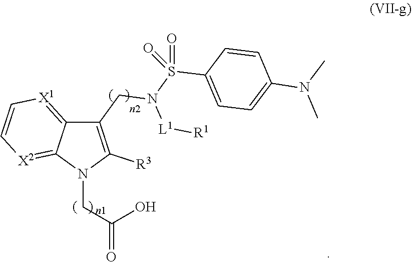

X¹, X², L¹, R¹, R³, n1, and n2 are as described herein, including in embodiments.

In embodiments, compound (VII-g) is

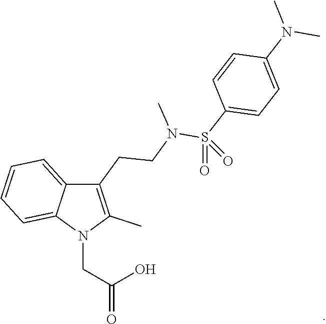 [SMILES: Cc1c(CCN(C)S(=O)(=O)c2ccc(N(C)C)cc2)c2ccccc2n1CC(=O)O]

In embodiments, compound (VII-g) is

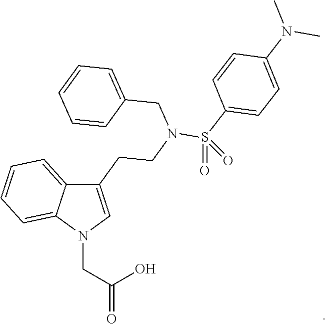 [SMILES: CN(C)c1ccc(S(=O)(=O)N(CCc2cn(CC(=O)O)c3ccccc23)Cc2ccccc2)cc1]

In embodiments, the method further includes reacting compound (VII-f) under methylation conditions to make compound (VII-g); wherein compound (VII-f) has the formula:

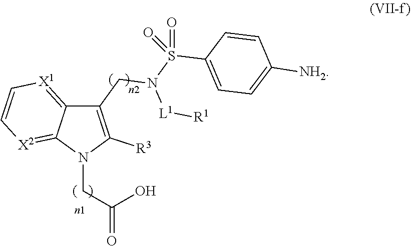

X¹, X², L¹, R¹, R³, n1, and n2 are as described herein, including in embodiments. In embodiments, the methylation conditions are as described herein, including in embodiments.

In embodiments, the method further includes mixing compound (VII-f), formaldehyde, and sodium cyanoborohydride to make compound (VII-g); wherein compound (VII-f) has the formula:

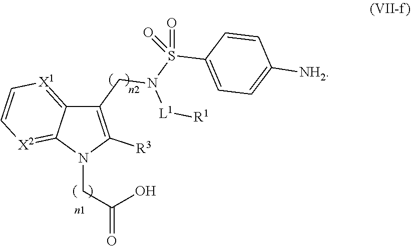

X¹, X², L¹, R¹, R³, n1, and n2 are as described herein, including in embodiments.

In embodiments, compound (VII-f) is

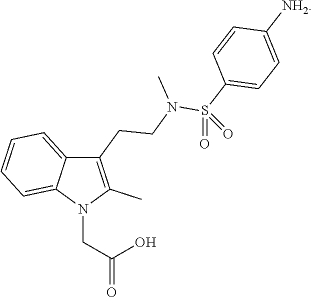 [SMILES: Cc1c(CCN(C)S(=O)(=O)c2ccc(N)cc2)c2ccccc2n1CC(=O)O]

In embodiments, compound (VII-f) is

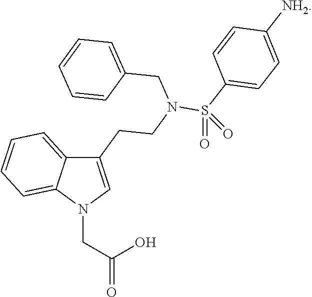 [SMILES: Nc1ccc(S(=O)(=O)N(CCc2cn(CC(=O)O)c3ccccc23)Cc2ccccc2)cc1]

In embodiments, the method further includes reacting compound (VII-e) under reducing conditions to make compound (VII-f); wherein compound (VII-e) has the formula:

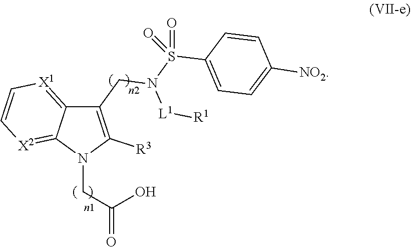

X¹, X², L¹, R¹, R³, n1, and n2 are as described herein, including in embodiments. In embodiments, the reducing conditions are as described herein, including in embodiments.

In embodiments, the method further includes mixing compound (VII-e), zinc, and ammonium chloride to make compound (VII-f); wherein compound (VII-e) has the formula:

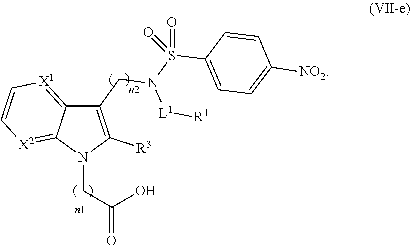

X¹, X², L¹, R¹, R³, n1, and n2 are as described herein, including in embodiments.

In embodiments, compound (VII-e) is

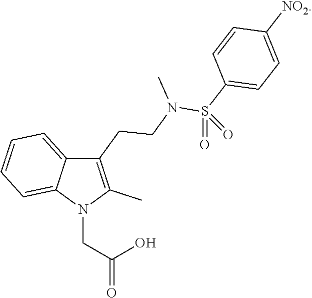 [SMILES: Cc1c(CCN(C)S(=O)(=O)c2ccc([N+](=O)[O-])cc2)c2ccccc2n1CC(=O)O]

In embodiments, compound (VII-e) is

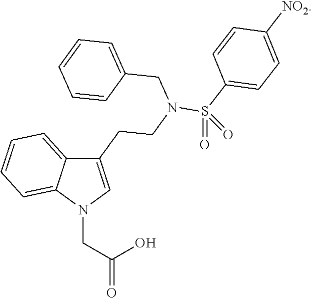 [SMILES: O=C(O)Cn1cc(CCN(Cc2ccccc2)S(=O)(=O)c2ccc([N+](=O)[O-])cc2)c2ccccc21]

In embodiments, the method further includes reacting compound (VII-d) under hydrolysis conditions to make compound (VII-e); wherein compound (VII-d) has the formula:

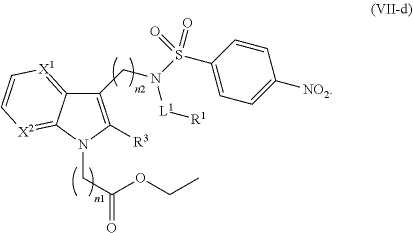

X¹, X², L¹, R¹, R³, n1, and n2 are as described herein, including in embodiments. In embodiments, the hydrolysis conditions are as described herein, including in embodiments.

In embodiments, the method further includes mixing compound (VII-d) and lithium hydroxide to make compound (VII-e); wherein compound (VII-d) has the formula:

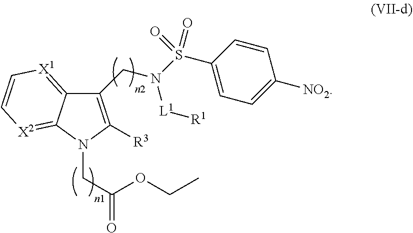

X¹, X², L¹, R¹, R³, n1, and n2 are as described herein, including in embodiments.

In embodiments, compound (VII-d) is

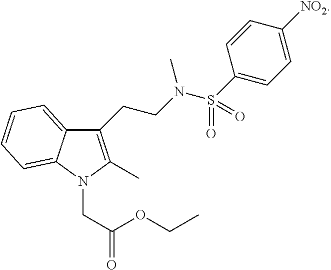 [SMILES: CCOC(=O)Cn1c(C)c(CCN(C)S(=O)(=O)c2ccc([N+](=O)[O-])cc2)c2ccccc21]

In embodiments, compound (VII-d) is

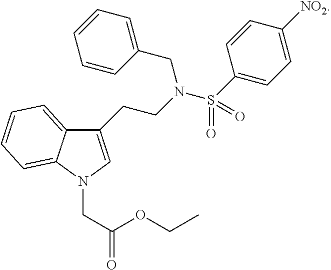 [SMILES: CCOC(=O)Cn1cc(CCN(Cc2ccccc2)S(=O)(=O)c2ccc([N+](=O)[O-])cc2)c2ccccc21]

In embodiments, the method further includes mixing compound (VII-c), compound (c1), and a base to make compound (VII-d); wherein compound (VII-c) has the formula:

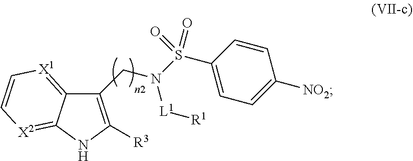

compound (c1) has the formula

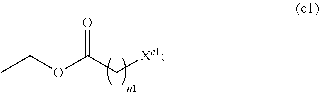

and X^{c1} is a halogen. X¹, X², L¹, R¹, R³, n1, and n2 are as described herein, including in embodiments.

In embodiments, the method further includes mixing compound (VII-c), compound (c1), and sodium hydride to make compound (VII-d); wherein compound (VII-c) has the formula:

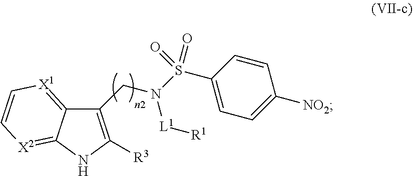

compound (c1) has the formula:

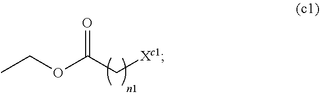

and X^{c1} is a halogen. X¹, X², L¹, R¹, R³, n1, and n2 are as described herein, including in embodiments.

In embodiments, compound (VII-c) is

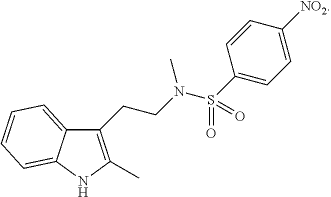 [SMILES: Cc1[nH]c2ccccc2c1CCN(C)S(=O)(=O)c1ccc([N+](=O)[O-])cc1]

In embodiments, compound (VII-c) is

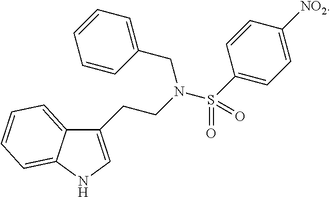 [SMILES: O=[N+]([O-])c1ccc(S(=O)(=O)N(CCc2c[nH]c3ccccc23)Cc2ccccc2)cc1]

In embodiments, the method further includes mixing

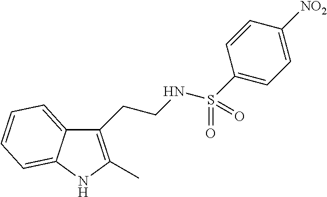 [SMILES: Cc1[nH]c2ccccc2c1CCNS(=O)(=O)c1ccc([N+](=O)[O-])cc1]

and an alkylating agent to form

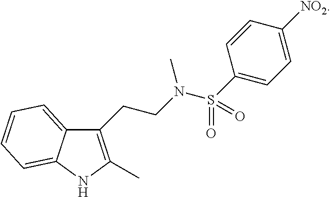 [SMILES: Cc1[nH]c2ccccc2c1CCN(C)S(=O)(=O)c1ccc([N+](=O)[O-])cc1]

In embodiments, the alkylating agent is methyl iodide. In embodiments, the method further includes mixing

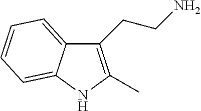

and 4-nitrobenzenesulfonyl chloride to form

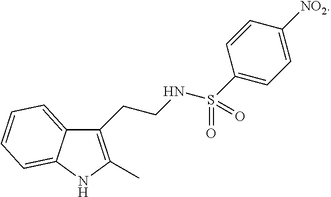 [SMILES: Cc1[nH]c2ccccc2c1CCNS(=O)(=O)c1ccc([N+](=O)[O-])cc1]

In embodiments, the method further includes mixing

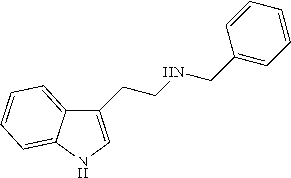 [SMILES: c1ccc(CNCCc2c[nH]c3ccccc23)cc1]

and 4-nitrobenzenesulfonyl chloride to form

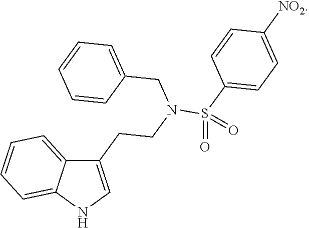 [SMILES: O=[N+]([O-])c1ccc(S(=O)(=O)N(CCc2c[nH]c3ccccc23)Cc2ccccc2)cc1]

In embodiments, the method further includes reacting

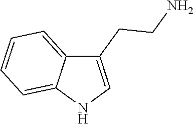 [SMILES: NCCc1c[nH]c2ccccc12]

under reductive amination conditions to form

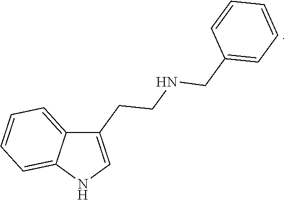 [SMILES: c1ccc(CNCCc2c[nH]c3ccccc23)cc1]

In embodiments, the method further includes mixing

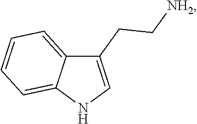 [SMILES: NCCc1c[nH]c2ccccc12]

benzaldehyde, and sodium cyanoborohydride to form

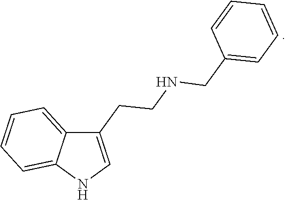 [SMILES: c1ccc(CNCCc2c[nH]c3ccccc23)cc1]

In an aspect is provided a method of making a compound, or a pharmaceutically acceptable salt thereof, having the formula:

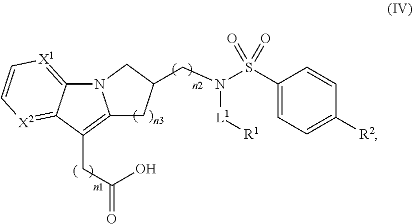

the method including mixing compound (VIII) and a ¹⁸F fluorinating agent together in a reaction vessel; wherein compound (VIII) has the formula:

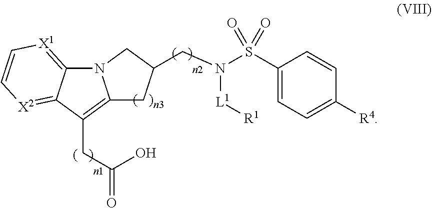

X¹, X², L¹, R¹, R², R⁴, n1, n2, and n3 are as described herein, including in embodiments.

In embodiments, the method further includes pre-treating the carboxylate group of compound (VIII) with a cation to make a cationic chelate. In embodiments, the cationic chelate comprises a metal cation and a chelating agent. In embodiments, the metal cation is K⁺. In embodiments, the metal cation is Na⁺. In embodiments, the metal cation is Li⁺. In embodiments, the chelating agent is a cryptand. In embodiments, the cryptand is [2.2.2]-cryptand.

In embodiments, the method further includes pre-treating compound (VIII) with a salt chelated by a cryptand. In embodiments, the salt chelated by a cryptand is [K([2.2.2]-cryptand)]₂CO₃ and/or [K([2.2.2]-cryptand)]₂C₂O₄. In embodiments, the salt chelated by a cryptand is [K([2.2.2]-cryptand)]₂CO₃ and [K([2.2.2]-cryptand)]₂C₂O₄. In embodiments, the salt chelated by a cryptand is [K([2.2.2]-cryptand)]₂CO₃ or [K([2.2.2]-cryptand)]₂C₂O₄. In embodiments, the salt chelated by a cryptand is [K([2.2.2]-cryptand)]₂CO₃. In embodiments, the salt chelated by a cryptand is [K([2.2.2]-cryptand)]₂C₂O₄.

In embodiments, the method further includes mixing compound (VIII) with a phase transfer catalyst. In embodiments, the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄. In embodiments, the phase transfer catalyst is [K([2.2.2]-cryptand)]₂CO₃. In embodiments, the phase transfer catalyst is [K([2.2.2]-cryptand)]OH. In embodiments, the phase transfer catalyst is [K([2.2.2]-cryptand)]HCO₃. In embodiments, the phase transfer catalyst is [K([2.2.2]-cryptand)]₂C₂O₄. In embodiments, the phase transfer catalyst is ([K+c2.2.2])₂CO₃, [K⁺c2.2.2]OH, [K⁺c2.2.2]HCO₃, or ([K⁺c2.2.2])₂C₂O₄. In embodiments, the phase transfer catalyst is ([K⁺c2.2.2])₂CO₃. In embodiments, the phase transfer catalyst is [K+c2.2.2]OH. In embodiments, the phase transfer catalyst is [K+c2.2.2]HCO₃. In embodiments, the phase transfer catalyst is ([K⁺c2.2.2])₂C₂O₄.

In embodiments, the method further includes mixing compound (VIII-g) and a methylating agent to make compound (VIII); wherein compound (VIII-g) has the formula:

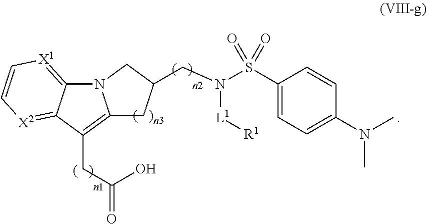

X¹, X², L, R¹, n1, n2, and n3 are as described herein, including in embodiments.

In embodiments, the method further includes mixing compound (VIII-g) and methyl triflate to make compound (VIII); wherein compound (VIII-g) has the formula:

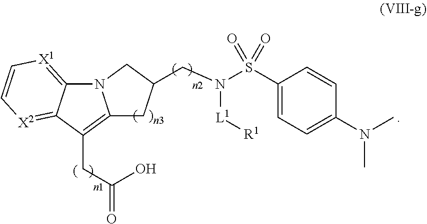

X¹, X², L¹, R¹, n1, n2, and n3 are as described herein, including in embodiments.

In embodiments, compound (VIII-g) is

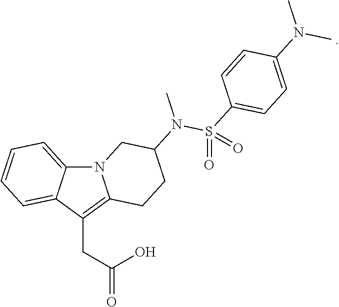 [SMILES: CN(C)c1ccc(S(=O)(=O)N(C)C2CCc3c(CC(=O)O)c4ccccc4n3C2)cc1]

In embodiments, the method further includes reacting compound (VIII-f) under methylation conditions to make compound (VIII-g); wherein compound (VIII-f) has the formula:

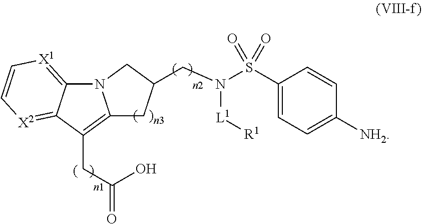

X¹, X², L¹, R¹, n1, n2, and n3 are as described herein, including in embodiments. In embodiments, the methylation conditions are as described herein, including in embodiments.

In embodiments, the method further includes mixing compound (VIII-f), formaldehyde, and sodium cyanoborohydride to make compound (VIII-g); wherein compound (VIII-f) has the formula:

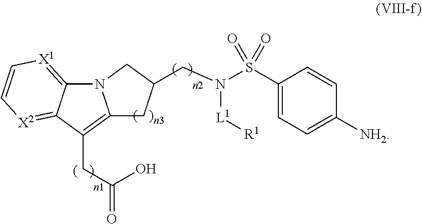

X¹, X², L¹, R¹, n1, n2, and n3 are as described herein, including in embodiments.

In embodiments, compound (VIII-f) is

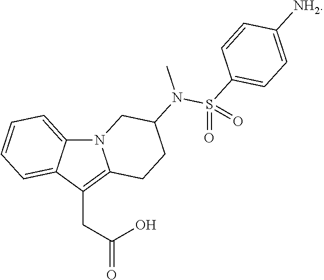 [SMILES: CN(C1CCc2c(CC(=O)O)c3ccccc3n2C1)S(=O)(=O)c1ccc(N)cc1]

In embodiments, the method further includes reacting compound (VIII-e) under reducing conditions to make compound (VIII-f); wherein compound (VIII-e) has the formula:

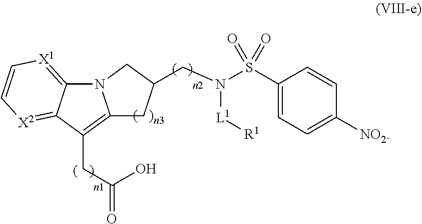

X¹, X², L¹, R¹, n1, n2, and n3 are as described herein, including in embodiments. In embodiments, the reducing conditions are as described herein, including in embodiments.

In embodiments, the method further includes mixing compound (VIII-e), zinc, and ammonium chloride to make compound (VIII-f); wherein compound (VIII-e) has the formula:

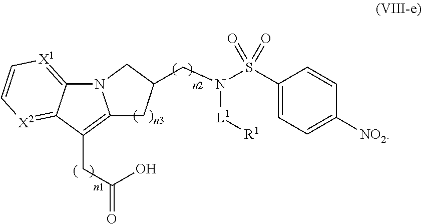

X¹, X², L¹, R¹, n1, n2, and n3 are as described herein, including in embodiments.

In embodiments, compound (VIII-e) is

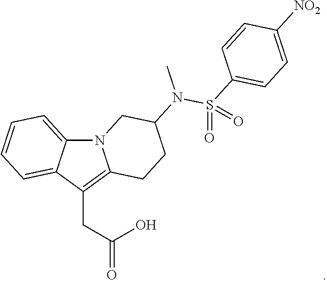 [SMILES: CN(C1CCc2c(CC(=O)O)c3ccccc3n2C1)S(=O)(=O)c1ccc([N+](=O)[O-])cc1]

In embodiments, the method further includes reacting compound (VIII-d) under hydrolysis conditions to make compound (VIII-e); wherein compound (VIII-d) has the formula:

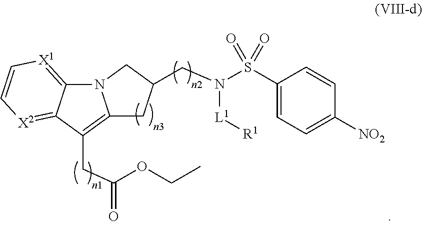

X¹, X², L¹, R¹, n1, n2, and n3 are as described herein, including in embodiments. In embodiments, the hydrolysis conditions are as described herein, including in embodiments.

In embodiments, the method further includes mixing compound (VIII-d) and lithium hydroxide to make compound (VIII-e); wherein compound (VIII-d) has the formula:

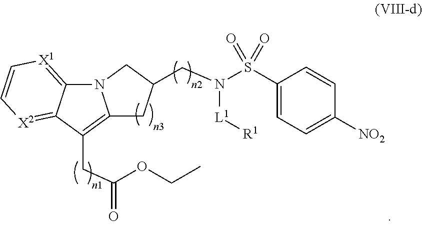

X¹, X², L¹, R¹, n1, n2, and n3 are as described herein, including in embodiments.

In embodiments, compound (VIII-d) is

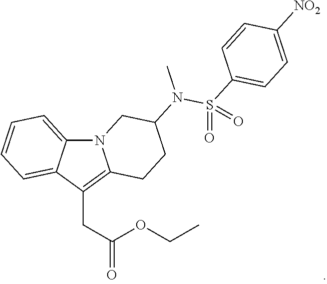 [SMILES: CCOC(=O)Cc1c2n(c3ccccc13)CC(N(C)S(=O)(=O)c1ccc([N+](=O)[O-])cc1)CC2]

In embodiments, the method further includes mixing compound (VIII-c), compound (c1), and a base to make compound (VIII-d); wherein compound (VIII-c) has the formula:

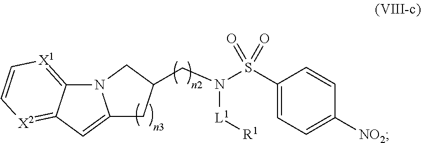

compound (c1) has the formula:

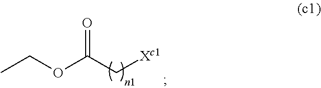

and X^{c1} is a halogen. X¹, X², L¹, R¹, n1, n2, and n3 are as described herein, including in embodiments.

In embodiments, the method further includes mixing compound (VIII-c), compound (c1), and sodium hydride to make compound (VIII-d); wherein compound (VIII-c) has the formula:

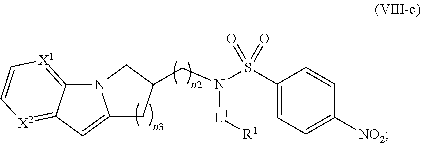

compound (c1) has the formula:

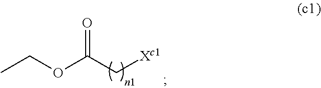

and X^{c1} is a halogen. X¹, X², L¹, R¹, n1, n2, and n3 are as described herein, including in embodiments.

In embodiments, compound (VIII-c) is

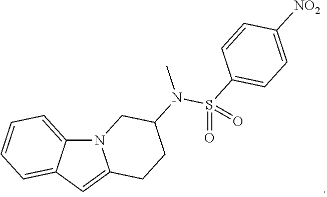 [SMILES: CN(C1CCc2cc3ccccc3n2C1)S(=O)(=O)c1ccc([N+](=O)[O-])cc1]

### VI. Embodiments

Embodiment P1. A compound, or a pharmaceutically acceptable salt thereof, having the formula:

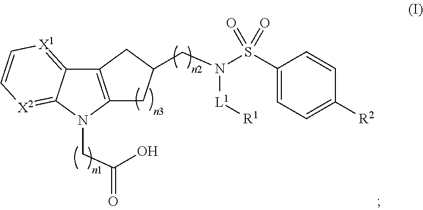

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
n1 and n2 are each independently 0, 1, 2, or 3; and
n3 is independently 0, 1, or 2.

Embodiment P2. A compound, or a pharmaceutically acceptable salt thereof, having the formula:

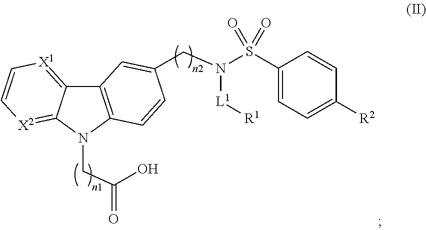

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F; and
n1 and n2 are each independently 0, 1, 2, or 3.

Embodiment P3. A compound, or a pharmaceutically acceptable salt thereof, having the formula:

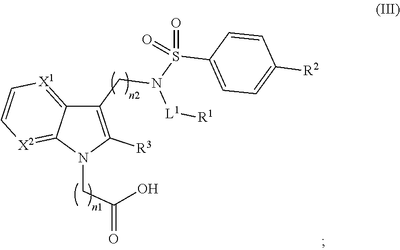

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n1 and n2 are each independently 0, 1, 2, or 3.

Embodiment P4. A compound, or a pharmaceutically acceptable salt thereof, having the formula:

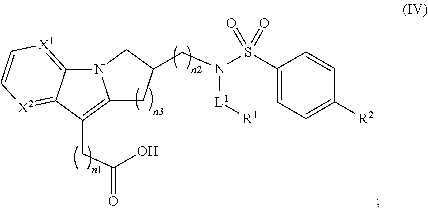

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
n1 and n2 are each independently 0, 1, 2, or 3; and
n3 is 0, 1, or 2.

Embodiment P5. The compound of one of embodiments P1 and P4, wherein n3 is 2.

Embodiment P6. The compound of embodiment P3, wherein R³ is hydrogen or unsubstituted C₁-C₄ alkyl.

Embodiment P7. The compound of embodiment P3, wherein R³ is hydrogen or unsubstituted methyl.

Embodiment P8. The compound of one of embodiments P1 to P7, wherein n1 is 1.

Embodiment P9. The compound of one of embodiments P1 to P7, wherein n1 is 2.

Embodiment P10. The compound of one of embodiments P1 to P9, wherein n2 is 0.

Embodiment P11. The compound of one of embodiments P1 to P9, wherein n2 is 2.

Embodiment P12. The compound of one of embodiments P1 to P11, wherein X¹ is CH.

Embodiment P13. The compound of one of embodiments P1 to P11, wherein X¹ is N.

Embodiment P14. The compound of one of embodiments P1 to P13, wherein X² is CH.

Embodiment P15. The compound of one of embodiments P1 to P13, wherein X² is N.

Embodiment P16. The compound of one of embodiments P1 to P15, wherein L¹ is a bond or unsubstituted C₁-C₄ alkylene.

Embodiment P17. The compound of one of embodiments P1 to P15, wherein L¹ is a bond.

Embodiment P18. The compound of one of embodiments P1 to P17, wherein R¹ is hydrogen, substituted or unsubstituted alkyl, or substituted or unsubstituted aryl.

Embodiment P19. The compound of one of embodiments P1 to P17, wherein R¹ is hydrogen, substituted or unsubstituted C₁-C₄ alkyl, or substituted or unsubstituted phenyl.

Embodiment P20. A compound, or a pharmaceutically acceptable salt thereof, having the formula:

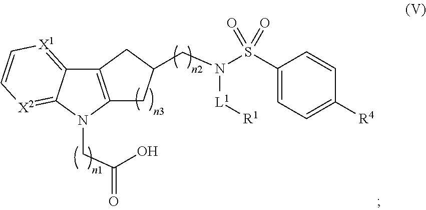

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group;
n1 and n2 are each independently 0, 1, 2, or 3; and
n3 is 0, 1, or 2.

Embodiment P21. A compound, or a pharmaceutically acceptable salt thereof, having the formula:

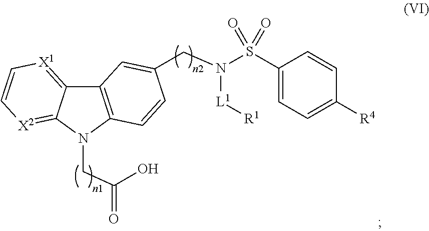

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group; and
n1 and n2 are each independently 0, 1, 2, or 3.

Embodiment P22. A compound, or a pharmaceutically acceptable salt thereof, having the formula:

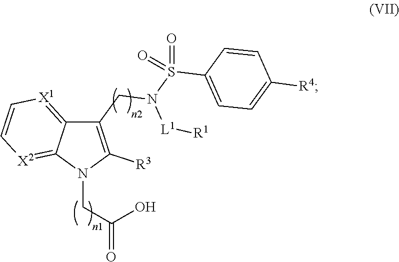

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group; and
n1 and n2 are each independently 0, 1, 2, or 3.

Embodiment P23. A compound, or a pharmaceutically acceptable salt thereof, having the formula:

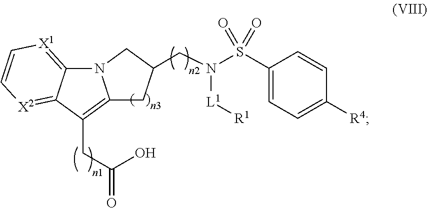

wherein
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group;
n1 and n2 are each independently 0, 1, 2, or 3; and
n3 is 0, 1, or 2.

Embodiment P24. The compound of one of embodiments P20 to P23, wherein R⁴ is —NO₂, —Cl, —Br, —I,

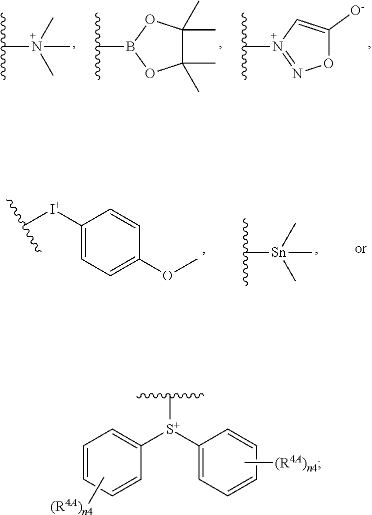

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

Embodiment P25. The compound of one of embodiments P20 to P23, wherein R⁴ is

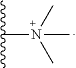

Embodiment P26. The compound of one of embodiments P20 and P23, wherein n3 is 2.

Embodiment P27. The compound of embodiment P22, wherein R³ is hydrogen or unsubstituted C₁-C₄ alkyl.

Embodiment P28. The compound of embodiment P22, wherein R³ is hydrogen or unsubstituted methyl.

Embodiment P29. The compound of one of embodiments P20 to P28, wherein n1 is 1.

Embodiment P30. The compound of one of embodiments P20 to P28, wherein n1 is 2.

Embodiment P31. The compound of one of embodiments P20 to P30, wherein n2 is 0.

Embodiment P32. The compound of one of embodiments P20 to P30, wherein n2 is 2.

Embodiment P33. The compound of one of embodiments P20 to P32, wherein X¹ is CH.

Embodiment P34. The compound of one of embodiments P20 to P32, wherein X¹ is N.

Embodiment P35. The compound of one of embodiments P20 to P34, wherein X² is CH.

Embodiment P36. The compound of one of embodiments P20 to P34, wherein X² is N.

Embodiment P37. The compound of one of embodiments P20 to P36, wherein L¹ is a bond or unsubstituted C₁-C₄ alkylene.

Embodiment P38. The compound of one of embodiments P20 to P36, wherein L¹ is a bond.

Embodiment P39. The compound of one of embodiments P20 to P38, wherein R¹ is hydrogen, substituted or unsubstituted alkyl, or substituted or unsubstituted aryl.

Embodiment P40. The compound of one of embodiments P20 to P38, wherein R¹ is hydrogen, substituted or unsubstituted C₁-C₄ alkyl, or substituted or unsubstituted phenyl.

Embodiment P41. A pharmaceutical composition comprising a compound of one of embodiments P1 to P19, or a pharmaceutically acceptable salt thereof, and a pharmaceutically acceptable excipient.

Embodiment P42. A method of detecting the level of G-protein-coupled receptor 44 in a subject, the method comprising administering to the subject an effective amount of a compound of one of embodiments P1 to P19, or a pharmaceutically acceptable salt thereof.

Embodiment P43. The method of embodiment P42, further comprising detecting a signal emitted by ¹⁸F in said compound.

Embodiment P44. The method of embodiment P43, further comprising generating an image representative of the location and/or amount of said compound based on said signal.

Embodiment P45. The method of embodiment P44, further comprising determining the distribution and/or extent of a disease in said subject.

Embodiment P46. The method of embodiment P45, wherein the disease is a metabolic disorder.

Embodiment P47. The method of embodiment P46, wherein the metabolic disorder is type 1 diabetes.

Embodiment P48. The method of embodiment P46, wherein the metabolic disorder is type 2 diabetes.

Embodiment P49. A method of detecting the level of G-protein-coupled receptor 44 in a cell, tissue, or organ, the method comprising contacting the cell, tissue, or organ with a compound of one of embodiments P1 to P19, or a pharmaceutically acceptable salt thereof.

Embodiment P50. The method of embodiment P49, further comprising detecting a signal emitted by ¹⁸F in said compound.

Embodiment P51. The method of embodiment P50, further comprising generating an image representative of the location and/or amount of said compound based on said signal.

Embodiment P52. The method of embodiment P51, further comprising determining the distribution and/or extent of a disease in said cell, tissue, or organ.

Embodiment P53. The method of embodiment P52, wherein the disease is a metabolic disorder.

Embodiment P54. The method of embodiment P53, wherein the metabolic disorder is type 1 diabetes.

Embodiment P55. The method of embodiment P53, wherein the metabolic disorder is type 2 diabetes.

Embodiment P56. A method of detecting the level of islets in a subject, the method comprising administering to the subject an effective amount of a compound of one of embodiments P1 to P19, or a pharmaceutically acceptable salt thereof.

Embodiment P57. The method of embodiment P56, wherein the subject has undergone islet transplantation for treatment of diabetes.

Embodiment P58. A method of detecting the level of beta cells in a subject, the method comprising administering to the subject an effective amount of a compound of one of embodiments P1 to P19, or a pharmaceutically acceptable salt thereof.

Embodiment P59. The method of embodiment P58, wherein the subject has received an anti-diabetic treatment or potential anti-diabetic treatment.

Embodiment P60. The method of one of embodiments P58 to P59, wherein the level of beta cells is the level of endogenous beta cells.

Embodiment P61. The method of embodiment P58, wherein the subject has undergone beta cell transplantation for treatment of diabetes.

Embodiment P62. The method of one of embodiments P56 to P61, further comprising detecting a signal emitted by ¹⁸F in said compound.

Embodiment P63. The method of embodiment P62, further comprising generating an image representative of the location and/or amount of said compound based on said signal.

Embodiment P64. The method of embodiment P63, further comprising determining the distribution and/or extent of a disease in said subject.

Embodiment P65. The method of embodiment P64, wherein the disease is a metabolic disorder.

Embodiment P66. The method of embodiment P65, wherein the metabolic disorder is type 1 diabetes.

Embodiment P67. The method of embodiment P65, wherein the metabolic disorder is type 2 diabetes.

Embodiment P68. A method of making a compound, or a pharmaceutically acceptable salt thereof, having the formula:

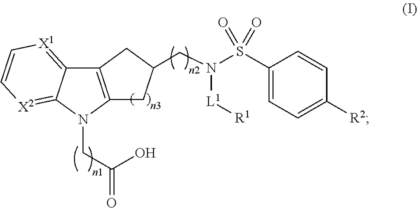

said method comprising mixing compound (V) and a ¹⁸F fluorinating agent together in a reaction vessel; wherein compound (V) has the formula:

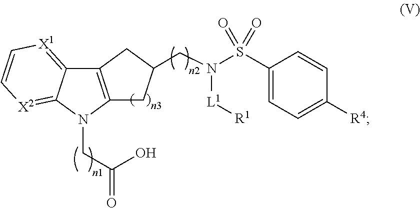

X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R⁴ is a leaving group;
n1 and n2 are each independently 0, 1, 2, or 3; and
n3 is 0, 1, or 2.

Embodiment P69. The method of embodiment P68, wherein R⁴ is —NO₂, —Cl, —Br, —I,

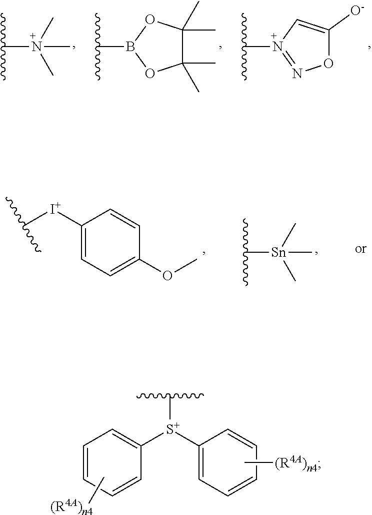

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

Embodiment P70. The method of embodiment P68, wherein R⁴ is

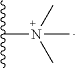

Embodiment P71. The method of one of embodiments P68 to P70, wherein the ¹⁸F fluorinating agent is K¹⁸F.

Embodiment P72. The method of one of embodiments P68 to P71, further comprising pre-treating the carboxylate group of compound (V) with a cation to make a cationic chelate.

Embodiment P73. The method of embodiment P72, wherein the cationic chelate comprises a metal cation and a chelating agent.

Embodiment P74. The method of embodiment P73, wherein the chelating agent is a cryptand.

Embodiment P75. The method of embodiment P74, wherein the cryptand is [2.2.2]-cryptand.

Embodiment P76. The method of one of embodiments P68 to P75, further comprising mixing compound (V) with a phase transfer catalyst.

Embodiment P77. The method of embodiment P76, wherein the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄.

Embodiment P78. The method of one of embodiments P70 to P77, further comprising mixing compound (V-g) and methyl triflate to make compound (V); wherein compound (V-g) has the formula:

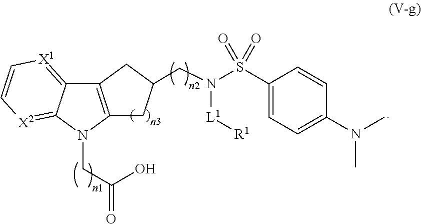

Embodiment P79. The method of embodiment P78, further comprising mixing compound (V-f), formaldehyde, and sodium cyanoborohydride to make compound (V-g); wherein compound (V-f) has the formula:

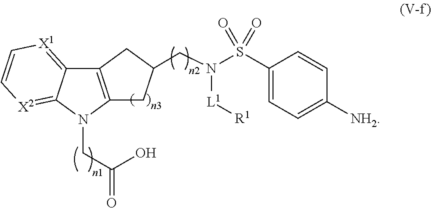

Embodiment P80. The method of embodiment P79, further comprising mixing compound (V-e), zinc, and ammonium chloride to make compound (V-f); wherein compound (V-e) has the formula:

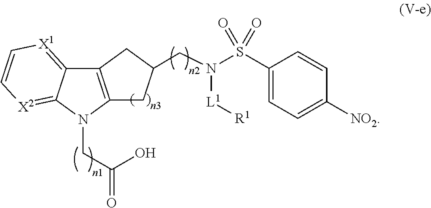

Embodiment P81. The method of embodiment P80, further comprising mixing compound (V-d) and lithium hydroxide to make compound (V-e); wherein compound (V-d) has the formula:

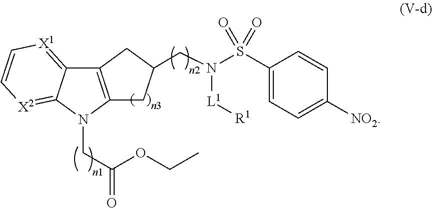

Embodiment P82. The method of embodiment P81, further comprising mixing compound (V-c), compound (c1), and sodium hydride to make compound (V-d); wherein compound (V-c) has the formula:

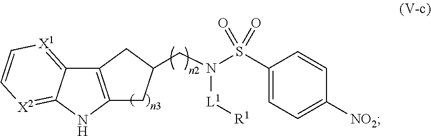

compound (c1) has the formula:

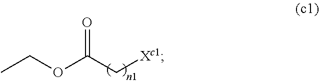

and
X^{c1} is a halogen.

Embodiment P83. A method of making a compound, or a pharmaceutically acceptable salt thereof, having the formula:

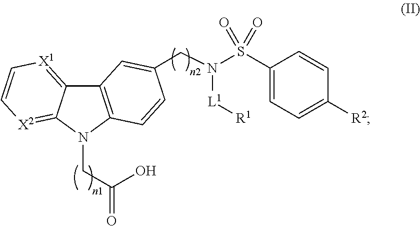

said method comprising mixing compound (VI) and a ¹⁸F fluorinating agent together in a reaction vessel; wherein

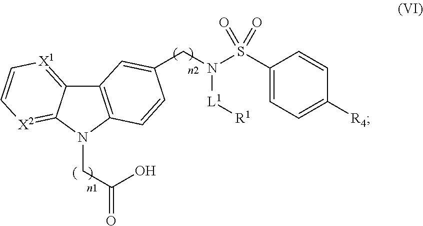

compound (VI) has the formula: 0 (VI);
X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R⁴ is a leaving group; and
n1 and n2 are each independently 0, 1, 2, or 3.

Embodiment P84. The method of embodiment P83, wherein R⁴ is
—NO₂, —Cl, —Br, —I,

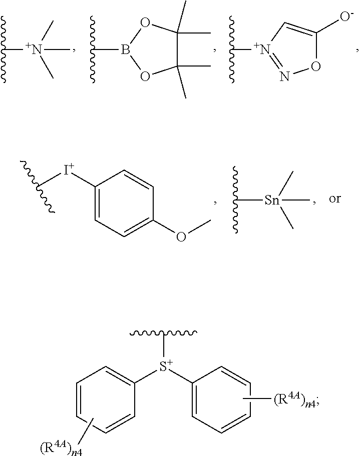

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

Embodiment P85. The method of embodiment P83, wherein R⁴ is

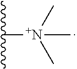 [SMILES: *[N+](C)(C)C]

Embodiment P86. The method of one of embodiments P83 to P85, wherein the ¹⁸F fluorinating agent is K¹⁸F.

Embodiment P87. The method of one of embodiments P83 to P86, further comprising pre-treating the carboxylate group of compound (VI) with a cation to make a cationic chelate.

Embodiment P88. The method of embodiment P87, wherein the cationic chelate comprises a metal cation and a chelating agent.

Embodiment P89. The method of embodiment P88, wherein the chelating agent is a cryptand.

Embodiment P90. The method of embodiment P89, wherein the cryptand is [2.2.2]-cryptand.

Embodiment P91. The method of one of embodiments P83 to P90, further comprising mixing compound (VI) with a phase transfer catalyst.

Embodiment P92. The method of embodiment P91, wherein the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄.

Embodiment P93. The method of one of embodiments P85 to P92, further comprising mixing compound (VI-g) and methyl triflate to make compound (VI); wherein compound (VI-g) has the formula:

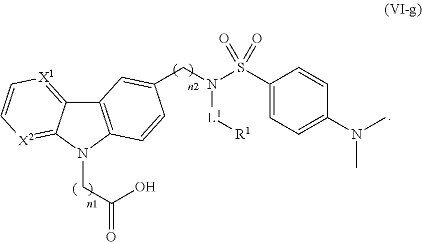

Embodiment P94. The method of embodiment P93, further comprising mixing compound (VI-f), formaldehyde, and sodium cyanoborohydride to make compound (VI-g); wherein compound (VI-f) has the formula:

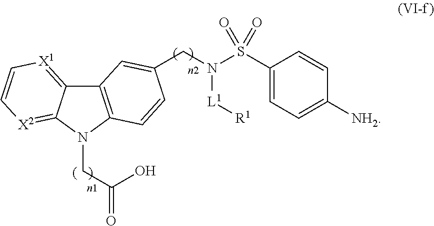

Embodiment P95. The method of embodiment P94, further comprising mixing compound (VI-e), zinc, and ammonium chloride to make compound (VI-f); wherein compound (VI-e) has the formula:

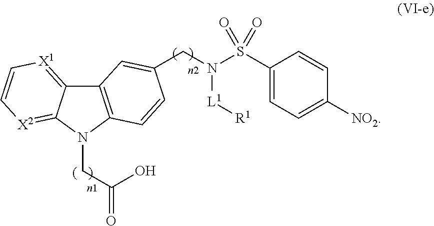

Embodiment P96. The method of embodiment P95, further comprising mixing compound (VI-d) and lithium hydroxide to make compound (VI-e); wherein compound (VI-d) has the formula:

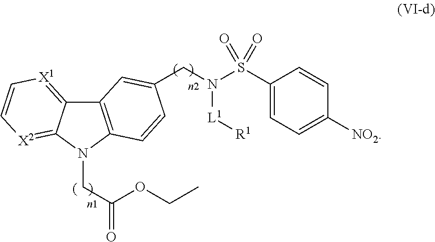

Embodiment P97. The method of embodiment P96, further comprising mixing compound (VI-c), compound (c1), and sodium hydride to make compound (VI-d); wherein compound (VI-c) has the formula:

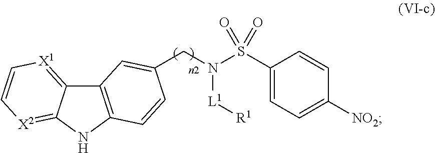

compound (c1) has the formula:

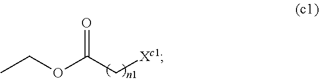

and X^{c1} is a halogen.

Embodiment P98. A method of making a compound, or a pharmaceutically acceptable salt thereof, having the formula:

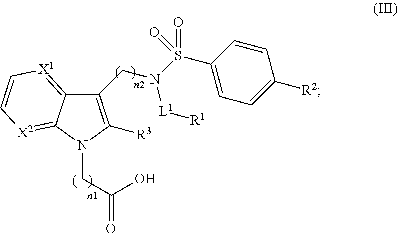

said method comprising mixing compound (VII) and a ¹⁸F fluorinating agent together in a reaction vessel; wherein
compound (VII) has the formula: 0 (VII);

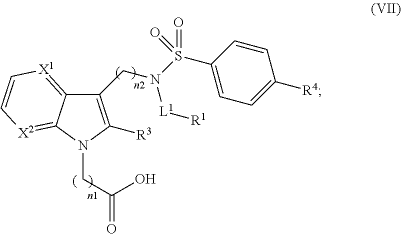

X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group; and
n1 and n2 are each independently 0, 1, 2, or 3.

Embodiment P99. The method of embodiment P98, wherein R⁴ is
—NO₂, —Cl, —Br, —I,

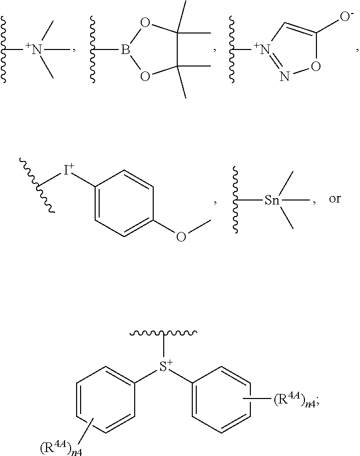

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

Embodiment P100. The method of embodiment P98, wherein R⁴ is

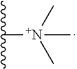 [SMILES: *[N+](C)(C)C]

Embodiment P101. The method of one of embodiments P98 to P100, wherein the ¹⁸F fluorinating agent is K¹⁸F.

Embodiment P102. The method of one of embodiments P98 to P101, further comprising pre-treating the carboxylate group of compound (VII) with a cation to make a cationic chelate.

Embodiment P103. The method of embodiment P102, wherein the cationic chelate comprises a metal cation and a chelating agent.

Embodiment P104. The method of embodiment P103, wherein the chelating agent is a cryptand.

Embodiment P105. The method of embodiment P104, wherein the cryptand is [2.2.2]-cryptand.

Embodiment P106. The method of one of embodiments P98 to P105, further comprising mixing compound (VII) with a phase transfer catalyst.

Embodiment P107. The method of embodiment P106, wherein the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄.

Embodiment P108. The method of one of embodiments P100 to P107, further comprising mixing compound (VII-g) and methyl triflate to make compound (VII); wherein compound (VII-g) has the formula: 0 (VII-g).

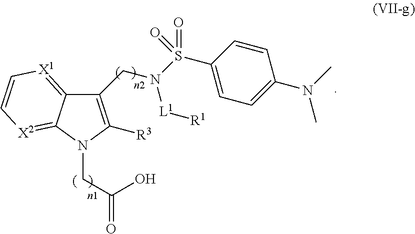

Embodiment P109. The method of embodiment P108, further comprising mixing compound (VII-f), formaldehyde, and sodium cyanoborohydride to make compound (VII-g); wherein

compound (VII-f) has the formula: 0 (VII-f).

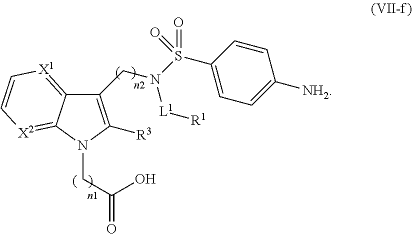

Embodiment P110. The method of embodiment P109, further comprising mixing compound (VII-e), zinc, and ammonium chloride to make compound (VII-f); wherein

compound (VII-e) has the formula: 0 (VII-e).

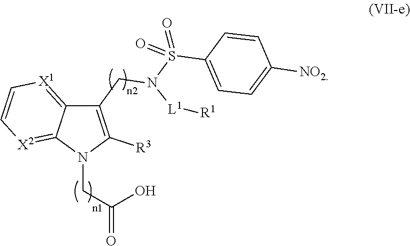

Embodiment P111. The method of embodiment P110, further comprising mixing compound (VII-d) and lithium hydroxide to make compound (VII-e); wherein

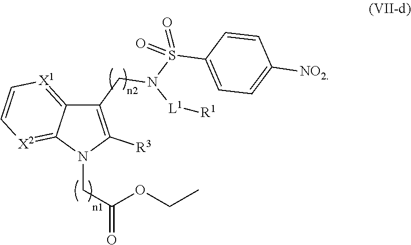

compound (VII-d) has the formula: 0 (VII-d).

Embodiment P112. The method of embodiment P111, further comprising mixing compound (VII-c), compound (c1), and sodium hydride to make compound (VII-d); wherein

compound (VII-c) has the formula:

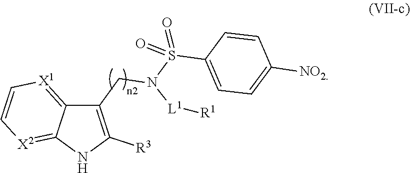

compound (c1) has the formula:

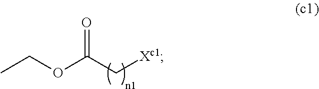

and X^{c1} is a halogen.

Embodiment P113. A method of making a compound, or a pharmaceutically acceptable salt thereof, having the formula:

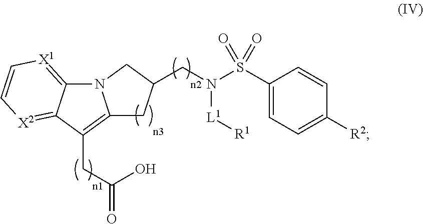

said method comprising mixing compound (VIII) and a ¹⁸F fluorinating agent together in a reaction vessel; wherein
compound (VIII) has the formula: 0 (VIII);

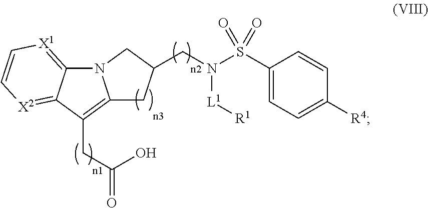

X¹ and X² are each independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R⁴ is a leaving group;
n1 and n2 are each independently 0, 1, 2, or 3; and
n3 is 0, 1, or 2.

Embodiment P114. The method of embodiment P113, wherein R⁴ is
—NO₂, —Cl, —Br, —I,

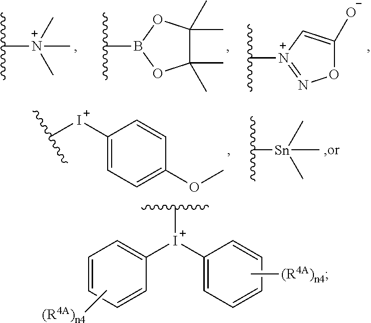

R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

Embodiment P115. The method of embodiment P113, wherein R⁴ is

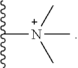 [SMILES: *[N+](C)(C)C]

Embodiment P116. The method of one of embodiments P113 to P115, wherein the ¹⁸F fluorinating agent is K¹⁸F.

Embodiment P117. The method of one of embodiments P113 to P116, further comprising pre-treating the carboxylate group of compound (VIII) with a cation to make a cationic chelate.

Embodiment P118. The method of embodiment P117, wherein the cationic chelate comprises a metal cation and a chelating agent.

Embodiment P119. The method of embodiment P118, wherein the chelating agent is a cryptand.

Embodiment P120. The method of embodiment P119, wherein the cryptand is [2.2.2]-cryptand.

Embodiment P121. The method of one of embodiments P113 to P120, further comprising mixing compound (VIII) with a phase transfer catalyst.

Embodiment P122. The method of embodiment P121, wherein the phase transfer catalyst is [K([2.2.2]-cryptand)]₂CO₃, [K([2.2.2]-cryptand)]OH, [K([2.2.2]-cryptand)]HCO₃, or [K([2.2.2]-cryptand)]₂C₂O₄.

Embodiment P123. The method of one of embodiments P115 to P122, further comprising mixing compound (VIII-g) and methyl triflate to make compound (VIII); wherein

compound (VIII-g) has the formula:

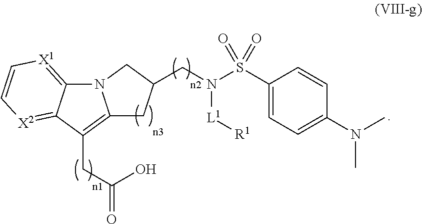

Embodiment P124. The method of embodiment P123, further comprising mixing compound (VIII-f), formaldehyde, and sodium cyanoborohydride to make compound (VIII-g); wherein

compound (VIII-f) has the formula:

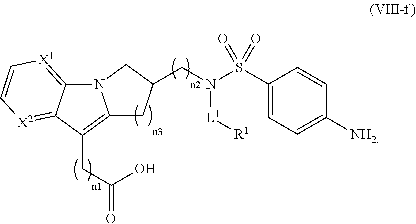

Embodiment P125. The method of embodiment P124, further comprising mixing compound (VIII-e), zinc, and ammonium chloride to make compound (VIII-f); wherein

compound (VIII-e) has the formula:

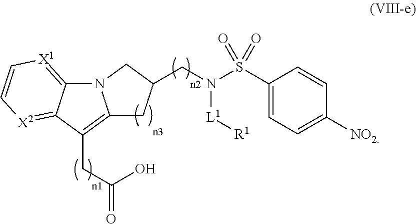

Embodiment P126. The method of embodiment P125, further comprising mixing compound (VIII-d) and lithium hydroxide to make compound (VIII-e); wherein compound (VIII-d) has the formula: 0 (VIII-d).

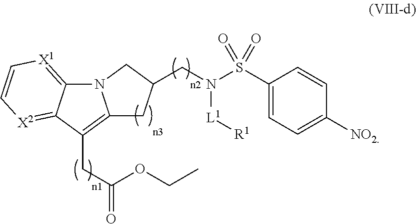

Embodiment P127. The method of embodiment P126, further comprising mixing compound (VIII-c), compound (c1I), and sodium hydride to make compound (VIII-d); wherein

compound (VIII-c) has the formula:

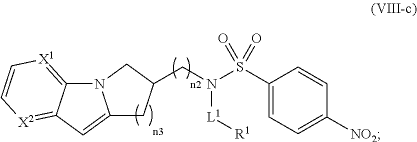

compound (c1) has the formula:

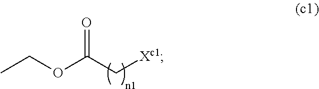

and X^{c1} is a halogen.

Embodiment P128. The method of one of embodiments P68 to P82 and P113 to P127, wherein n3 is 2.

Embodiment P129. The method of one of embodiments P98 to P112, wherein R³ is hydrogen or unsubstituted C₁-C₄ alkyl.

Embodiment P130. The method of one of embodiments P98 to P112, wherein R³ is hydrogen or unsubstituted methyl.

Embodiment P131. The method of one of embodiments P68 to P130, wherein n¹ is 1.

Embodiment P132. The method of one of embodiments P68 to P130, wherein n¹ is 2.

Embodiment P133. The method of one of embodiments P68 to P132, wherein n2 is 0.

Embodiment P134. The method of one of embodiments P68 to P132, wherein n2 is 2.

Embodiment P135. The method of one of embodiments P68 to P134, wherein X¹ is CH.

Embodiment P136. The method of one of embodiments P68 to P134, wherein X¹ is N.

Embodiment P137. The method of one of embodiments P68 to P136, wherein X² is CH.

Embodiment P138. The method of one of embodiments P68 to P136, wherein X² is N.

Embodiment P139. The method of one of embodiments P68 to P138, wherein L¹ is a bond or unsubstituted C₁-C₄ alkylene.

Embodiment P140. The method of one of embodiments P68 to P138, wherein L¹ is a bond.

Embodiment P141. The method of one of embodiments P68 to P140, wherein R¹ is hydrogen, substituted or unsubstituted alkyl, or substituted or unsubstituted aryl.

Embodiment P142. The method of one of embodiments P68 to P140, wherein R¹ is hydrogen, substituted or unsubstituted C₁-C₄ alkyl, or substituted or unsubstituted phenyl.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Examples

### Example 1: A Unique ¹⁸F-Labeled G-Protein-Coupled Receptor 44 (GPR44) Radiotracer

An estimated 285 million people, corresponding to 6.4% of the world's adult population is affected by diabetes mellitus. The development of type 1 and type 2 diabetes highly related to beta cell dysfunction, dedifferentiation and loss of beta cell mass. In vivo assessment of beta cell mass (BCM) could help to develop new treatments/therapies. Furthermore, it would promote the understanding of the progress of beta cell in the course of the disease. Some agents showed the ability to protect and promote beta cell proliferation in animals. However, confirmation of these effects in people is limited due to the challenge of directly measuring beta cell mass.

G-protein-coupled receptor 44 (GPR44), also designated as the prostaglandin D₂ receptor 2, DP₂ or CRTH2, was recently identified in the pancreas as beta cell restricted in a proteomics screening effort. Transcriptomic studies of pancreatic compartments corroborated the proteomic results, showing substantially higher GPR44 mRNA in the islets of Langerhans compared to exocrine tissue. Importantly, GPR44 was not seen in the exocrine pancreas. In human islets, GPR44 co-localized with insulin+, but not glucagon+ or somatostatin+, cells indicates that expression was limited to beta cells. In vitro and translational studies found robust expression of GPR44 in beta cells, supporting it as a target for PET probe and non-invasive imaging of beta cells.

Disclosed herein, inter alia, is the development and identification of ¹⁸F-labeled GPR44 PET probes and the use of these probes for quantitative monitoring of beta cells. A sensitive PET probe is highly desired to track low density and small number beta cell.

Positron emission tomography (PET) is of particular interest due to the high sensitivity and potential for quantification of this technology. Briefly, suitable molecules may be labeled with unstable nuclides which decay by emission of positrons, i.e., the antiparticle of the electron. When the positron encounters an electron, both particles are annihilated while generating two gamma photons, emitted in opposite directions, which can be detected by a PET scanner with reasonable temporal (seconds) and spatial (millimeters) resolution. The radiolabeled molecule can thus be traced inside a living organism, including humans, following administration. If the labeled molecule binds to a receptor with sufficient affinity and specificity, it is possible to measure the receptor density in a tissue of interest.

To improve the efficiency of developing new radiotracers, we employ GPR44-targeting antagonists or drugs that are in the clinic or being developed.

TM30089 is a selective GPR44 antagonist with excellent potency (GPR44 Ki=0.6 nM). Numerous other small molecule GPR44 antagonists have been developed. Antagonists reaching early clinical phases include, but are not limited to, ramatroban and AMG863 (both non-selective GPR44 antagonists), OC000459, AZD1981, BI671800, and MK-7246, while fevipriprant (QAW039) is currently in phase 3. In other tissues, the action of endogenous prostaglandin D2 (PGD2) on GPR44 in hair follicles has been linked to hair loss alopecia, triggering research into a potential role of GPR44 antagonists as treatment for baldness.

Several small molecule antagonists have been developed for targeting of GPR44. However, ligands optimized for imaging applications have different requirements compared to those intended for therapeutic applications. For example, oral bioavailability is imperative for a drug candidate and constitutes an important feature for candidate selection, while this is largely unimportant for an intravenously administered PET ligand. Additionally, increased lipophilicity and associated increase in off-target binding may be inconsequential for a drug candidate, while non-specific in vivo binding in the PET situation may obscure any receptor-specific binding present in the tissue of interest. See Current Diabetes Reports (2019) 19: 49.

The approach for the identification of suitable PET imaging ligands for GPR44 has included radiolabeling of some of the drugs currently or previously in clinical trials, or existing antagonists. The upside of such an approach is that the ligands are presumably thoroughly characterized (affinity, lipophilicity, protein binding, etc.) as well as tested for toxicology.

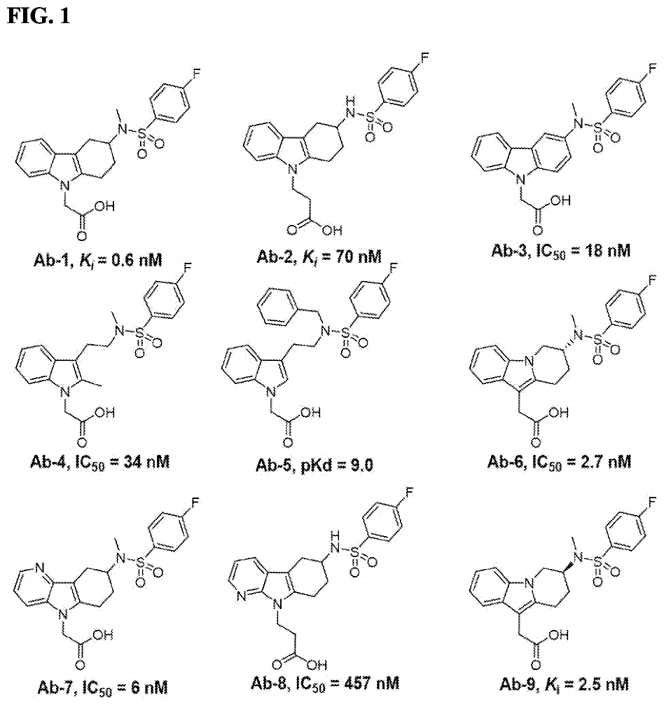

The examples shown in FIG. 1 contain a fluorine nuclide, which can be isotopically replaced by Fluorine-18 and thus generating a preferable longer radioactive half-life (108 min). Currently, there are no F-18 GPR44 PET tracers reported. These new F-18 probes will allow real-time noninvasive characterization of islet and beta cell mass. Such capabilities are key to evaluating therapies for diabetes and for improved patient selection for clinical trials. Although GPR44 is expressed preferentially in beta cell, the exact role of GPR44 in beta cell physiology is unknown. We will also explore if variation in blood glucose impacts GPR44 expression in human beta cells. GPR44-targeting PET ligands may also be useful in identifying neuroendocrine tumors.

Many of the reported ligands for GPR44 contain a free carboxylic acid group. While a ¹⁸F labeling method was reported, labeling of compounds that contain a free carboxyl group in a single step is quite challenging. However, given the short half-life of ¹⁸F and the effects on radiochemical yield, one-step labelling becomes mandatory. The GPR44 antagonists in FIG. 1 have the same labeling position of ¹⁹F and contain a free carboxyl group. The successful approach developed herein for labeling one of F-18 will permit production a library of ¹⁸F GPR44 probes.

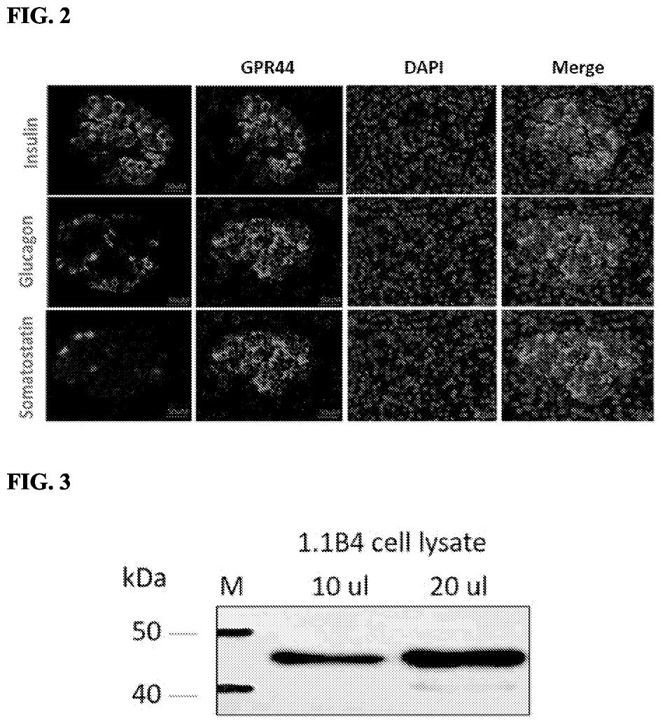

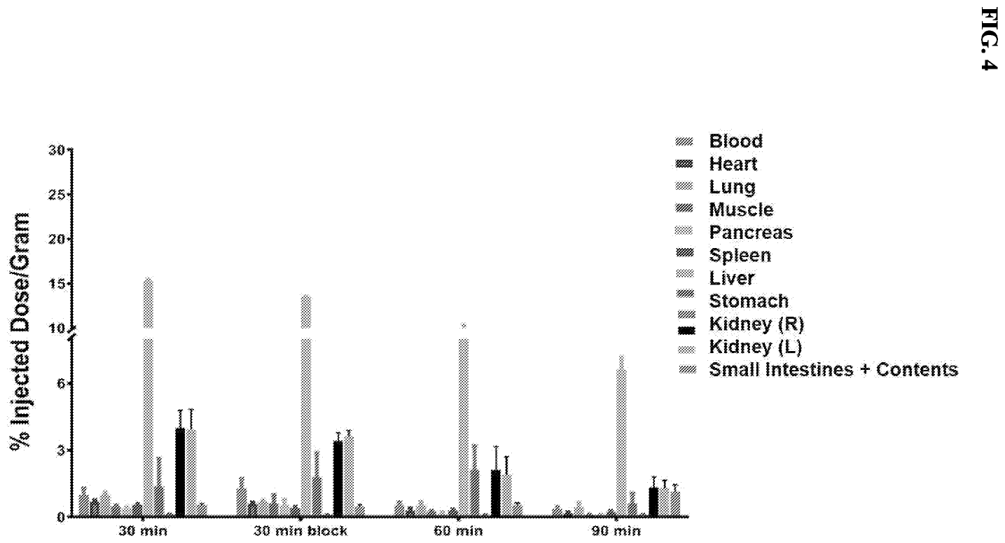

GPR44 as a reliable target for assessment of islets. Published data indicated that GPR44 was found in human islet cells. Further, GPR44 was not found in other pancreatic cells types beyond islets. Extending this, our pilot data demonstrated that islet-expressed GPR44 co-localized with cells expressing insulin (i.e., beta cells) but not cells expressing glucagon or somatostatin (FIG. 2). These findings indicate that GPR44 is specifically found on islet beta cells and support GPR44 as a rationale target to localize beta cells.

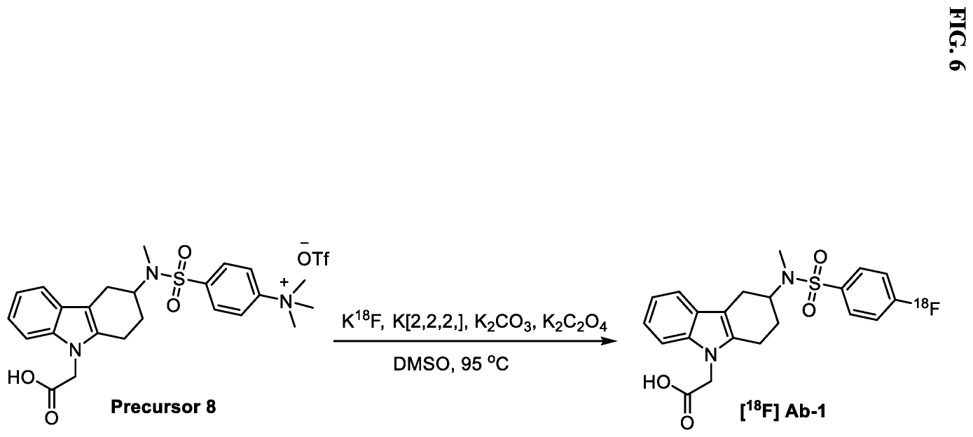

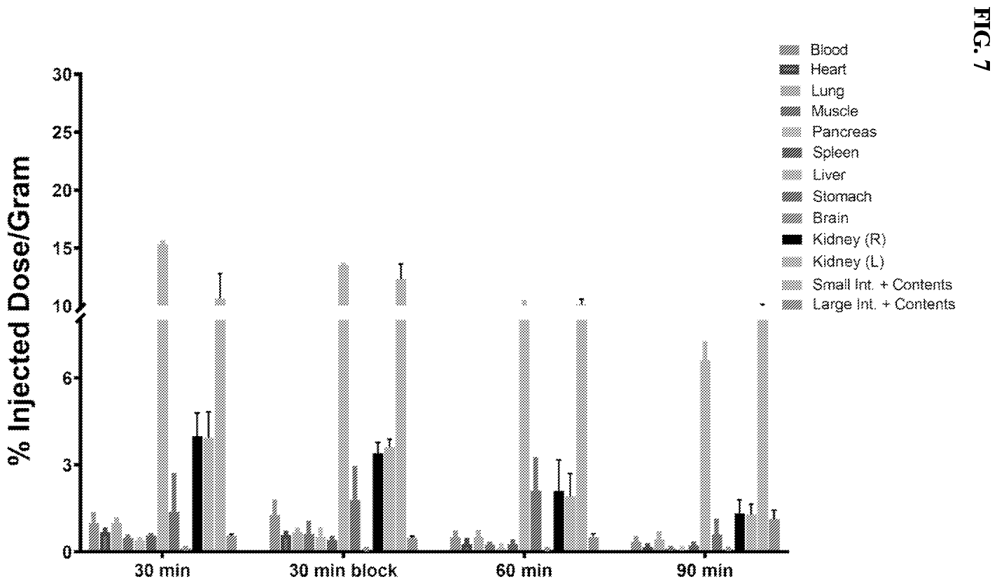

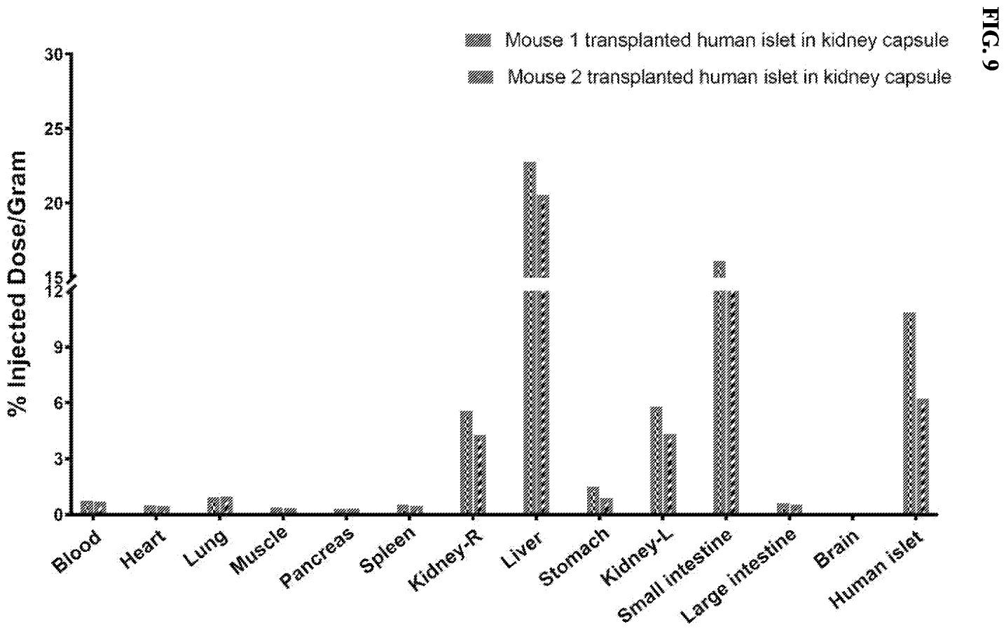

Establishment of a murine model for evaluation of GPR44 radioligands. Although GPR44 has high expression in beta cell in pigs, non-human primates and people, almost no expression is found in mice and rats. This could represent a challenge to pre-clinical development. Previously, we described a technique for transplanting human islets into the livers and kidneys of diabetic NOD/SCID or STZ-treated mice. To minimize the usage of precious human islets and provide for greater flexibility, we explored other possible alternatives. Human β-like 1.1B4 cells (or 1.2B4) produce and secrete insulin similar to fresh human islets. Human 1.1B4 cells behave in many ways exactly as normal human islets. Preliminary data indicated that human 1.1B4 cells also express GPR44 (FIG. 5 and FIG. 8), supporting the use of these cells in an initial screen of new GPR44 radioligands.

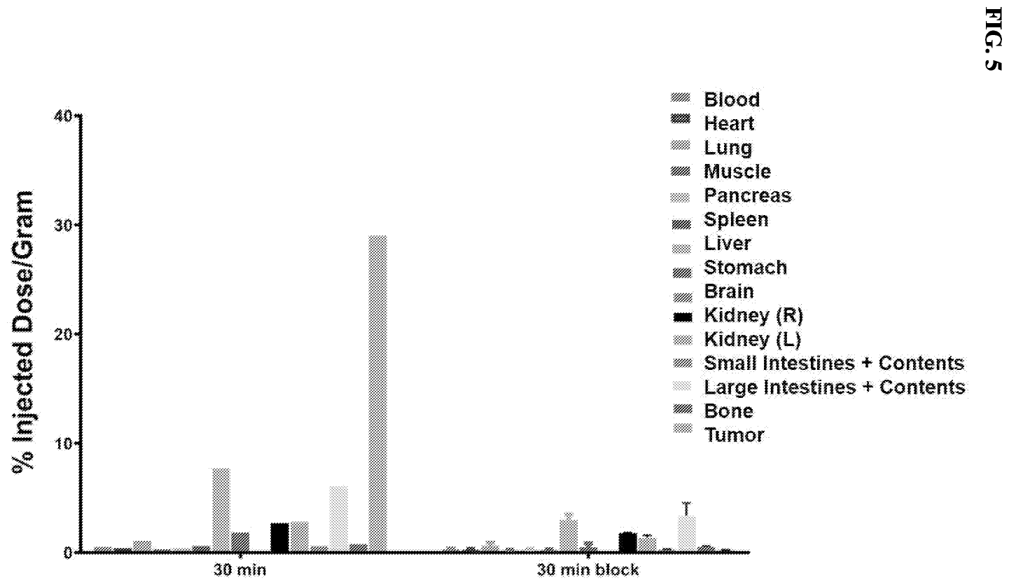

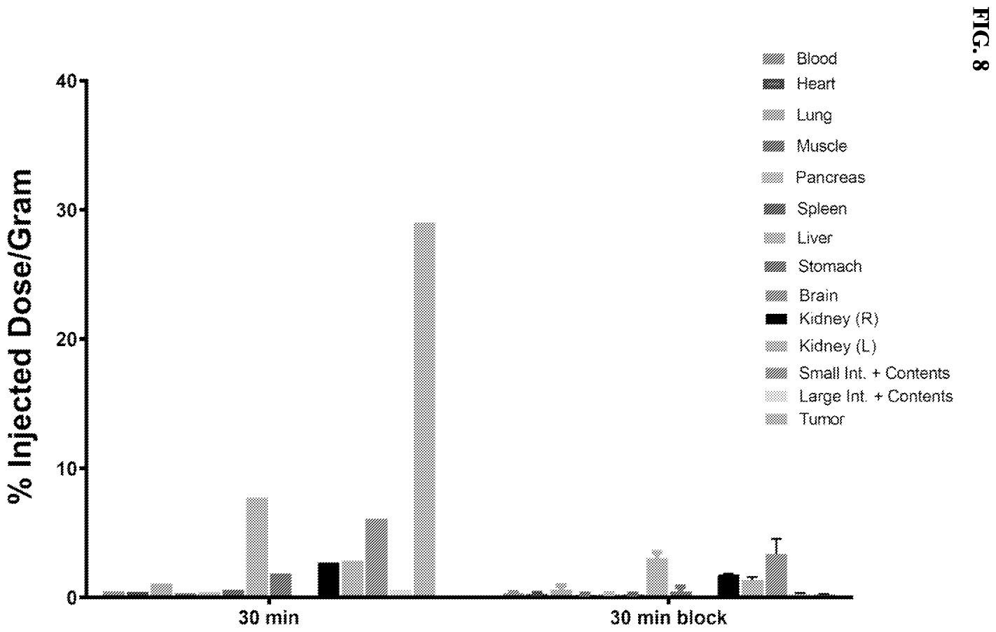

Bio-distribution analysis over time of a unique ¹⁸F GPR44 radioligand in healthy NOD/SCID mice. The biodistribution of [¹⁸F] Ab-1 was investigated in health NOD/SCID mice. The mice were administered doses of the radiotracer i.v. through the tail vein under general anesthesia. At 30, 60, and 90 minutes post-injection, mice were euthanized. The blood, lung, heart, liver, spleen, stomach, small and large intestines, muscle, kidney, pancreas and bone were collected, weighed, and radioactivity counted. Prominent uptake was in the liver and much less so in the kidneys (FIG. 4 and FIG. 7). The murine pancreas showed minimal uptake consistent with lack of GPR44 expression in this organ. We also injected cold unlabeled Ab-1, as a blocking agent, and collected the organs at 30 minutes post-injection. Organ uptake varied little from results obtained in mice given the radiotracer. As expected, it had no specific binding in murine tissues consistent with lack of GPR44 expression in the murine.

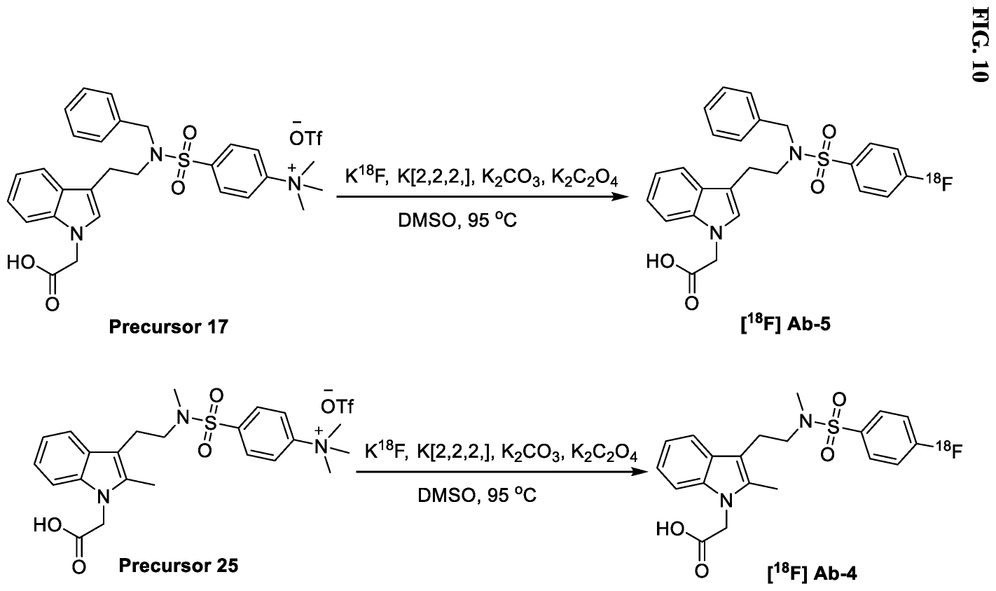

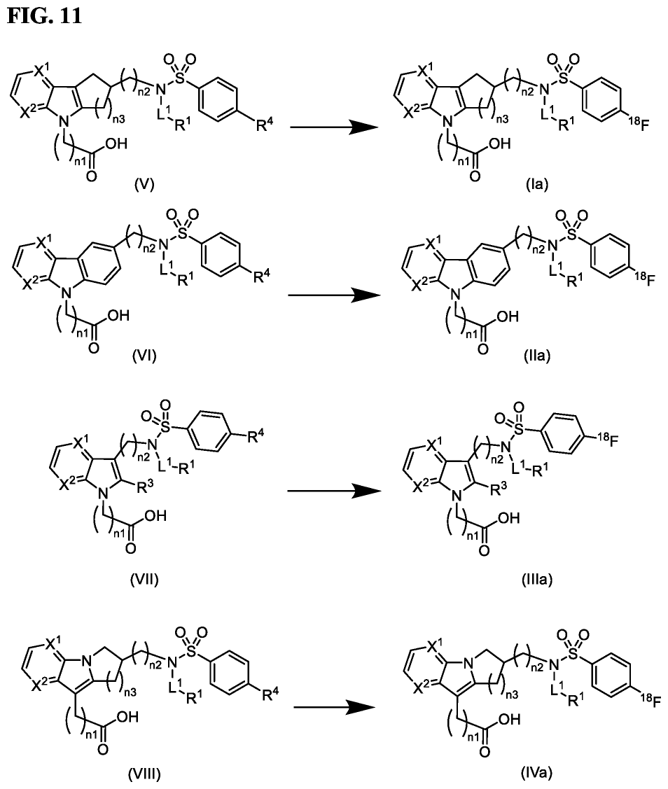

Bio-distribution analysis demonstrates specific localization of a novel ¹⁸F-labeled GPR44 radiotracer to human beta-like 1.1B4 cells/human islets implanted in mice. Preliminary studies confirmed that the human beta-like 1.1B4 cells express GPR44. However, it was not clear if the radiotracer would localize to these cells in living animals. We performed a biodistribution study in NOD/SCID mice post 3 weeks following implantation of 1.1B4 cells. The blocking agent was cold Ab-1. At 30 minutes post-injection, the mice with/without blocking agent were euthanized and organs were collected. Interestingly, 1.1B4 cells had the highest uptake of this radiotracer, and this was decreased by cold Ab-1 (FIG. 5 and FIG. 8). In other organs uptake was not different before or after administration of cold Ab-1 indicating that [¹⁸F] Ab-1 specifically binds to GPR44+1.1B4 cells. For mice with transplanted study, 500 IEQ human islets were transplanted into NOD/SCID mice kidney capsule. We also performed the biodistribution study in the mice with human islets (FIG. 9). The result indicated that human islets had significant uptake for [¹⁸F] Ab-1. Further, [¹⁸F] Ab-1 displayed high specific binding to human islets.

We showed that GPR44 is a rational target for human islet beta cells. We synthesized a GPR44-targeting PET radiotracer with high purity. Further, an initial mouse study validated that [¹⁸F] Ab-1 accumulated in GPR44+ human beta-like 1.1 B4 cells and human islets.

In addition to providing a novel technique for assessing the change in BCM in the development of metabolic disease, such imaging technology may also provide important new end-points in pharmaceutical drug development. Given the residual remaining BCM in the pancreas in subjects with long-standing T2D, as well as T1D, the expansion of endogenous beta cells may be a possible anti-diabetic treatment. For example, GLP-1 agonism has been demonstrated to expand BCM in rodent models on group level by post-mortem pancreas analysis, but it is not trivial to demonstrate a similar effect in clinical studies. A sensitive BCM imaging marker could for the first time allow such a clinical endpoint, potentially opening up novel research areas also in human individuals. Also other types of beta-cell replacement technologies could benefit from a beta-cell imaging marker, for example, the relatively established intraportal transplantation of islets to subjects with T1D (where the treatment efficiency is far from optimal) or emerging treatments such as transplantation of macro-encapsulated beta cells or stem cell transplantation.

### Example 2: Synthesis and Characterization Data

Example 1: TM30089 (Ab-1)

Synthesis of precursor of ¹⁸F-TM30089 (¹⁸F-Ab-1).

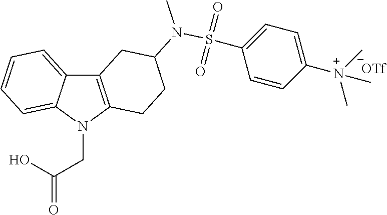

The synthetic route for precursor of ¹⁸F-Ab-1 is shown in Scheme 1. The synthetic route started with commercially available compound 1. Intermediate 2 was obtained in one step of reductive amination. Under the conditions of trimethylamine, intermediate 2 reacted with 4-nitrobenzene-1-sulfonyl chloride to give intermediate 3. Intermediate 3 successfully delivered compound 4 with Sodium hydride as base. After one step of hydrolysis, acid 5 was obtained. The nitro group on acid 5 was reduced to amino group under the condition of zinc dust in good yield. The di-methylation of the amino group could be obtained by reductive amination to yield compound 7. Finally, the trimethylammonium salt 9 was obtained under the condition of methyl triflate as methyl reagent in acetonitrile.

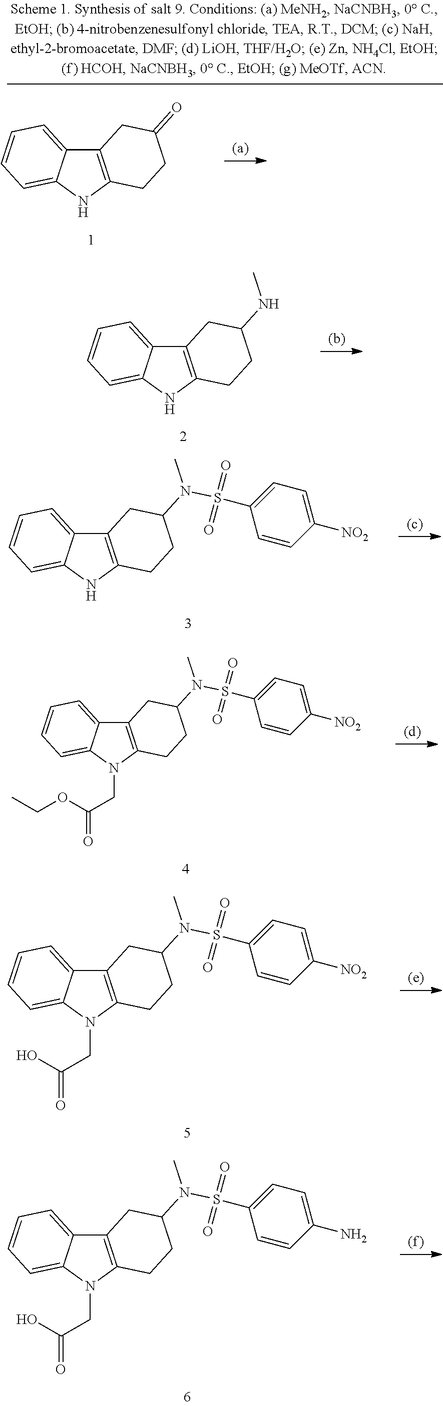 [SMILES: O=C1CCc2[nH]c3ccccc3c2C1 ; CNC1CCc2[nH]c3ccccc3c2C1 ; CN(C1CCc2[nH]c3ccccc3c2C1)S(=O)(=O)c1ccc([N+](=O)[O-])cc1 ; CCOC(=O)Cn1c2c(c3ccccc31)CC(N(C)S(=O)(=O)c1ccc([N+](=O)[O-])cc1)CC2 ; CN(C1CCc2c(c3ccccc3n2CC(=O)O)C1)S(=O)(=O)c1ccc([N+](=O)[O-])cc1 ; CN(C1CCc2c(c3ccccc3n2CC(=O)O)C1)S(=O)(=O)c1ccc(N)cc1]

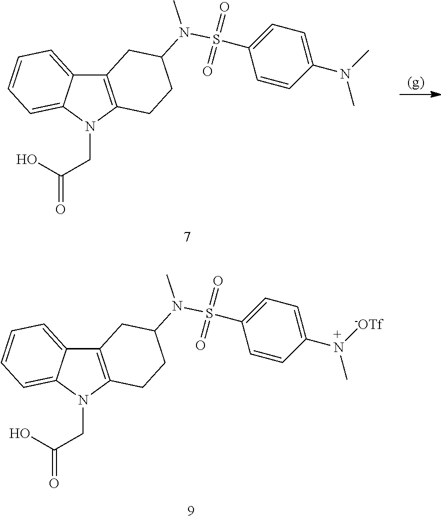

### N-methyl-2,3,4,9-tetrahydro-1H-carbazol-3-amine (2)

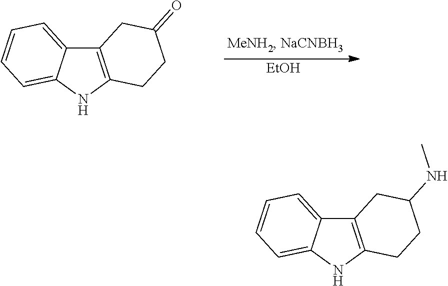

To a solution of 1,2,4,9-tetrahydro-3H-carbazol-3-one (370 mg, 1 mmol, 1 eq) in EtOH (10 mL) was added acetic acid (0.24 mL, 2 mmol, 2 eq) and MeNH₂ (30 wt. %, EtOH solution, 0.16 mL, 2 mmol, 2 eq) under nitrogen at room temperature. The resulting reaction mixture was stirred under room temperature for 30 minutes and then cooled to 0° C. The NaBH₃CN (260 mg, 2 mmol, 2 eq) was added to the reaction mixture under nitrogen at room temperature. The resulting reaction mixture allowed to warm to room temperature and stirred overnight. The reaction was quenched with water (100 mL) and ethyl acetate (100 mL). The aqueous phase was separated and extracted with ethyl acetate (3×50 mL). The organic phases were combined and washed with water (2×50 mL) and brine (2×50 mL), dried with sodium sulfate. The solvent was removed under reduced pressure. The residue was applied for next step directly.

### N-methyl-4-nitro-N-(2,3,4,9-tetrahydro-1H-carbazol-3-yl)benzenesulfonamide (3)

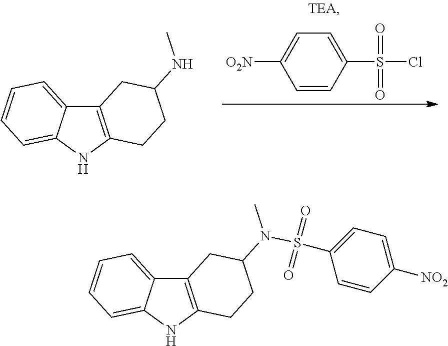 [SMILES: CNC1CCc2[nH]c3ccccc3c2C1 ; O=[N+]([O-])c1ccc(S(=O)(=O)Cl)cc1 ; CN(C1CCc2[nH]c3ccccc3c2C1)S(=O)(=O)c1ccc([N+](=O)[O-])cc1]

To a solution of residue from last step in DCM (10 mL) was added Et₃N (0.28 mL, 2 eq) at room temperature. The resulting reaction mixture was cooled to 0° C. when a solution of 4-nitrobenzenesulfonyl chloride (330 mg, 1.5 eq) in DCM (5 mL) was added dropwise. The resulting reaction mixture was stirred at 4° C. overnight. The reaction mixture was diluted with DCM (100 mL) and water (100 mL). The aqueous phase was separated and extracted with DCM (3×50 mL). The organic phases were combined and washed with water (2×50 mL) and brine (2×50 mL), dried with sodium sulfate. The solvent was removed under reduced pressure, the residue was purified by silica gel flash column, eluting with DCM to give the product as a yellow solid (160 mg, 42% in two steps). ¹H-NMR (700 Hz, DMSO-d⁶), δ 10.710 (brs, 1H), 8.42 (d, J=9.8 Hz, 2H), 8.14 (d, J=8.4 Hz, 2H), 7.27 (d, J=4.9 Hz, 1H), 7.23 (d, J=8.4 Hz, 1H), 6.99 (dd, J=7.0, 7.0 Hz, 1H), 6.91 (dd, J=7.0, 7.0 Hz 1H), 4.22 (t, J=5.6 Hz, 1H), 2.87 (s, 3H), 2.83-2.85 (m, 1H), 2.72-2.80 (m, 2H), 2.57-2.60 (m, 1H), 1.90-1.92 (m, 1H), 1.49-1.52 (m, 1H). ¹³C-NMR (176 Hz, DMSO-d⁶), 149.8, 144.9, 136.2, 133.0, 128.3, 126.9, 124.8, 120.4, 118.2, 117.1, 110.6, 106.2, 54.5, 29.9, 26.3, 24.6, 22.3.

### Ethyl 2-(3-((N-methyl-4-nitrophenyl)sulfonamido)-1,2,3,4-tetrahydro-9H-carbazol-9-yl)acetate (4)

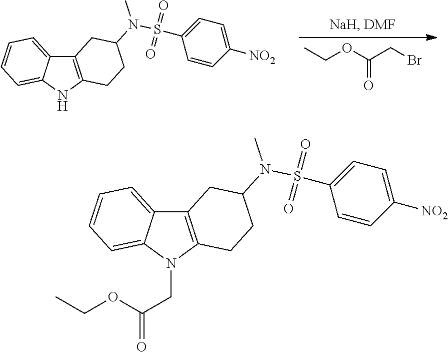

To a solution of N-methyl-4-nitro-N-(2,3,4,9-tetrahydro-1H-carbazol-3-yl)benzenesulfonamide (400 mg, 0.96 mmol, 1 eq) in DMF (4 mL) was add a suspension of NaH (62 mg, 1.1 eq) at 0° C. under nitrogen. The resulting reaction mixture was stirred for 30 min when ethyl 2-bromoacetate (0.18 mL, 1.5 eq) was added. The reaction mixture was stirred at room temperature overnight. The reaction mixture was treated with ethyl acetate (100 mL) and water (100 mL). The aqueous phase was separated and extracted with DCM (3×50 mL). The organic phases were combined and washed with water (2×50 mL) and brine (2×50 mL), dried with sodium sulfate. The solvent was removed under reduced pressure, the residue was purified by silica gel flash column, eluting with DCM to give the product as a yellow solid (410 mg). Product and starting material were inseparable. ¹H-NMR indicated that the ratio of starting material and product is 1:4. The mixture was applied for next step directly.

### 2-(3-((N-methyl-4-nitrophenyl)sulfonamido)-1,2,3,4-tetrahydro-9H-carbazol-9-yl)acetic acid (5)

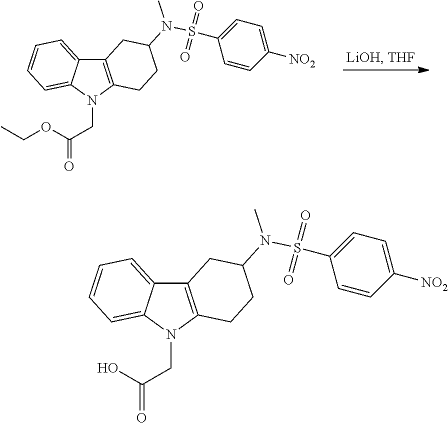 [SMILES: CCOC(=O)Cn1c2c(c3ccccc31)CC(N(C)S(=O)(=O)c1ccc([N+](=O)[O-])cc1)CC2 ; CN(C1CCc2c(c3ccccc3n2CC(=O)O)C1)S(=O)(=O)c1ccc([N+](=O)[O-])cc1]

To a solution of residue from last step (205 mg) in THF (10 mL) was added a solution of LiOH (190 mg, 1.8 eq) in water (4 mL) at room temperature under nitrogen. The resulting reaction mixture was stirred overnight. The pH of reaction mixture was adjusted to 2 with HCl (3%) and then diluted with ethyl acetate (50 mL). The reaction mixture was extracted by DCM (3×50 mL). The organic phases were combined and washed with water (2×50 mL) and brine (2×50 mL), dried with sodium sulfate. The solvent was removed under reduced pressure. The residue was purified by silica gel flash column, eluting with DCM and Methanol (10:1) to give the product as a yellow solid (106 mg, 47% in two steps). ¹H-NMR (700 MHz, DMSO-d⁶), (8.43 (d, J=8.4 Hz, 2H), 8.15 (d, J=8.4 Hz, 2H), 7.30-7.32 (m, 2H), 7.05 (dd, J=7.0, 7.0 Hz, 1H), 6.97 (dd, J=9.1, 15.4 Hz, 1H), 4.83 (s, 2H), 4.18 (t, J=5.6 Hz, 1H), 2.88 (s, 3H), 2.70-2.80 (m, 3H), 2.57-2.61 (m, 1H), 1.89-1.92 (m, 1H), 1.56-1.58 (m, 1H). ¹³C-NMR (DMSO-d⁶, 176 Hz): δ 157.2, 136.5, 131.5, 123.6, 121.0, 115.0, 113.1, 111.5, 107.4, 105.4, 104.0, 95.8, 93.2, 41.0, 30.8, 15.7, 12.8, 11.1, 7.6.

### 2-(3-((4-amino-N-methylphenyl)sulfonamido)-1,2,3,4-tetrahydro-9H-carbazol-9-yl)acetic acid (6)

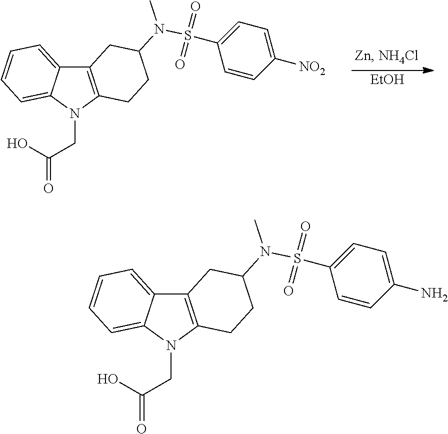 [SMILES: "EtOH" ; CN(C1CCc2c(c3ccccc3n2CC(=O)O)C1)S(=O)(=O)c1ccc([N+](=O)[O-])cc1 ; CN(C1CCc2c(c3ccccc3n2CC(=O)O)C1)S(=O)(=O)c1ccc(N)cc1]

To a solution of compound 2-(3-((N-methyl-4-nitrophenyl)sulfonamido)-1,2,3,4-tetrahydro-9H-carbazol-9-yl)acetic acid (620 mg) in EtOH (30 mL) was added NH₄C₁ (151 mg, 2 eq) and Zinc dust (896 mg, 10 eq) at room temperature. The resulting reaction mixture was stirred at 60° C. for 2 hours. The reaction mixture was filtered and washed with dichloromethane (3×100 mL) and methanol (3×100 mL). The organic solutions were combined and the solvent was removed under reduced pressure. The residue was applied for next step directly.

### 2-(3-((4-(dimethylamino)-N-methylphenyl)sulfonamido)-1,2,3,4-tetrahydro-9H-carbazol-9-yl)acetic acid (7)

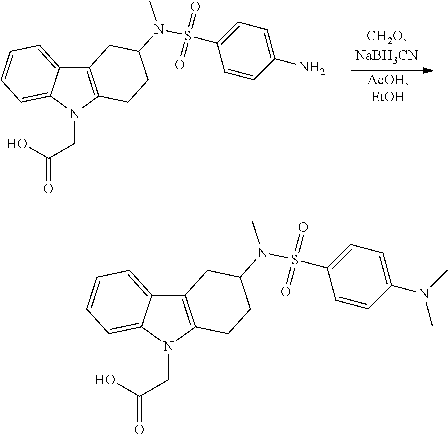

To a solution of residue from last step (150 mg, 0.36 mmol, 1 eq) and AcOH (216 mg, 10 eq) in ethanol (30 mL) was added formaldehyde solution (0.3 mL, 37% aq. 10 eq) at room temperature. The resulting reaction mixture was cooled to 0° C. and stirred at 0° C. for 1 hour, while a solution of NaBH₃CN (227 mg, 10 eq) was added in portions under Argon at 0° C. The resulting reaction mixture was stirred at 4° C. overnight. The solvent was removed under reduced pressure, the residue was purified by silica gel flash column, eluting with dichloromethane and methanol (50:1) to give the product as a white solid (68 mg, 43%). ¹H-NMR (700 MHz, DMSO-d⁶), δ 7.58 (d, J=8.4 Hz, 2H), 7.26-7.28 (m, 2H), 7.03 (t, J=7 Hz, 1H), 6.95 (t, J=7 Hz, 1H). 6.80 (d, J=8.4 Hz, 2H), 4.71 (s, 2H), 4.04 (t, J=5.6 Hz, 1H), 3.01 (s, 6H), 2.74 (s, 3H), 2.61-2.72 (m, 3H), 2.51-2.54 (m, 1H), 1.79-1.82 (m, 1H), 1.52-1.55 (m, 1H). ¹³C-NMR (176 Hz, DMSO-d⁶): δ 170.7, 152.6, 136.9, 134.5, 128.4, 126.4, 124.3, 120.4, 118.5, 117.1, 111.1, 109.1, 106.6, 54.9, 53.8, 44.7, 28.7, 25.8, 24.4, 21.1.

### 4-(N-(9-(carboxymethyl)-2,3,4,9-tetrahydro-1H-carbazol-3-yl)-N-methylsulfamoyl)-N,N,N-trimethylbenzenaminium triflate salt (8)

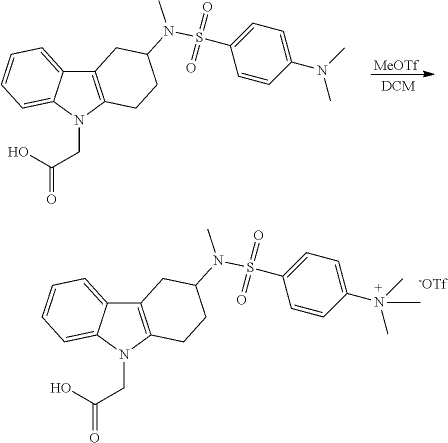

To a solution of 2-(3-((4-(dimethylamino)-N-methylphenyl)sulfonamido)-1,2,3,4-tetrahydro-9H-carbazol-9-yl)acetic acid (40 mg, 0.09 mmol, 1 eq) in DCM (2 mL) was added MeOTf (12 μL, 1.2 eq) at 0° C. The resulting reaction mixture was stirred at 0° C. for 2 hours. The solvent was removed under reduced pressure, the residue was purified by silica gel flash column to give the product as a white solid (36 mg, 67%). ¹H-NMR (700 MHz, DMSO-d⁶), δ 8.82 (d, J=9.1 Hz, 2H), 8.15 (d, J=9.1 Hz, 2H), 7.29-7.32 (m, 2H), 7.03 (dd, J=7 Hz, 7 Hz, 1H), 6.95 (d, J=7 Hz, 7 Hz, 1H), 4.82 (s, 2H), 4.20 (t, J=5.6 Hz, 1H), 3.67 (s, 9H), 2.88 (s, 3H), 2.60-2.72 (m, 3H), 2.51-2.54 (m, 1H), 1.90-1.99 (m, 1H), 1.61-1.65 (m, 1H). ¹³C-NMR (176 Hz, DMSO-d⁶): δ 170.6, 150.0, 141.1, 137.0, 134.4, 128.6, 126.4, 122.3, 120.7, 118.8, 117.2, 109.2, 106.5, 56.4, 54.2, 44.2, 28.9, 26.2, 24.4, 20.9.

### Example 2: Ab-5

Synthesis of precursor 17 4-(N-benzyl-N-(2-(1-(carboxymethyl)-1H-indol-3-yl)ethyl)sulfamoyl)-N,N,N-trimethylbenzenaminium triflate salt of ¹⁸F-Ab-5.

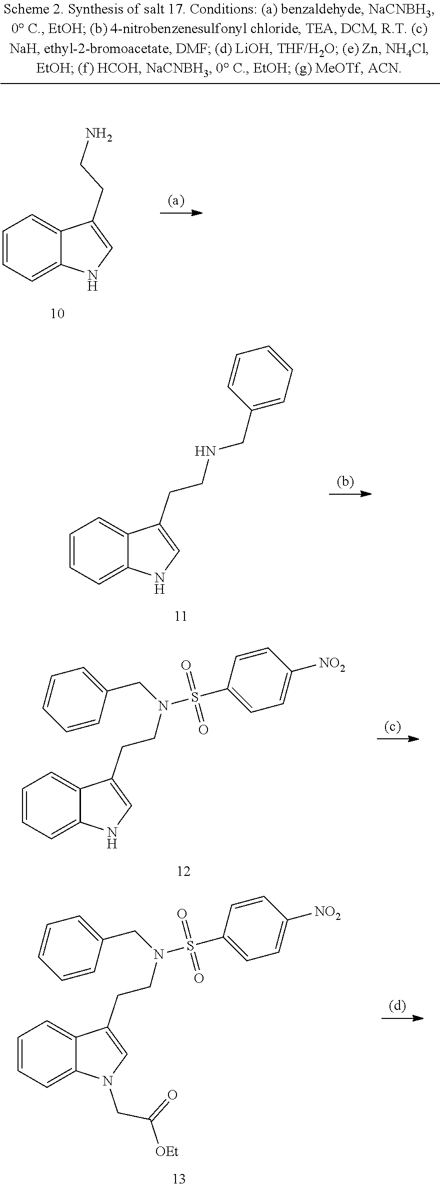 [SMILES: NCCc1c[nH]c2ccccc12 ; c1ccc(CNCCc2c[nH]c3ccccc23)cc1 ; O=[N+]([O-])c1ccc(S(=O)(=O)N(CCc2c[nH]c3ccccc23)Cc2ccccc2)cc1 ; CCOC(=O)Cn1cc(CCN(Cc2ccccc2)S(=O)(=O)c2ccc([N+](=O)[O-])cc2)c2ccccc21]

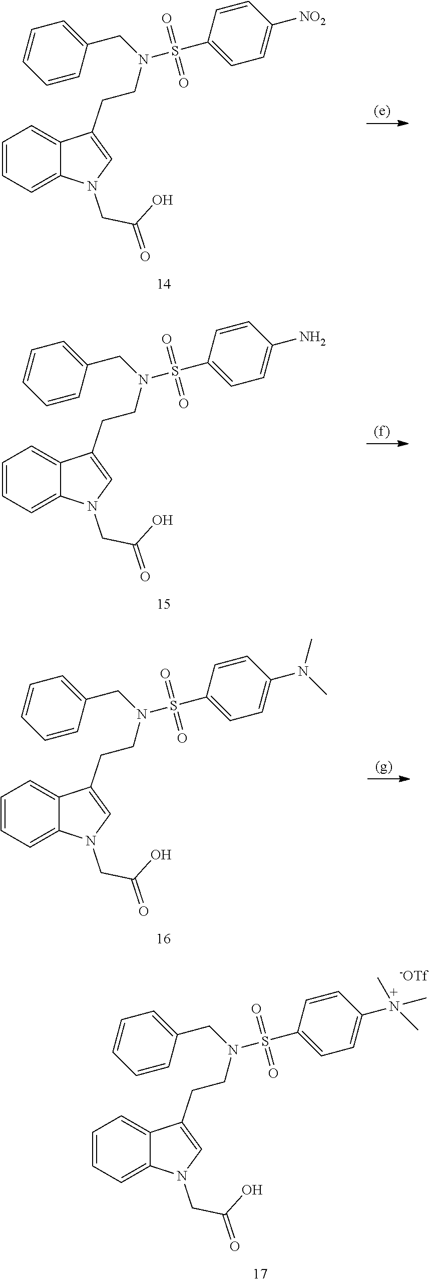

### N-benzyl-2-(1H-indol-3-yl)ethan-1-amine (11)

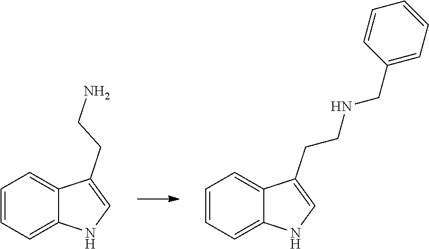 [SMILES: NCCc1c[nH]c2ccccc12 ; c1ccc(CNCCc2c[nH]c3ccccc23)cc1]

To a solution of 2-(1H-indol-3-yl)ethan-1-amine (3.2 g, 20 mmol, 1 eq) in MeOH (10 mL) was added acetic acid (2.4 mL, 40 mmol, 2 eq) nitrogen at room temperature. The NaBH₃CN (260 mg, 2 mmol, 2 eq) was added in one portion, followed by added the solution of benzaldehyde (2.12 g, 20 mmol, 1 eq) in methanol (100 mL) dropwise. The resulting reaction mixture was stirred under room temperature overnight. The reaction was quenched with water (100 mL) and ethyl acetate (100 mL). The aqueous phase was separated and extracted with ethyl acetate (3×50 mL). The organic phases were combined and washed with water (2×50 mL) and brine (2×50 mL), dried with sodium sulfate. The solvent was removed under reduced pressure. The residue was applied for next step directly.

### N-(2-(1H-indol-3-yl)ethyl)-N-benzyl-4-nitrobenzenesulfonamide (12)

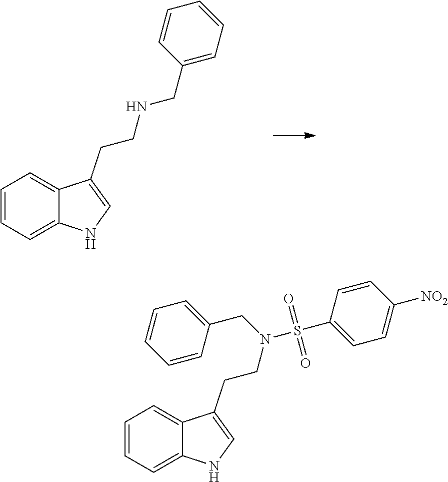 [SMILES: c1ccc(CNCCc2c[nH]c3ccccc23)cc1 ; O=[N+]([O-])c1ccc(S(=O)(=O)N(CCc2c[nH]c3ccccc23)Cc2ccccc2)cc1]

To a solution of residue from last step in DCM (200 mL) was added Et₃N (5.4 mL, 2 eq) at room temperature. The resulting reaction mixture was cooled to 0° C. when a solution of 4-nitrobenzenesulfonyl chloride (4.4 g, 1.05 eq) in DCM (25 mL) was added dropwise under nitrogen. The resulting reaction mixture was stirred at 4° C. overnight. The reaction mixture was diluted with DCM (100 mL) and water (100 mL). The aqueous phase was separated and extracted with DCM (3×50 mL). The organic phases were combined and washed with water (2×50 mL) and brine (2×50 mL), dried with sodium sulfate. The solvent was removed under reduced pressure, the residue was purified by silica gel flash column, eluting with DCM to give the product 12 as a yellow solid (4.35 g, 50% in two steps). ¹H-NMR (700 Hz, CDCl₃), δ 10.710 (brs, 1H), 8.42 (d, J=9.8 Hz, 2H), 8.14 (d, J=8.4 Hz, 2H), 7.27 (d, J=4.9 Hz, 1H), 7.23 (d, J=8.4 Hz, 1H), 6.99 (dd, J=7.0, 7.0 Hz, 1H), 6.91 (dd, J=7.0, 7.0 Hz 1H), 4.22 (t, J=5.6 Hz, 1H), 2.87 (s, 3H), 2.83-2.85 (m, 1H), 2.72-2.80 (m, 2H), 2.57-2.60 (m, 1H), 1.90-1.92 (m, 1H), 1.49-1.52 (m, 1H). ¹³C-NMR (176 Hz, CDCl₃), 150.6, 147.0, 137.3, 136.5, 130.0, 129.6, 129.37, 129.0, 128.0, 125.0, 123.4, 123.1, 120.7, 119.5, 113.2, 112.4, 52.7, 49.2, 25.6.

### Ethyl 2-(3-(2-((N-benzyl-4-nitrophenyl)sulfonamido)ethyl)-1H-indol-1-yl)acetate (13)

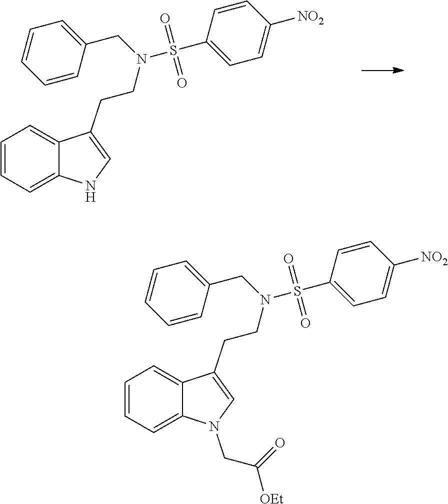 [SMILES: O=[N+]([O-])c1ccc(S(=O)(=O)N(CCc2c[nH]c3ccccc23)Cc2ccccc2)cc1 ; CCOC(=O)Cn1cc(CCN(Cc2ccccc2)S(=O)(=O)c2ccc([N+](=O)[O-])cc2)c2ccccc21]

To a solution of N-(2-(1H-indol-3-yl)ethyl)-N-benzyl-4-nitrobenzenesulfonamide (4.35 g, 10 mmol, 1 eq) in DMF (50 mL) was add a suspension of NaH (600 mg, 1.5 eq) at 0° C. under nitrogen. The resulting reaction mixture was stirred for 30 min when ethyl 2-bromoacetate (2.5 g, 1.5 eq) was added. The reaction mixture was stirred at room temperature overnight. The reaction mixture was treated with ethyl acetate (300 mL) and water (300 mL). The aqueous phase was separated and extracted with DCM (3×200 mL). The organic phases were combined and washed with water (2×200 mL) and brine (2×200 mL), dried with sodium sulfate. The solvent was removed under reduced pressure, the residue was purified by silica gel flash column, eluting with DCM to give the product 13 as a yellow solid (410 mg). The mixture was applied for next step directly.

### 2-(3-(2-((N-benzyl-4-nitrophenyl)sulfonamido)ethyl)-1H-indol-1-yl)acetic acid (14)

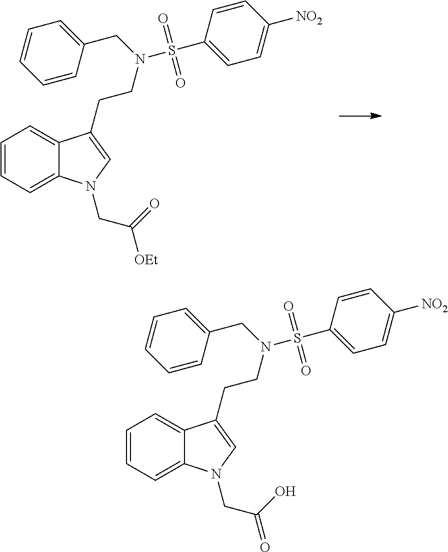 [SMILES: CCOC(=O)Cn1cc(CCN(Cc2ccccc2)S(=O)(=O)c2ccc([N+](=O)[O-])cc2)c2ccccc21 ; O=C(O)Cn1cc(CCN(Cc2ccccc2)S(=O)(=O)c2ccc([N+](=O)[O-])cc2)c2ccccc21]

To a solution of residue from last step in THF (100 mL) was added a solution of LiOH (432 mg, 1.8 eq) in water (40 mL) at room temperature under nitrogen. The resulting reaction mixture was stirred overnight. The pH of reaction mixture was adjusted to 2 with HCl (aq. 1 M) and then diluted with ethyl acetate (50 mL). The reaction mixture was extracted by DCM (3×50 mL). The organic phases were combined and washed with water (2×50 mL) and brine (2×50 mL), dried with sodium sulfate. The solvent was removed under reduced pressure. The residue was purified by silica gel flash column, eluting with DCM and methanol (10:1) to give the product 14 as a yellow solid (2.8 g, 57% in two steps). ¹H-NMR (700 Hz, DMSO-d⁶), δ 8.25 (d, J=8.4 Hz, 2H), 8.01 (d, J=8.4 Hz, 2H), 7.39-7.40 (m, 4H), 7.33 (dd, J=4.2, 4.2 Hz, 1H), 7.23 (d, J=4.8 Hz, 1H), 7.20 (dd, J=4.4 Hz 1H), 7.04-7.08 (m, 2H), 6.92 (dd, J=4.8, 4.8 Hz, 1H), 4.88 (s, 1H), 4.55 (s, 3H), 3.33-3.37 (m, 2H), 2.69-2.72 (m, 2H). ¹³C-NMR (176 Hz, DMSO-d⁶), 170.5, 149.4, 145.0, 136.5, 128.6, 128.3, 128.2, 127.8, 127.4, 127.1, 124.4, 121.2, 118.7, 118.1, 110.1, 109.6, 50.8, 47.9, 46.8, 23.9.

### 2-(3-(2-((4-amino-N-benzylphenyl)sulfonamido)ethyl)-1H-indol-1-yl)acetic acid (15)

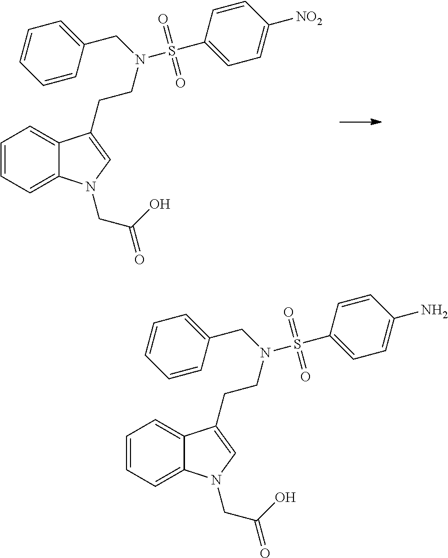 [SMILES: O=C(O)Cn1cc(CCN(Cc2ccccc2)S(=O)(=O)c2ccc([N+](=O)[O-])cc2)c2ccccc21 ; Nc1ccc(S(=O)(=O)N(CCc2cn(CC(=O)O)c3ccccc23)Cc2ccccc2)cc1]

To a solution of compound 2-(3-(2-((N-benzyl-4-nitrophenyl)sulfonamido)ethyl)-1H-indol-1-yl)acetic acid (620 mg) in EtOH (30 mL) was added NH₄Cl (151 mg, 2 eq) and Zinc dust (896 mg, 10 eq) at room temperature. The resulting reaction mixture was stirred at 60° C. for 2 hours. The reaction mixture was filtered and filtrate cake was washed with dichloromethane (3×100 mL) and methanol (3×100 mL). The organic solutions were combined and the solvent was removed under reduced pressure. The residue was applied for next step directly.

### 2-(3-(2-((N-benzyl-4-(dimethylamino)phenyl)sulfonamido)ethyl)-1H-indol-1-yl)acetic acid (16)

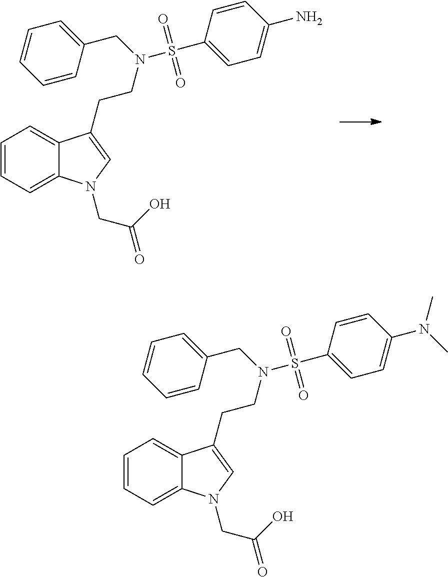 [SMILES: Nc1ccc(S(=O)(=O)N(CCc2cn(CC(=O)O)c3ccccc23)Cc2ccccc2)cc1 ; CN(C)c1ccc(S(=O)(=O)N(CCc2cn(CC(=O)O)c3ccccc23)Cc2ccccc2)cc1]

To a solution of residue from last step in ethanol (100 mL) was added AcOH (864 mg, 10 eq) and formaldehyde solution (1.2 mL, 37% aq. 10 eq) at room temperature. The resulting reaction mixture was cooled to 0° C. and stirred at 0° C. for 1 hour, while a solution of NaBH₃CN (227 mg, 10 eq) in ethanol (20 mL) was added dropwise under nitrogen at 0° C. The resulting reaction mixture was stirred at 4° C. overnight. The solvent was removed under reduced pressure, the residue was purified by silica gel flash column, eluting with dichloromethane and methanol (50:1, V/V) to give the product as a white solid (220 mg, 35% in two steps). ¹H-NMR (700 Hz, DMSO-d⁶), δ 7.63 (d, J=5.2 Hz, 2H), 7.34-7.40 (m, 4H), 7.28-7.31 (m, 1H), 7.21 (d, J=4.8 Hz, 1H), 7.15 (d, J=4.4 Hz 1H), 7.04-7.08 (m, 1H), 6.88-6.93 (m, 4H), 6.79 (d, J=5.2 Hz, 2H), 4.63 (s, 2H), 4.33 (s, 2H), 3.16-3.19 (m, 2H), 2.99 (s, 1H), 2.61-2.64 (m, 2H). ¹³C-NMR (176 Hz, DMSO-d⁶), 170.7, 152.6, 137.4, 136.5, 128.6, 128.4, 128.2, 127.5, 127.3, 127.0, 124.2, 120.8, 118.2, 117.9, 111.1, 109.8, 109.7, 51.5, 48.5, 48.4, 24.3.

### 4-(N-benzyl-N-(2-(1-(carboxymethyl)-1H-indol-3-yl)ethyl)sulfamoyl)-N,N,N-trimethylbenzenaminium triflate salt (17)

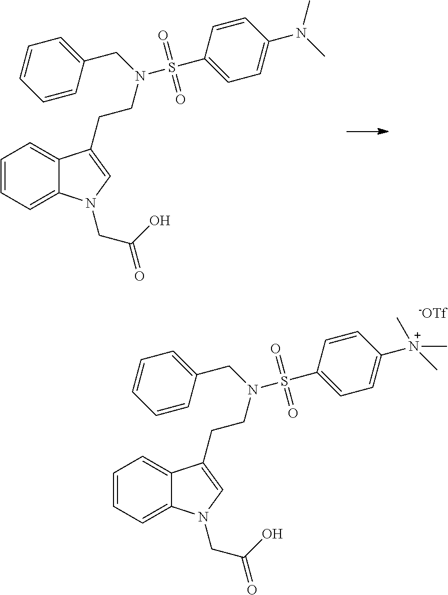 [SMILES: CN(C)c1ccc(S(=O)(=O)N(CCc2cn(CC(=O)O)c3ccccc23)Cc2ccccc2)cc1 ; C[N+](C)(C)c1ccc(S(=O)(=O)N(CCc2cn(CC(=O)O)c3ccccc23)Cc2ccccc2)cc1 ; "-OTf"]

To a solution of 2-(3-(2-((N-benzyl-4-(dimethylamino)phenyl)sulfonamido)ethyl)-1H-indol-1-yl)acetic acid (220 mg, 0.45 mmol, 1 eq) in DCM (2 mL) was added methyl triflate (60 μL, 1.2 eq) at 0° C. The resulting reaction mixture was stirred at 0° C. for 2 hours. The solvent was removed under reduced pressure, the residue was purified by silica gel flash column to give the product as a white solid (200 mg, 68%). ¹H-NMR (700 Hz, DMSO-d⁶), δ 8.17 (d, J=5.2 Hz, 2H), 8.12 (d, J=5.2 Hz, 2H), 7.34-7.40 (m, 4H), 7.31-7.34 (m, 1H), 7.29 (d, J=4.8 Hz, 1H), 7.25 (d, J=4.4 Hz 1H), 7.09 (dd, J=4.0, 4.0 Hz, 1H), 7.07, (s, 1H), 6.97 (dd, J=4.0, 4.0 Hz, 1H), 4.89 (s, 2H), 4.52 (s, 2H), 3.64 (s, 9H), 3.29-3.32 (m, 2H), 2.59-2.63 (m, 2H). ¹³C-NMR (176 Hz, DMSO-d⁶), 170.4, 149.9, 141.1, 136.6, 136.5, 128.6, 128.2, 128.8, 127.3, 127.1, 122.2, 121.3, 118.7, 118.1, 110.1, 109.8, 56.4, 51.6, 48.6, 46.9, 24.5.

### Example 3: Ab-4

Synthesis of precursor 25 4-(N-(2-(1-(carboxymethyl)-1H-indol-3-yl)ethyl)-N-methylsulfamoyl)-N,N,N-trimethylbenzenaminium triflate salt of ¹⁸F-Ab-4.

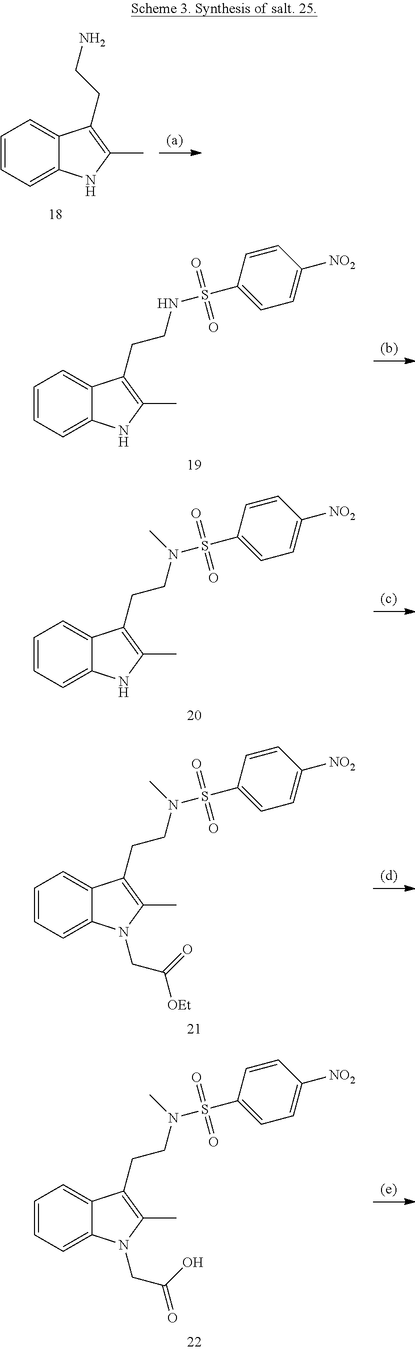 [SMILES: Cc1[nH]c2ccccc2c1CCN ; Cc1[nH]c2ccccc2c1CCNS(=O)(=O)c1ccc([N+](=O)[O-])cc1 ; Cc1[nH]c2ccccc2c1CCN(C)S(=O)(=O)c1ccc([N+](=O)[O-])cc1 ; CCOC(=O)Cn1c(C)c(CCN(C)S(=O)(=O)c2ccc([N+](=O)[O-])cc2)c2ccccc21 ; Cc1c(CCN(C)S(=O)(=O)c2ccc([N+](=O)[O-])cc2)c2ccccc2n1CC(=O)O]

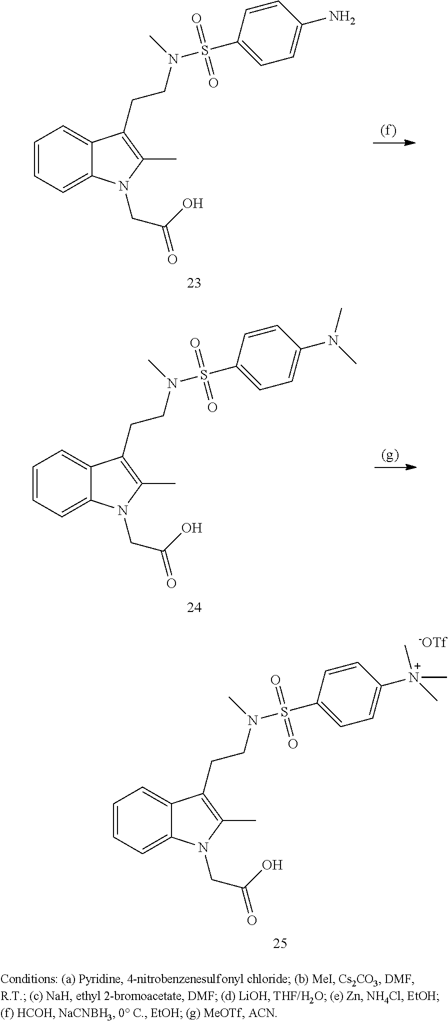 [SMILES: Cc1c(CCN(C)S(=O)(=O)c2ccc(N)cc2)c2ccccc2n1CC(=O)O ; Cc1c(CCN(C)S(=O)(=O)c2ccc(N(C)C)cc2)c2ccccc2n1CC(=O)O ; Cc1c(CCN(C)S(=O)(=O)c2ccc([N+](C)(C)C)cc2)c2ccccc2n1CC(=O)O ; "-OTf"]

### N-(2-(2-methyl-1H-indol-3-yl)ethyl)-4-nitrobenzenesulfonamide (19)

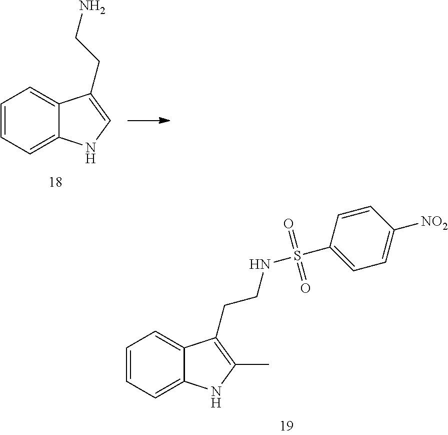 [SMILES: NCCc1c[nH]c2ccccc12 ; Cc1[nH]c2ccccc2c1CCNS(=O)(=O)c1ccc([N+](=O)[O-])cc1]

To a solution of 2-(2-methyl-1H-indol-3-yl)ethan-1-amine (500 mg, 2.87 mmol, 1 eq) and Pyridine (453 mg, 2 eq) in Acetonitrile (20 mL) was added 4-nitrobenzenesulfonyl chloride (700 mg, 1.1 eq) at room temperature under Argon. The resulting reaction mixture was stirred at room temperature overnight. The reaction mixture was treated with ethyl acetate (300 mL) and water (300 mL). The aqueous phase was separated and extracted with DCM (3×200 mL). The organic phases were combined and washed with water (2×200 mL) and brine (2×200 mL), dried with sodium sulfate. The solvent was removed under reduced pressure, the residue was purified by silica gel flash column, eluting with DCM to give the product 19 as a yellow solid.

### 2-(2-methyl-3-(2-((N-methyl-4-nitrophenyl)sulfonamido)ethyl)-1H-indol-1-yl)acetic acid (20)

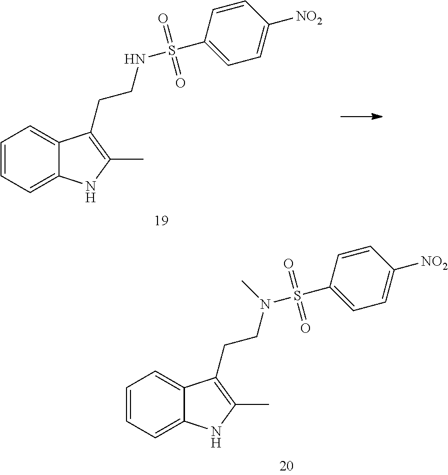 [SMILES: Cc1[nH]c2ccccc2c1CCNS(=O)(=O)c1ccc([N+](=O)[O-])cc1 ; Cc1[nH]c2ccccc2c1CCN(C)S(=O)(=O)c1ccc([N+](=O)[O-])cc1]

To a solution of N-(2-(2-methyl-1H-indol-3-yl)ethyl)-4-nitrobenzenesulfonamide (800 mg, 2.4 mmol, 1 eq) and Cs₂CO₃ (936 mg, 1.2 eq) in DMF (5 mL) was added methyl iodide (358 mg, 1.1 eq) at room temperature under Argon. The resulting reaction mixture was stirred at room temperature overnight. The reaction mixture was treated with ethyl acetate (300 mL) and water (300 mL). The aqueous phase was separated and extracted with DCM (3×200 mL). The organic phases were combined and washed with water (2×200 mL) and brine (2×200 mL), dried with sodium sulfate. The solvent was removed under reduced pressure, the residue was purified by silica gel flash column, eluting with DCM to give the product 20 as a yellow solid (300 mg). The mixture was applied for next step directly.

### Ethyl 2-(2-methyl-3-(2-((N-methyl-4-nitrophenyl)sulfonamido)ethyl)-1H-indol-1-yl)acetate (21)

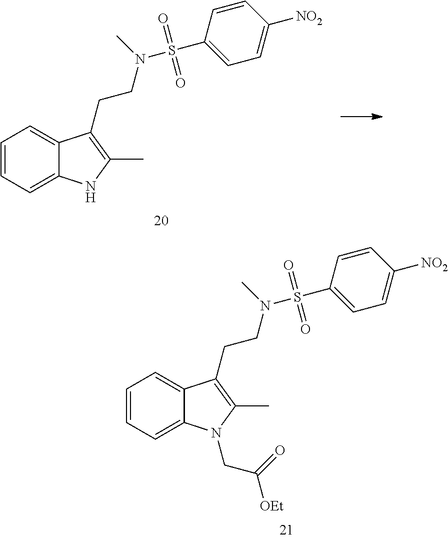 [SMILES: Cc1[nH]c2ccccc2c1CCN(C)S(=O)(=O)c1ccc([N+](=O)[O-])cc1 ; CCOC(=O)Cn1c(C)c(CCN(C)S(=O)(=O)c2ccc([N+](=O)[O-])cc2)c2ccccc21]

To a solution of N-(2-(1H-indol-3-yl)ethyl)-N-benzyl-4-nitrobenzenesulfonamide (300 mg, 2.3 mmol, 1 eq) in DMF (50 mL) was add a suspension of NaH (60 mg, 1.5 eq) at 0° C. under nitrogen. The resulting reaction mixture was stirred for 30 min when ethyl 2-bromoacetate (578 mg, 1.5 eq) was added. The reaction mixture was stirred at room temperature overnight. The reaction mixture was treated with ethyl acetate (300 mL) and water (300 mL). The aqueous phase was separated and extracted with DCM (3×200 mL). The organic phases were combined and washed with water (2×200 mL) and brine (2×200 mL), dried with sodium sulfate. The solvent was removed under reduced pressure, the residue was purified by silica gel flash column, eluting with DCM to give the product 21 as a yellow solid. The mixture was applied for next step directly.

### 2-(2-methyl-3-(2-((N-methyl-4-nitrophenyl)sulfonamido)ethyl)-1H-indol-1-yl)acetic acid (22)

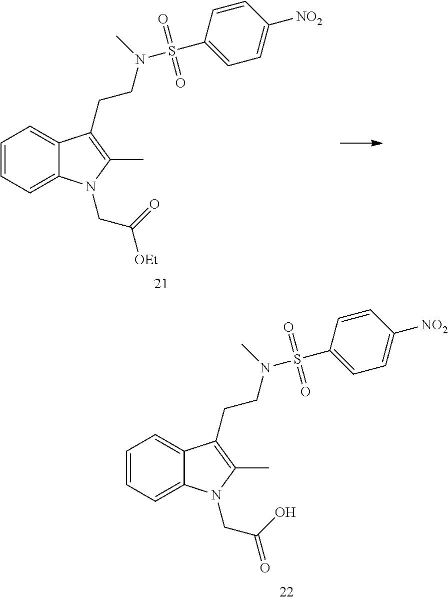 [SMILES: CCOC(=O)Cn1c(C)c(CCN(C)S(=O)(=O)c2ccc([N+](=O)[O-])cc2)c2ccccc21 ; Cc1c(CCN(C)S(=O)(=O)c2ccc([N+](=O)[O-])cc2)c2ccccc2n1CC(=O)O]

To a solution of residue from last step in THF (100 mL) was added a solution of LiOH (80 mg, 1.8 eq) in water (40 mL) at room temperature under nitrogen. The resulting reaction mixture was stirred overnight. The pH of reaction mixture was adjusted to 2 with HCl (aq. 1 M) and then diluted with ethyl acetate (50 mL). The reaction mixture was extracted by DCM (3×50 mL). The organic phases were combined and washed with water (2×50 mL) and brine (2×50 mL), dried with sodium sulfate. The solvent was removed under reduced pressure. The residue was purified by silica gel flash column, eluting with DCM and methanol (10:1) to give the product 22 as a yellow solid.

### 2-(3-(2-((4-amino-N-methylphenyl)sulfonamido)ethyl)-2-methyl-1H-indol-1-yl)acetic acid (23)

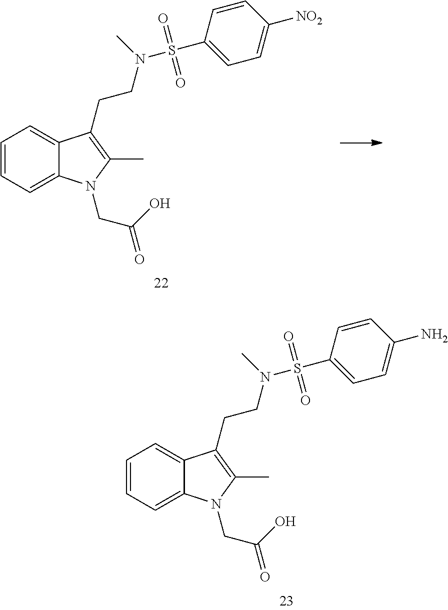 [SMILES: Cc1c(CCN(C)S(=O)(=O)c2ccc([N+](=O)[O-])cc2)c2ccccc2n1CC(=O)O ; Cc1c(CCN(C)S(=O)(=O)c2ccc(N)cc2)c2ccccc2n1CC(=O)O]

To a solution of compound 2-(3-(2-((N-benzyl-4-nitrophenyl)sulfonamido)ethyl)-1H-indol-1-yl)acetic acid (620 mg) in EtOH (30 mL) was added NH₄Cl (151 mg, 2 eq) and Zinc dust (896 mg, 10 eq) at room temperature. The resulting reaction mixture was stirred at 60° C. for 2 hours. The reaction mixture was filtered and filtrate cake was washed with dichloromethane (3×100 mL) and methanol (3×100 mL). The organic solutions were combined and the solvent was removed under reduced pressure. The residue was applied for next step directly.

### 2-(3-(2-((4-(dimethylamino)-N-methylphenyl)sulfonamido)ethyl)-2-methyl-1H-indol-1-yl)acetic acid (24)

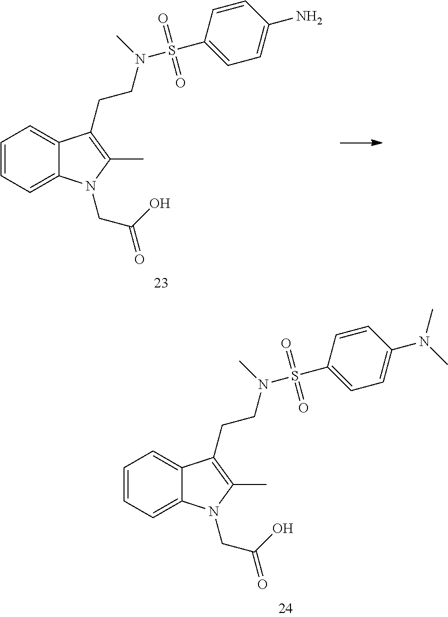 [SMILES: Cc1c(CCN(C)S(=O)(=O)c2ccc(N)cc2)c2ccccc2n1CC(=O)O ; Cc1c(CCN(C)S(=O)(=O)c2ccc(N(C)C)cc2)c2ccccc2n1CC(=O)O]

To a solution of residue from last step in ethanol (100 mL) was added AcOH (864 mg, 10 eq) and formaldehyde solution (1.2 mL, 37% aq. 10 eq) at room temperature. The resulting reaction mixture was cooled to 0° C. and stirred at 0° C. for 1 hour, while a solution of NaBH₃CN (227 mg, 10 eq) in ethanol (20 mL) was added dropwise under nitrogen at 0° C. The resulting reaction mixture was stirred at 4° C. overnight. The solvent was removed under reduced pressure, the residue was purified by silica gel flash column, eluting with dichloromethane and methanol (50:1, V/V) to give the product as a white sold.

### 4-(N-(2-(1-(carboxymethyl)-1H-indol-3-yl)ethyl)-N-methylsulfamoyl)-N,N,N-trimethylbenzenaminium triflate salt (25)

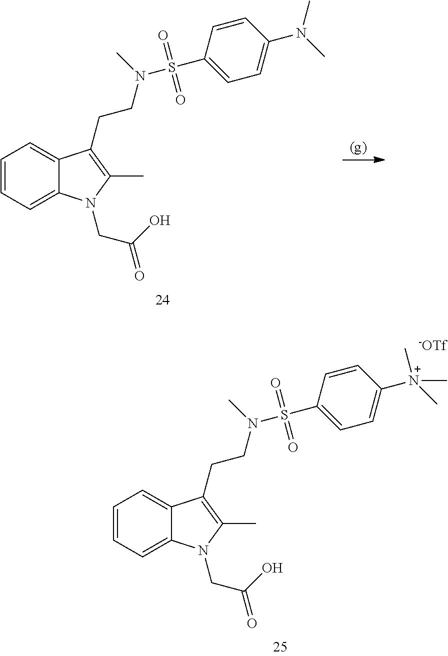 [SMILES: Cc1c(CCN(C)S(=O)(=O)c2ccc(N(C)C)cc2)c2ccccc2n1CC(=O)O ; Cc1c(CCN(C)S(=O)(=O)c2ccc([N+](C)(C)C)cc2)c2ccccc2n1CC(=O)O ; "-OTf"]

To a solution of 2-(3-(2-((N-benzyl-4-(dimethylamino)phenyl)sulfonamido)ethyl)-1H-indol-1-yl)acetic acid (100 mg, 1 eq) in DCM (2 mL) was added methyl triflate (60 μL, 1.2 eq) at 0° C. The resulting reaction mixture was stirred at 0° C. for 2 hours. The solvent was removed under reduced pressure, the residue was purified by silica gel flash column to give the product as a white solid.

General labeling experiments:

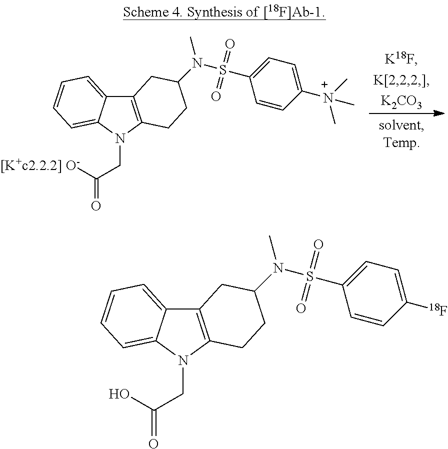

Preparation of [K+c2.2.2]₂CO₃

[K⁺c2.2.2]₂CO₃ is prepared by addition of 1.2 equivalents of Kryptofix 2.2.2 to one equivalent of K₂CO₃ in water/MeCN (50 vol %/50 vol %). The solvent is evaporated under reduced pressure and freeze dried.

Preparation of [K+c2.2.2]₂C₂O₄

[K⁺c2.2.2]₂CO₃ is prepared by addition of 1.2 equivalents of Kryptofix 2.2.2 to one equivalent of K₂C₂O₄ in water/MeCN (50 vol %/50 vol %). The solvent is evaporated under reduced pressure and freeze dried.

Pretreatment of precursor 4-(N-(9-(carboxymethyl)-2,3,4,9-tetrahydro-1H-carbazol-3-yl)-N-methylsulfamoyl)-N,N,N-trimethylbenzenaminium:

4-(N-(9-(carboxymethyl)-2,3,4,9-tetrahydro-1H-carbazol-3-yl)-N-methylsulfamoyl)-N,N,N-trimethylbenzenaminium (2 mg), K₂CO₃ Kryptofix 2.2.2 (5 mg) and K₂C₂O₄ Kryptofix 2.2.2 (12 mg) was dissolved in acetonitrile (1 mL). The resulting mixture was set under vacuum to remove the solvent. The residue was applied for labeling reaction without further treatment.

Aqueous [¹⁸F]fluoride ion was passed through a Sep-Pak Light Waters Accell Plus QMA Cartridge and eluted with a solution of K₂CO₃ (20 mg/mL in H₂O, 0.2 mL) and Kryptofix 2.2.2 (20 mg/mL in CH₃CN, 0.9 mL) into a reaction vessel. The solvent was evaporated under a stream of N₂ at 95° C. CH₃CN (1.0 mL) was added and the solvent was evaporated at 95° C. Again CH₃CN (1.0 mL) was added and the solvent was evaporated at 95° C. To the residue was added a solution of pretreated precursor (19 mg) in DMSO (1 mL) and the resulting reaction mixture was heated at 95° C. for 10 min. After cooling, HPLC buffer (CH₃CN, 1.0 mL and AMF, 1.0 mL, 0.1 M, pH 4.5) was added and the solution was transferred to the loading tube and HCl (aq. 0.8 mL, 1 N) was added. The mixture was transferred to a semipreparative HPLC system, eluted with CH₃CN/AMF (0.1 M, pH 4.5)=45/55, 4.5 mL/min), [¹⁸F]Ab-1 eluted at 15 min. The fraction containing [¹⁸F]Ab-1 was collected and concentrated under speed vacuum for 15 min, and the product was reformulated in saline and sterilized by filtration. The radiochemical purity was assessed by analytical HPLC and the identity was confirmed by co-elution with TM30089 in a spiked sample. Analytical column: eluent: A=water with 0.1% TFA; B=acetonitrile; 1 mL/min flow rate.

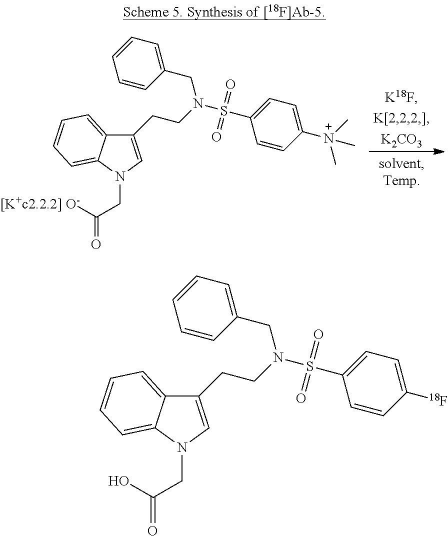

Preparation of [K+c2.2.2]₂CO₃

[K⁺c2.2.2]₂CO₃ is prepared by addition of 1.2 equivalents of Kryptofix 2.2.2 to one equivalent of K₂CO₃ in water/MeCN (50 vol %/50 vol %). The solvent is evaporated under reduced pressure and freeze dried.

Preparation of [K+c2.2.2]₂C₂O₄

[K⁺c2.2.2]₂CO₃ is prepared by addition of 1.2 equivalents of Kryptofix 2.2.2 to one equivalent of K₂C₂O₄ in water/MeCN (50 vol %/50 vol %). The solvent is evaporated under reduced pressure and freeze dried.

Pretreatment of precursor 4-(N-benzyl-N-(2-(1-(carboxymethyl)-1H-indol-3-yl)ethyl)sulfamoyl)-N,N,N-trimethylbenzenaminium:

4-(N-benzyl-N-(2-(1-(carboxymethyl)-1H-indol-3-yl)ethyl)sulfamoyl)-N,N,N-trimethylbenzenaminium (2 mg), K2CO₃ Kryptofix 2.2.2 (5 mg) and K₂C₂O₄ Kryptofix 2.2.2 (12 mg) was dissolved in acetonitrile (1 mL). The resulting mixture was set under vacuum to remove the solvent. The residue was applied for labeling reaction without further treatment.

Aqueous [¹⁸F]fluoride ion was passed through a Sep-Pak Light Waters Accell Plus QMA Cartridge and eluted with a solution of K₂CO₃ (20 mg/mL in H₂O, 0.2 mL) and Kryptofix 2.2.2 (20 mg/mL in CH₃CN, 0.9 mL) into a reaction vessel. The solvent was evaporated under a stream of N₂ at 95° C. CH₃CN (1.0 mL) was added and the solvent was evaporated at 95° C. Again CH₃CN (1.0 mL) was added and the solvent was evaporated at 95° C. To the residue was added a solution of pretreated precursor (19 mg) in DMSO (1 mL) and the resulting reaction mixture was heated at 95° C. for 10 min. After cooling, HPLC buffer (CH₃CN, 1.0 mL and AMF, 1.0 mL, 0.1 M, pH 4.5) was added and the solution was transferred to the loading tube and HCl (aq. 0.8 mL, 1 N) was added. The mixture was transferred to a semipreparative HPLC system, eluted with CH₃CN/AMF (0.1 M, pH 4.5)=45/55, 4.5 mL/min), [¹⁸F]Ab-5 eluted at 21 min. The fraction containing [¹⁸F]Ab-5 was collected and concentrated under speed vacuum for 15 min, and the product was reformulated in saline and sterilized by filtration. The radiochemical purity was assessed by analytical HPLC and the identity was confirmed by co-elution with Ab-5 in a spiked sample. Analytical column: eluent: A=water with 0.1% TFA; B=acetonitrile; 1 mL/min flow rate.

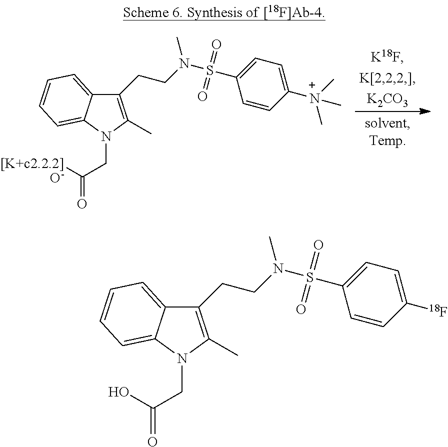

Preparation of [K+c2.2.2]₂CO₃

[K⁺c2.2.2]₂CO₃ is prepared by addition of 1.2 equivalents of Kryptofix 2.2.2 to one equivalent of K₂CO₃ in water/MeCN (50 vol %/50 vol %). The solvent is evaporated under reduced pressure and freeze dried.

Preparation of [K+c2.2.2]₂C₂O₄

[K⁺c2.2.2]₂CO₃ is prepared by addition of 1.2 equivalents of Kryptofix 2.2.2 to one equivalent of K₂C₂O₄ in water/MeCN (50 vol %/50 vol %). The solvent is evaporated under reduced pressure and freeze dried.

Pretreatment of precursor 4-(N-(2-(1-(carboxymethyl)-1H-indol-3-yl)ethyl)-N-methylsulfamoyl)-N,N,N-trimethylbenzenaminium:

4-(N-(2-(1-(carboxymethyl)-1H-indol-3-yl)ethyl)-N-methylsulfamoyl)-N,N,N-trimethylbenzenaminium (2 mg), K2CO₃ Kryptofix 2.2.2 (5 mg) and K₂C₂O₄ Kryptofix 2.2.2 (12 mg) was dissolved in acetonitrile (1 mL). The resulting mixture was set under vacuum to remove the solvent. The residue was applied for labeling reaction without further treatment.

Aqueous [¹⁸F]fluoride ion was passed through a Sep-Pak Light Waters Accell Plus QMA Cartridge and eluted with a solution of K₂CO₃ (20 mg/mL in H₂O, 0.2 mL) and Kryptofix 2.2.2 (20 mg/mL in CH₃CN, 0.9 mL) into a reaction vessel. The solvent was evaporated under a stream of N₂ at 95° C. CH₃CN (1.0 mL) was added and the solvent was evaporated at 95° C. Again CH₃CN (1.0 mL) was added and the solvent was evaporated at 95° C. To the residue was added a solution of pretreated precursor (19 mg) in DMSO (1 mL) and the resulting reaction mixture was heated at 95° C. for 10 min. After cooling, HPLC buffer (CH₃CN, 1.0 mL and AMF, 1.0 mL, 0.1 M, pH 4.5) was added and the solution was transferred to the loading tube and HCl (aq. 0.8 mL, 1 N) was added. The mixture was transferred to a semipreparative HPLC system, eluted with CH₃CN/AMF (0.1 M, pH 4.5)=45/55, 4.5 mL/min), [¹⁸F]Ab-4 eluted at 14 min. The fraction containing [¹⁸F]Ab-4 was collected and concentrated under speed vacuum for 15 min, and the product was reformulated in saline and sterilized by filtration. The radiochemical purity was assessed by analytical HPLC and the identity was confirmed by co-elution with Ab-4 in a spiked sample. Analytical column: eluent: A=water with 0.1% TFA; B=acetonitrile; 1 mL/min flow rate.

1. A compound, or a pharmaceutically acceptable salt thereof, having the formula: 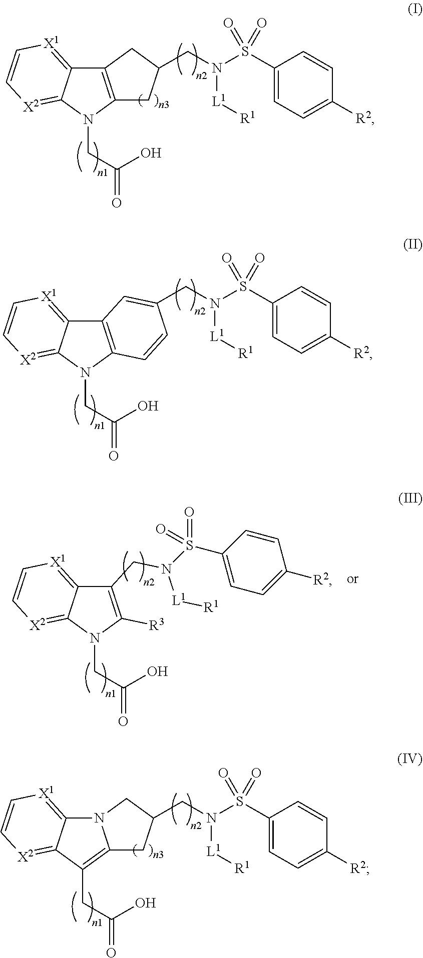
wherein
X¹ and X² are independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n1 and n2 are independently 0, 1, 2, or 3; and
n3 is 0, 1, or 2.

2.-4. (canceled)

5. The compound of claim 1, wherein n3 is 2.

6. The compound of claim 1, wherein R³ is hydrogen or unsubstituted C₁-C₄ alkyl.

7. (canceled)

8. The compound of claim 1, wherein n1 is 1.

9. (canceled)

10. The compound of claim 1, wherein n2 is 0.

11.-15. (canceled)

16. The compound of claim 1, wherein L¹ is a bond or unsubstituted C₁-C₄ alkylene.

17. (canceled)

18. (canceled)

19. The compound of claim 1, wherein R¹ is hydrogen, substituted or unsubstituted C₁-C₄ alkyl, or substituted or unsubstituted phenyl.

20. A compound, or a pharmaceutically acceptable salt thereof, having the formula: 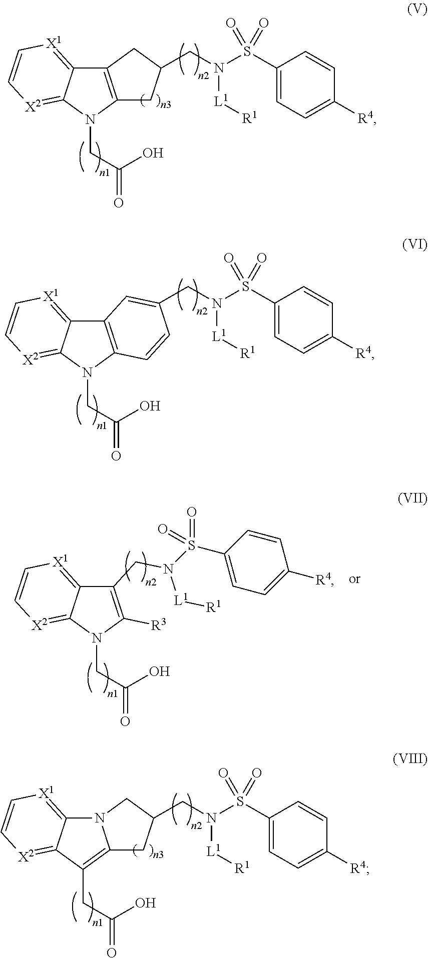
wherein
X¹ and X² are independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is a leaving group;
n1 and n2 are independently 0, 1, 2, or 3; and
n3 is 0, 1, or 2.

21.-23. (canceled)

24. The compound of claim 20, wherein R⁴ is
—NO₂, —Cl, —Br, —I, 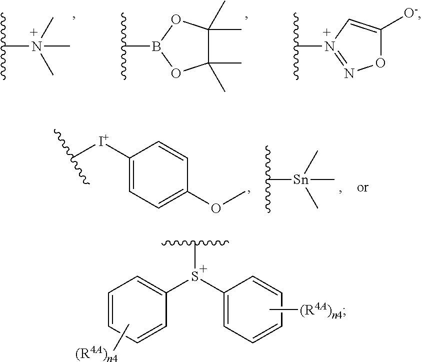
R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

25. (canceled)

26. The compound of claim 20, wherein n3 is 2.

27. The compound of claim 20, wherein R³ is hydrogen or unsubstituted C₁-C₄ alkyl.

28.-36. (canceled)

37. The compound of claim 20, wherein L¹ is a bond or unsubstituted C₁-C₄ alkylene.

38. (canceled)

39. (canceled)

40. The compound of claim 20, wherein R¹ is hydrogen, substituted or unsubstituted C₁-C₄ alkyl, or substituted or unsubstituted phenyl.

41. A pharmaceutical composition comprising a compound of claim 1, or a pharmaceutically acceptable salt thereof, and a pharmaceutically acceptable excipient.

42. A method of detecting the level of G-protein-coupled receptor 44 in a subject, the method comprising administering to the subject an effective amount of a compound of claim 1, or a pharmaceutically acceptable salt thereof.

43.-48. (canceled)

49. A method of detecting the level of G-protein-coupled receptor 44 in a cell, tissue, or organ, the method comprising contacting the cell, tissue, or organ with a compound of claim 1, or a pharmaceutically acceptable salt thereof.

50.-55. (canceled)

56. A method of detecting the level of islets in a subject, the method comprising administering to the subject an effective amount of a compound of claim 1, or a pharmaceutically acceptable salt thereof.

57. (canceled)

58. A method of detecting the level of beta cells in a subject, the method comprising administering to the subject an effective amount of a compound of claim 1, or a pharmaceutically acceptable salt thereof.

59.-67. (canceled)

68. A method of making a compound, or a pharmaceutically acceptable salt thereof, having the formula: 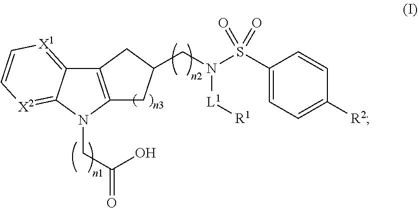
said method comprising mixing compound (V) and a ¹⁸F fluorinating agent together in a reaction vessel; wherein
compound (V) has the formula: 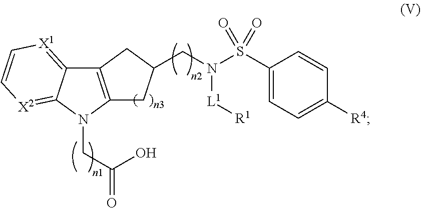
X¹ and X² are independently CH or N;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is —¹⁸F;
R⁴ is a leaving group;
n1 and n2 are independently 0, 1, 2, or 3; and
n3 is 0, 1, or 2.

69. The method of claim 68, wherein R⁴ is —NO₂, —Cl, —Br, —I, 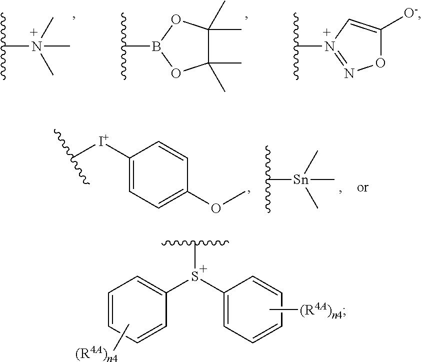
R^{4A} is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
n4 is independently an integer from 0 to 5.

70.-142. (canceled)
